(19) United States
(12) Patent Application Publication
(10) Pub. No.: US 2019/0070124 A1
(43) Pub. Date: Mar. 7, 2019

(54) COMPOSITIONS OF CB2 RECEPTOR SELECTIVE AGONISTS FOR TREATMENT OF MENTAL DISORDERS

(71) Applicant: Sharon Anavi-Goffer, Oranit (IL)

(72) Inventor: Sharon Anavi-Goffer, Oranit (IL)

(21) Appl. No.: 16/081,101

(22) PCT Filed: Mar. 3, 2017
(86) PCT No.: PCT/IB2017/000256

§ 371 Date: Aug. 30, 2018

Related U.S. Application Data
(60) Provisional application No. 62/303,508, filed on Mar. 4, 2016.
(60) Provisional application No. 62/303,494, filed on Mar. 4, 2016.

(51) Int. Cl. A61K 31/015; A61K 45/06; A61K 31/09; A61K 31/336; A61P 25/24; A61P 25/18; A61P 25/22; A61P 25/08

(52) U.S. Cl. CPC A61K 31/015; A61K 45/06; A61K 31/09; A61P 25/08; A61P 25/24; A61P 25/18; A61P 25/22; A61K 31/336

(57) ABSTRACT

Disclosed are stable compositions comprising at least one CB2 receptor selective or highly selective agonist and optionally at least one antipsychotic for use in the treatment of mental disorders, methods of preparing such compositions and methods of treating mental disorders using same. Disclosed are also compositions comprising beta caryophyllene (BCP) or HU-308 for use in the treatment of mental disorders one of which is schizophrenia, methods of making such compositions and methods of treating mental disorders one of which is schizophrenia.

### CROSS REFERENCE TO RELATED APPLICATIONS

This application claims priority to U.S. Provisional Patent Application Ser. No. 62/303,494, filed on Mar. 4, 2016, and to U.S. Provisional Patent Application Ser. 62/303,508, filed on Mar. 4, 2016, the entire contents each of which are hereby incorporated by reference in their entirety.

### FIELD OF THE INVENTION

The present invention is in the field of pharmaceutical compositions and discloses stable compositions comprising at least one selective Cannabinoid Receptor Type 2 (“CB2”) receptor agonist wherein the composition is used to treat a patient suffering from a mental disorder.

### BACKGROUND

Mental disorders can arise from multiple sources and affect a large percentage of the population. There are a range of different types of treatment of mental disorders and what is most suitable depends on the disorder and on the individual.

Schizophrenia is a mental disorder which affects about 1% of the population (Lewis & Lieberman, 2000), and genetic and environmental factors underlie the eventual eruption of the disease (Ross, 2006). Schizophrenia is often chronic, characterized by deterioration of social contact, cognitive deficits, anxiety and depression, resulting in suicide in about 10% of the schizophrenic population (Lewis & Lieberman, 2000).

Different subtypes of schizophrenia are defined according to the most significant and predominant characteristics present, as follows: paranoid schizophrenia, disorganized schizophrenia, undifferentiated schizophrenia, catatonic schizophrenia and residual schizophrenia. Onset of schizophrenia can occur at any age, infancy, childhood, adolescence or adulthood.

### SUMMARY

The invention relates to the field of therapy of mental disorders and more particularly, but not exclusively, to compositions comprising at least one Cannabinoid Receptor Type 2 (CB2) selective or highly selective receptor agonist and optionally at least one additional antipsychotic agent, methods of making the compositions and methods of treatment using same for the treatment of mental disorders.

Compositions and methods of treatment of mental disorders are disclosed therein.

According to aspects illustrated therein, there is provided a stabilized composition comprising at least one Cannabinoid Receptor Type 2 (CB2) receptor selective agonist in substantially pure form, optionally at least one additional antipsychotic agent and a pharmaceutically acceptable carrier.

Some aspects of the invention relate to compositions comprising CB2 receptor selective or highly selective agonists, such as HU-308 ([(1R,2R,5R)-2-[2,6-dimethoxy-4-(2-methyloctan-2-yl)phenyl]-7,7-dimethyl-4-bicyclo[3.1.1]hept-3-enyl]methanol), as sole active agent, and optionally at least one antipsychotic agent, methods of making the compositions and methods using the compositions for the treatment of mental disorders.

Some other aspects of the invention relate to compositions comprising as CB2 receptor selective agonist beta-caryophyllene (BCP) and optionally at least one antipsychotic agent, methods of making the compositions and methods using the compositions for the treatment of mental disorders.

Some other aspects of the invention relate to the effect of the compositions on hepatic cytochrome P450 system. On the one hand the concomitant use of an antipsychotic and active agents that are competitively metabolized by the same system may cause a potentially harmful drug-drug interaction; drug interactions may also change how medications work or increase risk for serious side effects. On the other hand, a drug that can block CYP enzymatic inhibition can reduce the toxicity of an antipsychotic.

According to an aspect of the invention, there is provided a stabilized composition comprising beta-caryophyllene (BCP) and/or HU-308 and a pharmaceutically effective carrier for use in treating mental disorders, one of which is schizophrenia. According to an aspect of the invention, there is provided a stabilized composition comprising beta-caryophyllene (BCP), HU-308, their metabolites, analogs, or derivatives thereof, and a pharmaceutically effective carrier for use in treating mental disorders, one of which is schizophrenia. In some aspects, the composition is for use in the treatment of a human subject. In some other aspects, the composition is for use in the treatment of a non-human subject.

According to an aspect of the invention, there is provided a stabilized composition comprising an antioxidant, beta-caryophyllene (BCP) and/or HU-308 and a pharmaceutically effective carrier for use in treating mental disorders, one of which is schizophrenia. In some aspects, the composition is for use in the treatment of a human subject. In some other aspects, the composition is for use in the treatment of a non-human subject.

The schizophrenia can be paranoid schizophrenia, disorganized schizophrenia, undifferentiated schizophrenia, catatonic schizophrenia and residual schizophrenia. Onset of schizophrenia can occur at any age, infancy, childhood, adolescence or adulthood.

In some embodiments, the treatment comprises treating at least one symptom of schizophrenia selected from the group consisting of a negative symptom of schizophrenia, and/or a positive symptom of schizophrenia, as well as other symptoms of schizophrenia (e.g. cognitive symptoms).

In some aspects of the invention, the pharmaceutically effective carrier comprises dimethyl sulfoxide (DMSO). In some other aspects, the pharmaceutically effective carrier comprises DMSO, saline and Cremophor EL. In some aspects of the invention, the pharmaceutically effective carrier comprises DMSO, saline and Cremophor EL at a ratio of about 1:0.6:18.4 Cremophor EL:DMSO:saline.

In some aspects, the pharmaceutically effective carrier comprises ethanol, saline and Cremophor EL at a ratio of about 1:0.6:18.4 Cremophor EL:ethanol:saline.

In some aspects of the invention, the composition is formulated as an injectable solution dosage form. The injectable solution is formulated to be administered by a route selected from the group consisting of intravenous injection, intramuscular injection, intradermal injection, intraperitoneal injection, intrathecal injection, depot injection, subcutaneous injection, intra-arterial injection and injectable suspension, according to case.

In some aspects of the invention, the composition is formulated as an orally-administrable dosage form. The composition is formulated in a dosage form selected from the group consisting of a tablet, sublingual tablet, caplet, depot, transdermal gel, cream, topical spray, nasal spray, transdermal patch, spray, suppository, chewable, capsule, dragee, powder, granules, suspension, solution, emulsion, syrup, transmucosal, lozenge, sachet, gastroresistant oral dosage, gastroresistant softgel capsule, sprinkle and an ingestible solution.

In some aspects of the invention, the composition further comprises at least one additional antipsychotic agent. The at least one additional antipsychotic agent can be selected from the group consisting of benperidol, bromperidol, droperidol, haloperidol, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, amisulpride, amoxapine, aripiprazole, dehydroaripiprazole, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, brexpiprazole, ITI-007, pimavanserin, RP5063 (RP5000) cannabidiol (CBD), cannabidivarin (CBDV), cannabiodiolic acid (CBDA), tetrahydrocannabivarin (THCV), OPC-14857, DM-1458, DM-1451, DM-1452, DM-1454, DCPP, cannabigerol (CBG) and its analogs CBGA and CBGV and combinations thereof.

According to some aspects of the invention, there is provided the use of beta-caryophyllene (BCP) and/or HU-308 and a pharmaceutically effective carrier in the manufacture of a composition (also known as a medicament) for treating schizophrenia in a subject in need thereof. In some aspects, the composition is formulated for use in the treatment of a human subject. In some other aspects, the composition is formulated for use in the treatment of a non-human subject.

In some aspects, the schizophrenia can be paranoid schizophrenia, disorganized schizophrenia, undifferentiated schizophrenia, catatonic schizophrenia and residual schizophrenia.

In some aspects, the pharmaceutically effective carrier comprises dimethyl sulfoxide (DMSO). The pharmaceutically effective carrier comprises DMSO, saline and Cremophor EL. In some aspects, the pharmaceutically effective carrier comprises DMSO, saline and Cremophor EL at a ratio of about 1:0.6:18.4 Cremophor EL:DMSO:saline.

In some aspects, the pharmaceutically effective carrier comprises ethanol, saline and Cremophor EL at a ratio of about 1:0.6:18.4 Cremophor EL:ethanol:saline.

In some aspects, a single discrete unit (e.g., a single tablet, capsule, metered liquid) of the composition can be is manufactured comprising BCP at a weight in the range of from about 1 mg to about 1000 mg.

In some aspects, the composition is formulated as an injectable solution dosage form. The injectable solution is formulated to be administered by a route selected from the group consisting of intravenous injection, intramuscular injection, intradermal injection, intraperitoneal injection, intrathecal injection, subcutaneous injection, intra-arterial injection and injectable suspension, according to case.

In some aspects of the invention, the composition is formulated as an orally-administrable dosage form. The dosage form is selected from the group consisting of a tablet, sublingual tablet, caplet, depot, transdermal gel, cream, topical spray, nasal spray, transdermal patch, spray, suppository, chewable, capsule, dragee, powder, granules, suspension, solution, emulsion, syrup, transmucosal, lozenge, sachet, gastroresistant oral dosage, gastroresistant softgel capsule, sprinkle and an ingestible solution.

In other aspects, the composition further comprises at least one antipsychotic agent. The at least one antipsychotic agent is selected from the group consisting of benperidol, bromperidol, droperidol, haloperidol, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, amisulpride, amoxapine, aripiprazole, dehydroaripiprazole, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, brexpiprazole, ITI-007, pimavanserin, RP5063 (RP5000) cannabidiol (CBD), cannabidivarin (CBDV), cannabiodiolic acid (CBDA), tetrahydrocannabivarin (THCV), OPC-14857, DM-1458, DM-1451, DM-1452, DM-1454, DCPP, cannabigerol (CBG) and its analogs CBGA and CBGV and combinations thereof.

According to some aspects of the present invention, there is provided a method for treating schizophrenia in a subject in need thereof, the method comprising administering to a patient in need thereof a therapeutically effective dose of a composition comprising beta-caryophyllene (BCP) and a pharmaceutically effective carrier. In some aspects of the invention, the subject is a human subject. In other aspects, the subject is a non-human subject.

In some aspects, the schizophrenia can be paranoid schizophrenia, disorganized schizophrenia, undifferentiated schizophrenia, catatonic schizophrenia, and residual schizophrenia.

In some aspects of the invention, the treatment comprises treating at least one symptom of schizophrenia selected from the group consisting of a negative symptom of schizophrenia and a positive symptom of schizophrenia.

In some aspects, the pharmaceutically effective carrier comprises dimethyl sulfoxide (DMSO). In some aspects, the pharmaceutically effective carrier comprises DMSO, saline and Cremophor EL. The pharmaceutically effective carrier comprises DMSO, saline and Cremophor EL at a ratio of about 1:0.6:18.4 Cremophor EL:DMSO:saline.

In some aspects, the pharmaceutically effective carrier comprises ethanol. In some aspects the pharmaceutically effective carrier comprises ethanol, saline and Cremophor EL. The pharmaceutically effective carrier comprises ethanol, saline and Cremophor EL at a ratio of about 1:0.6:18.4 Cremophor EL:ethanol:saline.

In other aspects, the pharmaceutically effective carrier comprises an antioxidant or free radical scavenger, which can be selected from vitamin E, tocopherols, tocopherol esters, vitamin C, beta-carotene, butylated hydroxy toluene, butylated hydroxyanisole or other FDA-approved antioxidant listed in the FDA's Inactive Ingredients Database (IID). The antioxidant is not DMSO or ethanol.

In some embodiments, the ratio of antioxidant/CB2 receptor agonist is from 1:1 to 2:1 w/w. In some embodiments, ratio of antioxidant/CB2 receptor agonist is from 1:1 to 3:1 w/w. In some embodiments, the ratio of antioxidant/CB2 receptor agonist is from 1:1 to 4:1 w/w. In some embodiments, the ratio of antioxidant/CB2 receptor agonist is from 1:1 to 5:1 w/w. In some embodiments, the ratio of antioxidant/CB2 receptor agonist is from 2:1 to 3:1 w/w. In some embodiments, the ratio of antioxidant/CB2 receptor agonist is from 2:1 to 4:1 w/w. In some embodiments, the ratio of antioxidant/CB2 receptor agonist is from 2:1 to 5:1 w/w. In some embodiments, the ratio of antioxidant/CB2 receptor agonist is from 3:1 to 4:1 w/w. In some embodiments, the ratio of antioxidant/CB2 receptor agonist is from 3:1 to 5:1 w/w. In some embodiments, the ratio of antioxidant/CB2 receptor agonist is from 1:1 to 10:1 w/w. In some embodiments, the ratio of antioxidant/CB2 receptor agonist is from 2:1 to 10:1 w/w. In some embodiments, the ratio of antioxidant/CB2 receptor agonist is from 3:1 to 10:1 w/w. In some embodiments, the ratio of antioxidant/CB2 receptor agonist is from 4:1 to 10:1 w/w. In some embodiments, the ratio of antioxidant/CB2 receptor agonist is from 5:1 to 10:1 w/w. In some embodiments, the ratio of antioxidant/CB2 receptor agonist is from 6:1 to 10:1 w/w. In some embodiments, the ratio of antioxidant/CB2 receptor agonist is from 7:1 to 10:1 w/w. In some embodiments, the ratio of antioxidant/CB2 receptor agonist is from 8:1 to 10:1 w/w. In some embodiments, the ratio of antioxidant/CB2 receptor agonist is from 9:1 to 10:1 w/w. In some embodiments, the ratio of antioxidant/CB2 receptor agonist is from 5:1 to 15:1 w/w. In some embodiments, the ratio of antioxidant/CB2 receptor agonist is from 5:1 to 20:1 w/w. In some embodiments, the ratio of antioxidant/CB2 receptor agonist is from 5:1 to 25:1 w/w. In some embodiments, the ratio of antioxidant/CB2 receptor agonist is from 5:1 to 30:1 w/w. In some embodiments, the ratio of antioxidant/CB2 receptor agonist is from 5:1 to 35:1 w/w. In some embodiments, the ratio of antioxidant/CB2 receptor agonist is from 5:1 to 40:1 w/w. In some embodiments, the ratio of antioxidant/CB2 receptor agonist is from 10:1 to 15:1 w/w. In some embodiments, the ratio of antioxidant/CB2 receptor agonist is from 10:1 to 20:1 w/w. In some embodiments, the ratio of antioxidant/CB2 receptor agonist is from 10:1 to 25:1 w/w. In some embodiments, the ratio of antioxidant/CB2 receptor agonist is from 10:1 to 30:1 w/w. In some embodiments, the ratio of antioxidant/CB2 receptor agonist is from 10:1 to 35:1 w/w. In some embodiments, the ratio of antioxidant/CB2 receptor agonist is from 10:1 to 40:1 w/w. In some embodiments, the ratio of antioxidant/CB2 receptor agonist is from 15:1 to 20:1 w/w. In some embodiments, the ratio of antioxidant/CB2 receptor agonist is from 15:1 to 25:1 w/w. In some embodiments, the ratio of antioxidant/CB2 receptor agonist is from 15:1 to 30:1 w/w. In some embodiments, the ratio of antioxidant/CB2 receptor agonist is from 15:1 to 35:1 w/w. In some embodiments, the ratio of antioxidant/CB2 receptor agonist is from 15:1 to 40:1 w/w. In some embodiments, the ratio of antioxidant/CB2 receptor agonist is from 20:1 to 25:1 w/w. In some embodiments, the ratio of antioxidant/CB2 receptor agonist is from 20:1 to 30:1 w/w. In some embodiments, the ratio of antioxidant/CB2 receptor agonist is from 20:1 to 35:1 w/w. In some embodiments, the ratio of antioxidant/CB2 receptor agonist is from 20:1 to 40:1 w/w. In some embodiments, the ratio of antioxidant/CB2 receptor agonist is from 25:1 to 30:1 w/w. In some embodiments, the ratio of antioxidant/CB2 receptor agonist is from 25:1 to 35:1 w/w. In some embodiments, the ratio of antioxidant/CB2 receptor agonist is from 25:1 to 40:1 w/w. In some embodiments, the ratio of antioxidant/CB2 receptor agonist is from 30:1 to 35:1 w/w. In some embodiments, the ratio of antioxidant/CB2 receptor agonist is from 30:1 to 40:1 w/w.

In some embodiments, the above composition can spontaneously form an oil-in-water emulsion upon dilution with water containing media or body fluid. In some aspects of the invention, the administration comprises injecting the composition to the patient in need thereof. The injecting route can be selected from the group consisting of intravenous injection, intramuscular injection, intradermal injection, intraperitoneal injection, intrathecal injection, subcutaneous injection, intra-arterial injection and injectable suspension according to case.

In other aspects of the invention, the administering comprises orally administering the composition to the subject.

In some aspects, the method can further comprise co-administering at least one antipsychotic agent. In some embodiments, the at least one antipsychotic agent can be selected from the group consisting of benperidol, bromperidol, droperidol, haloperidol, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, amisulpride, amoxapine, aripiprazole, dehydroaripiprazole, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, brexpiprazole, ITI-007, pimavanserin, RP5063 (RP5000) cannabidiol (CBD), cannabidivarin (CBDV), cannabiodiolic acid (CBDA), tetrahydrocannabivarin (THCV), OPC-14857, DM-1458, DM-1451, DM-1452, DM-1454, DCPP, cannabigerol (CBG) and its analogs CBGA and CBGV and combinations thereof.

In some aspects of the invention, the at least one antipsychotic agent can be co-administered in a single dosage form together with the BCP. In some other aspects, the at least one antipsychotic agent is co-administered in a dosage form separate from the CB2 receptor selective agonist. The co-administration can comprise sequential or simultaneous administration. The sequential administration can comprise administration of the at least one antipsychotic agent prior to administration of the CB2 receptor selective agonist or subsequent to administration of the CB2 receptor selective agonist.

In some embodiments, the CB2 receptor selective agonist can be BCP. When found in nature, BCP typically appears as a mixture of two isomers E-BCP and Z-BCP, together with sesquiterpenes such as alpha-humulene and traces of derivatives such as BCP oxide, copaene, eugenol and δ-cadinene. Typically, natural sources include a greater proportion of E-BCP than Z-BCP.

For implementing the teachings herein, the BCP can include E-BCP and Z-BCP, alone or in combination.

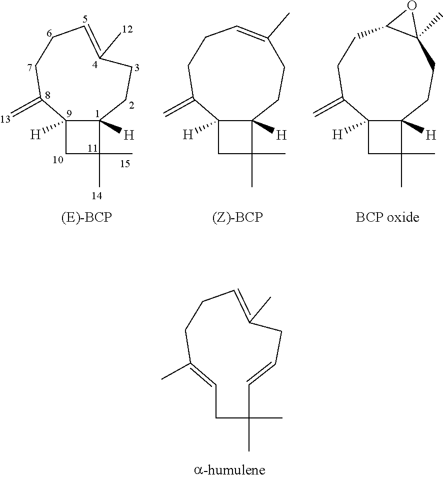

In some aspects, the BCP used for implementing the teachings herein is at least about 65%, at least about 75%, at least about 85% and even at least about 95% by weight E-BCP. In some embodiments, the BCP is substantially pure (at least about 98% or about 99% by weight) E-BCP.

In some aspects, the BCP used for implementing the teachings herein is at least about 65%, at least about 75%, at least about 85% and even at least about 95% by weight Z-BCP. In some embodiments, the BCP is substantially pure (at least about 98% or about 99% by weight) Z-BCP.

In some aspects, the BCP used for implementing the teachings herein is at least about 65%, at least about 75%, at least about 85% and even at least about 95% or about 98% by weight E-BCP and/or Z-BCP. In some embodiments, the BCP is substantially pure (at least about 97%, at least about 98%, at least about 99% by weight) E-BCP and/or Z-BCP.

For example, in some aspects, the BCP used for implementing the teachings herein comprises at least about 49% E-BCP, about 1-49% Z-BCP, about 1-5% BCP oxide and about 1-15% alpha humulene.

For example, in some aspects, the BCP used for implementing the teachings herein comprises about 45-49% E-BCP, about 45-49% Z-BCP, about 1-5% BCP oxide and about 1-5% alpha humulene.

For example, in some aspects BCP used for implementing the teachings herein comprises about 45-90% E-BCP, about 5-30% Z-BCP, about 1-5% BCP oxide and traces alpha humulene.

According to some aspects of the invention, there is provided a composition comprising a CB2 receptor selective agonist and a pharmaceutically effective carrier for use in treating schizophrenia.

According to some aspects of the invention, there is provided a use of a composition comprising a CB2 receptor selective agonist and a pharmaceutically effective carrier in the manufacture of a composition for treating schizophrenia in a subject in need thereof.

According to some aspects of the invention, there is provided a method for the treating schizophrenia in a subject in need thereof, the method comprising administering a therapeutic composition comprising a CB2 receptor selective agonist and a pharmaceutically effective carrier.

Any suitable CB2 receptor selective agonist may be used in implementing the composition, the use or the method of treatment. In some embodiments, the CB2 receptor selective agonist is BCP and/or HU-308.

In some aspects, the teachings herein are applied to the treatment of human subjects, for example, humans suffering from schizophrenia.

In some other aspects, the teachings herein are applied to the treatment of non-human animal subjects suffering from mental disorder one of which is schizophrenia.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

FIGS. 1A and 1B relate to mouse body weight at PND 16-17: FIG. 1A is a line graph showing changes in body weight at postnatal days 3 to 17 in mice treated with phencyclidine (PCP), PCP+BCP or control (vehicle) and FIG. 1B is a bar graph showing body weight for the 3 groups at postnatal day 17;

FIGS. 2A-2C relate to open field test at PND 16-17: FIGS. 2A and 2B are line graphs showing ambulation (2A) and rearing (2B) at PND 16-17 and FIG. 2C is a bar graph showing body weight at PND 17;

FIGS. 3A-3F relate to open field test at PND 16-17: FIGS. 3A and 3D are bar graphs showing body weight for males (3A) and females (3D), FIGS. 3B and 3E are line graphs showing ambulation in males (3E) and females (3F) and FIGS. 3C and 3F are line graphs showing rearing in males (3C) and females (3F);

FIGS. 4A-4F relate to open field test at PND 35-37: FIGS. 4A and 4D are bar graphs showing body weight for males (4A) and females (4D), FIGS. 4B and 4E are line graphs showing rearing in males (4B) and females (4E) and FIGS. 4C and 4F are line graphs showing ambulation in males (4C) and females (4F);

FIGS. 5A-5D relate to pre-pulse inhibition at age 8 weeks: FIGS. 5A and 5C are bar graphs showing response to startle for 8 week old males (5A) and females (5C); FIGS. 5B and 5D are line graphs showing percentage inhibition of prepulse inhibition for males (5B) and females (5D);

FIGS. 6A-6H relate to elevated plus maze test at age 13 weeks: female duration closed (6A), male duration closed (6B), female duration open (6C), male duration open (6D), female duration distal open (6E), male duration distal open (6F), female open/close duration (6G) and male open/close duration (6H);

FIGS. 7A-7I are bar graphs showing mRNA expression of cannabinoid receptors in 9 day old mice for glyceraldehyde 3-phosphate dehydrogenase (GAPDH) in the left cortex (7A), right cortex (7B) and brain stem (7G); for Cannabinoid Receptor Type 1 (CB1) in the left cortex (7C), right cortex (7D) and brain stem (7H); and for CB2 in the left cortex (7E), right cortex (7F), and brain stem (7I) for control mice and mice treated with PCP;

FIG. 8 is a Table detailing the percentage change (%) relative to control animals of protein expression of cannabinoid receptors in 2 week old mice;

FIGS. 9A-9C are bar graphs showing protein expression of 67 kDa glutamic acid decarboxylase (GAD67)/actin in the left cortex (9A), right cortex (9B) and brain stem (9C) of 2-week old mice treated with saline or PCP;

FIGS. 9D-9F are bar graphs showing protein expression of 67 kDa glutamic acid decarboxylase (GAD67)/actin in the left cortex (9D), right cortex (9E) and brain stem (9F) of 9-day old mice treated with saline or PCP;

FIGS. 10A-10C relate to monoacylglycerol lipase (MGL) expression in 2 week old mice treated with saline or PCP: bar graph relating to the left cortex (10A), bar graph relating to the right cortex (10B) and Western blot (10C);

FIGS. 11A and 11B are schematic representations of the endocannabinoid synthesizing and degrading pathways as described in Anavi-Goffer, ChemBioChem 2009;

FIGS. 12A-12C relate to PND17 using a DMSO-based vehicle: line-graph showing male ambulation (12A), line-graph showing male rearing (12B) and line graph showing male body weight (12C);

FIGS. 13A-13C relate to PND16: line-graph showing body weight over PND 3-17 (13A), line-graph showing male and female ambulation (13B) and line-graph showing male and female rearing (13C);

FIGS. 14A-14E show results demonstrating that BCP treatment at adolescence reversed the effect of PCP on ambulation but did not affect body weight: line graph of body weight at PND 40-68 (14A), bar graph of female and male body weight at PND63 (14B), line graph of male ambulation at PND 63 (14D), line graph of female ambulation at PND 63 and line graph of male and female ambulation at PND 63;

FIGS. 15A-15C show results demonstrating that BCP treatment at adolescence reversed the effect of PCP on rearing: line graph of male and female rearing at PND63 (15A), line graph of male rearing at PND63 (15B) and line graph of female rearing at PND63 (15C);

FIGS. 16A-16C show results demonstrating that BCP treatment at adolescence reversed the effect of PCP on PPI: line graph of % PPI at PND68 (16A); bar graph of female startle response at PND68 (16B) and bar graph of male startle response at PND68 (16C);

FIGS. 17A-17C show results demonstrating that BCP treatment at adolescence reversed the effect of PCP on the response to tone (PPI test): line graph of response to tone at PND68 (17A); line graph of female response to tone at PND68 (17B) and line graph of male response to tone at PND68 (17C);

FIGS. 18A-18C show results demonstrating that BCP treatment at adolescence did not affect the startle response at the end of the PPI test: female startle response at PND 68 (18A), male startle response at PND 68 (18B) and all-mice startle response at PND68 (18C);

FIGS. 19A-19F show results demonstrating that BCP treatment at adolescence did not reverse the effects of PCP in plus maze test: open/close duration at PND 64 (19A), open/(close+open) duration at PND 64 (19B), distal open/(close+open) duration at PND 64 (19C), open/close frequency at PND 64 (19D), open/(open+close) frequency at PND 64 (19E) and distal open/(open+close) frequency at PND 64 (19F);

FIGS. 20A-20E show results demonstrating that BCP treatment at adolescence reversed the effects of PCP on the time spent in the hidden zone (behavior in the Phenotyper cage): bar graph of female hidden zone duration at PND 91 (20A), bar graph of male hidden zone duration at PND 91 (20B), bar graph of male and female hidden zone duration at PND 91 (20C), bar graph of male hidden zone frequency at PND 91 (20D) and bar graph of female hidden zone frequency at PND 91 (20E);

FIGS. 21A-21C show results demonstrating that BCP treatment at adolescence reversed the effects of PCP on frequency of entries to the wheel (motor behavior in the Phenotyper cage: bar graph of female wheel zone frequency at PND 91 (21A), bar graph of male wheel zone frequency at PND 91 (21B) and bar graph of male and female wheel zone frequency at PND 91 (21C);

FIGS. 22A-22F show results demonstrating that BCP treatment at adolescence on the time spent at drinking and food zones (Phenotyper cage): bar graph showing male food zone duration at PND91 (22A), bar graph showing female food zone duration at PND91 (22B), bar graph showing male and female food zone duration at PND91 (22C), bar graph showing male drink zone duration at PND91 (22D), bar graph showing female drink zone duration at PND91 (22E) and bar graph showing male and female drink zone duration at PND91 (22F);

FIGS. 23A-23E show results demonstrating that BCP treatment at adolescence improved exploration and rearing behaviors of male PCP treated mice at PND 104: line graph of female ambulation at PND 104 (23A), line graph of male ambulation at PND 104 (23B), line graph of female rearing at PND 104 (23C), line graph of male rearing at PND 104 (23D) and bar graph of male and female body weight at PND 104 (23E);

FIGS. 24A-24C show results demonstrating that BCP treatment at adolescence did not reverse the effect of PCT on grooming at PND104: bar graph of female grooming at PND 104 (24A), bar graph of male grooming at PND 104 (24B) and bar graph of male and female grooming at PND 104 (24C);

FIGS. 25A-25I show results demonstrating that BCP treatment at adolescence reversed the effect of PCP on attention at PND106 (PPI test): bar graph of female startle at PND 106 (25A), bar graph of male startle at PND 106 (25B), bar graph of male and female startle at PND 106 (25C), line graph of female response to tone of varying intensity (25D), line graph of female response to tone of varying intensity (25E), line graph of female response to tone of varying intensity (25F), line graph of % prepulse inhibition for females at PND 106 (25G), line graph of % prepulse inhibition for males at PND 106 (25H) and line graph of % prepulse inhibition for males and females at PND 106 (25I);

FIGS. 26A-26F show results demonstrating that BCP treatment at adolescence reversed the effect of PCP on frequency of entries to hidden zone (Phenotyper cage) at PND 105: bar graph of male hidden zone frequency at PND 105 (26A), bar graph of female hidden zone frequency at PND 105 (26B), bar graph of male and female hidden zone frequency at PND 105 (26C), bar graph of male wheel zone frequency at PND 105 (26D), bar graph of female wheel zone frequency at PND 105 (26E) and bar graph of male and female wheel zone frequency at PND 105 (26F);

FIGS. 27A-27E show results demonstrating that BCP treatment at adolescence reversed the effect of PCP on time spent at the hidden zone but not the time spent in the wheel zone (Phenotyper cage) at PND 105: bar graph of male hidden zone duration at PND 105 (27A), bar graph of female hidden zone duration at PND 105 (27B), bar graph of male wheel zone duration at PND 105 (27C), bar graph of female wheel zone duration at PND 105 (27D) and bar graph of male and female wheel zone duration at PND 105 (27E); and

FIGS. 28A-28B show results demonstrating that AM630 reversed the effect of BCP on PCP-induced inhibition of ambulation and rearing: line graph of male ambulation at 17 days (28A) and line graph of male rearing at 17 days (28B).

FIGS. 29A-B show results demonstrating that oral treatment with BCP at adolescence reversed the effect of PCP on mice in the open field test (29A) and in the forced swim test (29B).

FIGS. 30A-B show results demonstrating that oral treatment with BCP at adolescence reversed the effect of PCP on male mice in the social interaction test (30A) but did not affect their body weight (30B).

FIG. 31A shows that oral treatment with risperidone at adolescence reversed the effect of PCP on activity in the open field test.

FIG. 31B shows results demonstrating that postnatal treatment with HU-308 reversed the effect of PCP in the PPI test.

FIG. 32 shows the effect of HU-308 in the DOI test. HU-308 significantly reversed the effect of DOI on grooming response.

FIG. 33A shows the risperidone inhibited the activity of CYP2D6 enzyme.

FIG. 33B shows that BCP blocked the effect of risperidone and restored the activity of CYP2D6 enzyme.

FIG. 34 shows that oral treatment with 5 mg/kg BCP in SEDDS formulation reversed the effect of PCP in the open field test.

### DETAILED DESCRIPTION

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof.

As used herein, the indefinite articles “a” and “an” mean “at least one” or “one or more” unless the context clearly dictates otherwise.

As used herein, when a numerical value is preceded by the term “about”, the term “about” is intended to indicate +/−10%.

As used herein, the term “treating” or ‘treatment” includes curing a condition, treating a condition, preventing a condition, treating symptoms of a condition, curing symptoms of a condition, ameliorating symptoms of a condition, treating effects of a condition, ameliorating effects of a condition, and preventing results of a condition

As used herein a “therapeutic composition” refers to a preparation of one or more of the active ingredients with other components such as pharmaceutically-acceptable carriers and excipients. The purpose of a therapeutic composition is to facilitate administration of an active ingredient to a subject.

The term “pharmaceutically acceptable carrier” or “pharmaceutically effective carrier” refers to a carrier or a diluent that does not cause significant irritation to a subject, effectively provides the active agent(s) to the patient in need thereof and does not substantially abrogate the activity and properties of the administered active ingredients. An adjuvant is included under these phrases. The term “excipient” refers to an inert substance added to a therapeutic composition to further facilitate administration of an active ingredient.

Therapeutic compositions used in implementing the teachings herein may be formulated using techniques with which one of average skill in the art is familiar in a conventional manner using one or more pharmaceutically-acceptable carriers comprising excipients and adjuvants, which facilitate processing of the active ingredients into a pharmaceutical composition and generally includes mixing an amount of the active ingredients with the other components. Suitable techniques are described in “Remington's Pharmaceutical Sciences,” Mack Publishing Co., Easton, Pa., latest edition, which is incorporated herein by reference. For example, pharmaceutical compositions useful in implementing the teachings herein may be manufactured by one or more processes that are well known in the art, e.g., but not limited to mixing, blending, homogenizing, dissolving, granulating, emulsifying, encapsulating, entrapping and lyophilizing processes.

Pharmaceutical compositions suitable for implementing the teachings herein include compositions comprising active ingredients in an amount effective to achieve the intended purpose (a therapeutically effective amount). Determination of a therapeutically effective amount is well within the capability of those skilled in the art, for example, is initially estimated from animal models such as rats, mice, monkey or pigs.

The present invention provides a stabilized composition comprising a therapeutically effective dose of at least one CB2 receptor selective or highly selective agonist in substantially pure form of at least about 98% w/w in a pharmaceutically effective carrier and optionally a therapeutically effective dose of at least one antipsychotic agent in a pharmaceutically effective carrier, for use in treating a mental disorder in a patient in need thereof.

The present invention also provides a stabilized composition comprising a therapeutically effective dose of at least one CB2 receptor selective or highly selective agonist of at least 80% w/w with alpha-humulene and traces of BCP oxide, copaene, eugenol and/or δ-cadinene in a pharmaceutically effective carrier and optionally a therapeutically effective dose of at least one antipsychotic agent in a pharmaceutically effective carrier, for use in treating a mental disorder in a patient in need thereof.

The present invention also provides a stabilized composition comprising a therapeutically effective dose of at least one CB2 receptor selective or highly selective agonist, at least one antioxidant and optionally a therapeutically effective dose of at least one antipsychotic agent in a pharmaceutically effective carrier, for use in treating a mental disorder in a patient in need thereof.

The present invention also provides a stabilized composition comprising a therapeutically effective dose of at least one CB2 receptor selective or highly selective agonist, comprising beta-caryophyllene (BCP), HU-308, their metabolites, analogs, derivatives and a pharmaceutically effective carrier for use in treating mental disorders, one of which is schizophrenia.

In the context of this disclosure, the term “selective” when used alone is meant generically, that is it includes also highly selective.

Some of the CB2 receptor selective or highly selective agonists of this invention are synthetic cannabinoids or cannabinoids of plant origin (phytocannabinoids) such as cannabis, hemp, marijuana, cloves, black caraway, hops, basil, oregano, black pepper, lavender, rosemary, cinnamon, malabathrum, ylang-ylang, copaiba oil, etc.

The cannabinoids are a group of chemical compounds of very diverse structures.

The most important types of phytocannabinoids are: cannabigerol-type (CBG), cannabichromene-type (CBC), cannabidiol-type (CBD), tetrahydrocannabinol- and cannabinol-type (THC, CBN), cannabielsoin-type (CBE), iso-tetrahydrocannabinol-type (iso-THC), cannabicyclol-type (CBL), cannabicitran-type (CBT). The most studied cannabinoids are THC, CBD, CBG and CBN. At least 85 different cannabinoids have been isolated from the cannabis plant. These compounds have very different affinities for the cannabinoid or non-cannabinoid receptors—some are neutral ligands (no or very little affinity to the cannabinoid receptors), some are CB1 and CB2 receptor agonists, some are CB1 and CB2 receptor partial agonists, some are CB1 and CB2 receptor antagonists, some are CB1 and CB2 receptor inverse-agonists, some are combination thereof and only a few are specific and selective agonists or antagonists. Some cannabinoids (like CBD, CBDA, CBDV, CBG, CBGA, CBGV, THC and THCV) are inhibitors of the GPR55 ligand (Anavi-Goffer et al. 2012).

THC, THCV and CBN are non-selective CB1 and CB2 receptor ligands. In fact delta-9-THC is a weak CB1 and CB2 receptor partial agonist (Childers, 2006), thus that in the presence of a more potent selective agonist delta-9-THC will antagonize its effects. CBC, CBD, CBDV, CBDA, CBG, CBGV, CBGA, THCA and THCV have not been reported to activate the CB1 or CB2 receptors with significant potency (Handbook of Cannabis, Oxford University Press, R. G. Pertwee Editor, p. 137, 2014). Summing up, unlike the CB2 receptor selective agonists of this invention, none of the above cannabinoids are selective or highly selective CB2 receptor agonists.

Most of the commercially available cannabinoids are in fact loosely defined mixtures of a cannabinoid with other cannabinoids, impurities, geometrical isomers and enantiomers. The cannabinoid's proneness to spontaneous oxidation complicates even more the purity issue of these substances.

The affinities for two different cannabinoid receptors (CB1 and CB2 receptors) complicate the issue of pharmacological activity. Moreover, postnatal CB2 receptor expression is higher than in adults. In fact, CB2 receptors are largely absent in the central nervous system (CNS) of adult mammals under normal conditions. Therefore, the present disclosure uses CB2 receptor selective agonists at specific doses according to age. Therefore, the present disclosure uses as active agents well-defined stabilized highly pure CB2 receptor selective agonists. Most of the CB2 selective agonists of this invention are potent selective CB2 receptor agonists.

The mental disorder of this invention is selected from the group consisting of schizophrenia, bipolar disorder I and II, unipolar disorder, multiple personality disorder, psychotic disorders, depression, psychotic depression, depressive disorders, major depressive disorder, stereotypic movement disorder, autism spectrum disorders, obsessive-compulsive disorder (OCD), bacterial-induced tic disorder, pediatric autoimmune neuropsychiatric disorders associated with streptococcal infections (PANDAS), chorea (Sydenham's chorea (SC), chorea minor, chorea gravidarum, drug-induced chorea), drug-induced repetitive behaviors, akathisia, dyskinesias, Wernicke-Korsakoff syndrome, Tourette's syndrome, tic disorders, epilepsy, anxiety disorders, autistic spectrum disorder, enuresis, addiction, withdrawal symptoms associated with addiction, Asperger syndrome, oppositional defiant disorder, behavioral disturbance, agitation, psychosis/agitation associated with Alzheimer's disease, psychosis associated with Parkinson's disease, psychosis associated with drug of abuse, psychosis associated with psychedelic drug abuse, LSD (lysergic acid diethylamide)—induced psychosis, steroid-induced schizophrenia, steroid-induced psychosis, Capgras syndrome; Fregoli syndrome; Cotard syndrome, personality disorders, borderline personality disorder, avoidant personality disorder, attention-deficit/hyperactive disorder (ADHD, ADD, HD), mania, dementia, anorexia, anorexia nervosa, anxiety, generalized anxiety disorder, social anxiety disorder, body dismographic disorder, obsessive compulsive disorder, paranoid disorder, nightmares, agitation, post-traumatic stress disorder (PTSD), severe mood dysregulation, mental disorder such as depression or anxiety that leads to metabolic diseases such as obesity, depression associated with any of the above clinical conditions and cognitive deficits associated with any of the above clinical conditions.

Some embodiments of the invention relate to compositions comprising at least one Cannabinoid Receptor Type 2 (CB2) receptor selective agonist as sole active, methods of making the compositions and methods using CB2 receptor selective agonists for the treatment of mental disorders.

Some other embodiments relate to compositions comprising Cannabinoid Receptor Type 2 (CB2) receptor selective agonists in combination with at least one antipsychotic agent in a pharmaceutically effective carrier.

Some other embodiments relate to compositions comprising Cannabinoid Receptor Type 2 (CB2) receptor selective agonists in combination with at least one antioxidant and optionally at least antipsychotic agent in a pharmaceutically effective carrier.

Other embodiments of the invention relate to compositions comprising beta-caryophyllene (BCP) as sole CB2 receptor selective agonist, methods of making the compositions and methods using BCP for the treatment of schizophrenia. The use of BCP in schizophrenia is rather unexpected and surprising, as cannabinoids are known to cause aggravation of psychosis in patients with schizophrenia. Thus, for example, THC is known to induce a range of positive symptoms of schizophrenia, and THC treated schizophrenic patients experienced an exacerbation of symptoms (Deepak Cyril D'Souza et al, Eur Arch Psychiatry Clin Neurosci. 2009 October; 259(7): 413-431).

Other embodiments of the invention relate to compositions comprising HU-308 as sole CB2 receptor selective agonist or with combination with BCP, methods of making the compositions and methods using HU-308/BCP mixtures for the treatment of schizophrenia. The use of HU-308 for the treatment of schizophrenia is rather unexpected and surprising, as HU-308 was shown to be a non-psychoactive agent in vivo (Hanus et al, Proc Natl Acad Sci USA 1999; 96(25):14228-33). This publication has set the view that CB2 selective agonists have no effect on the brain. Thus the effect of HU-308 on schizophrenia-like symptoms was unexpected and surprising.

Some other embodiments of the invention relate to compositions comprising beta-caryophyllene (BCP) and/or HU-308 in combination with risperidone, paliperidone, paliperidone palmitate, aripiprazole, quetiapine, CBD and its analogs, THCV, brexpiprazole and combinations thereof, methods of making the compositions and methods using this combination for the treatment of schizophrenia.

Some other embodiments of the invention relate to the effect of the compositions on the hepatic cytochrome P450 system. An unexpected finding of this invention is that BCP has a very small inhibitory effect on CYP2D6 (about 0-20% inhibition). Based on this, we hereby disclose a combination of BCP (a new antipsychotic, according to this invention), with one of the well-known antipsychotic drugs that inhibit CYP2D6 having an improved therapeutic effect as compared to the treatment with the well-known antipsychotic alone.

The Cannabinoid Receptor Type 2 (CB2) is a guanine nucleotide-binding protein (G protein)—coupled receptor that in humans is encoded by the CNR2 gene.

Recent studies have identified the cannabinoid CB2 receptor in the brain. Up-regulation of CB2 receptor expression in the brain during central nervous system pathologies has been demonstrated for certain diseases. Another surprising and unexpected finding was the reduction of monomers of CB2 receptor expression in the brain of schizophrenia while finding an increase of putative CB2 receptors complexes (unidentified yet protein X, or possibility of CB2 receptor-CB2 receptor complexes) in specific brain areas in schizophrenia.

The CB2 receptor selective agonist in the compositions of this invention is selected from the group comprising BCP, HU-308, HU-433, HU-910, HU-914, CB 65, GP 1a, GP 2a, GW 405833, JWH 015, JWH 133, AM1241, L-759,656, L-759,633, MDA 19, SER 601, BML-190, N-alkylamide, rutamarin, diindolylmethane (DIM), cannabilactones, AM1714, AM1710 and combinations thereof.

Beta-caryophyllene (trans-(1R,9S)-8-methylene-4,11,11trimethylbicyclo[7.2.0]undec-4-ene, BCP, CAS 87-44-5) is a CB2-receptor selective agonist (Gertsch et al. 2008). BCP exhibits chirality at positions 1 and 9 and is the 1R,9S enantiomer, the (−) form.

HU-308 ([(1R,2R,5R)-2-[2,6-dimethoxy-4-(2-methyloctan-2-yl)phenyl]-7,7-dimethyl-4-bicyclo[3.1.1]hept-3-enyl]methanol) is a synthetic cannabinoid, which is highly selective for the CB2 receptor.

The fact that orally-administered BCP is absorbed by the digestive tract and becomes systemically available and its apparent substantial non-toxicity makes BCP attractive as a potential active pharmaceutical ingredient. Another surprising and unexpected finding was that oral administration of BCP affects the brain and CNS-controlled behavior.

However, BCP whose main commercial use is as food additive, is not commercially available in pharmaceutical grade. The food additive grade contains a relatively low percentage of BCP, contains impurities like BCP oxide, alpha-humulene and BCP (+) enantiomer and is not well defined analytically.

According to Chicca A. et al Chem.Biol. 2014, 9, 1499-1507, BCP-oxide and alpha-humulene's inactivity suggests the existence of a specific sesquiterpene pharmacophore for CB2 receptor binding in BCP only but not in BCP-oxide and alpha-humulene.

The BCP impurities have potential effects on the therapeutic effect of the compositions of this invention. For example, alpha-humulene is a skin, eyes and respiratory irritant, according to its MSDS.

Also, BCP oxide was found to be an allergen (Skold M, Karlberg A T, Matura M, Borje A, Food Chem Toxicol. 2006 April; 44(4):538-45).

Due to the above side-effects, some compositions of this invention use BCP in substantially pure form E/Z isomers of at least about 98% w/w, being substantially free of BCP oxide and alpha-humulene impurities. Some other compositions use the BCP as mixture with humulene and other minor components, in order to investigate a possible potentiation effects.

One of the drawbacks of BCP is its proneness to autoxidation. Beta-caryophyllene starts to oxidize immediately when air exposed and after 5 weeks almost 50% of the original compound is consumed. Caryophyllene oxide was found to be the major oxidation product ((Skold M, Karlberg A T, Matura M, Borje A, Food Chem Toxicol. 2006 April; 44(4):538-45)). The practical effect of this instability is that conventional compositions containing the compounds have relatively short shelf lives, thus making commercial distribution and storage difficult.

In order to maintain the purity, stability and the therapeutic activity, the compositions of this invention comprising BCP and/or other CB2 receptor selective agonists are stabilized and/or therapeutically enhanced by addition of an antioxidant and/or free-radical scavenger.

Elevated-levels of vitamin E have been found in schizophrenic patients. Vitamin E and the related tocopherols are known antioxidants, used in the compositions of the present disclosure. Therefore, another surprising result of this invention is that the addition of vitamin E to the self-emulsifiable compositions did not block the effect of BCP rather enhanced it. The surprising finding is that vitamin E leads to an enhancement of the anti-schizophrenic activity of CB2 receptor agonists, with special reference to BCP. Thus, oral administration of BCP together with an antioxidant improves the behaviour of subjects having schizophrenia vs. control devoid of antioxidant in vivo.

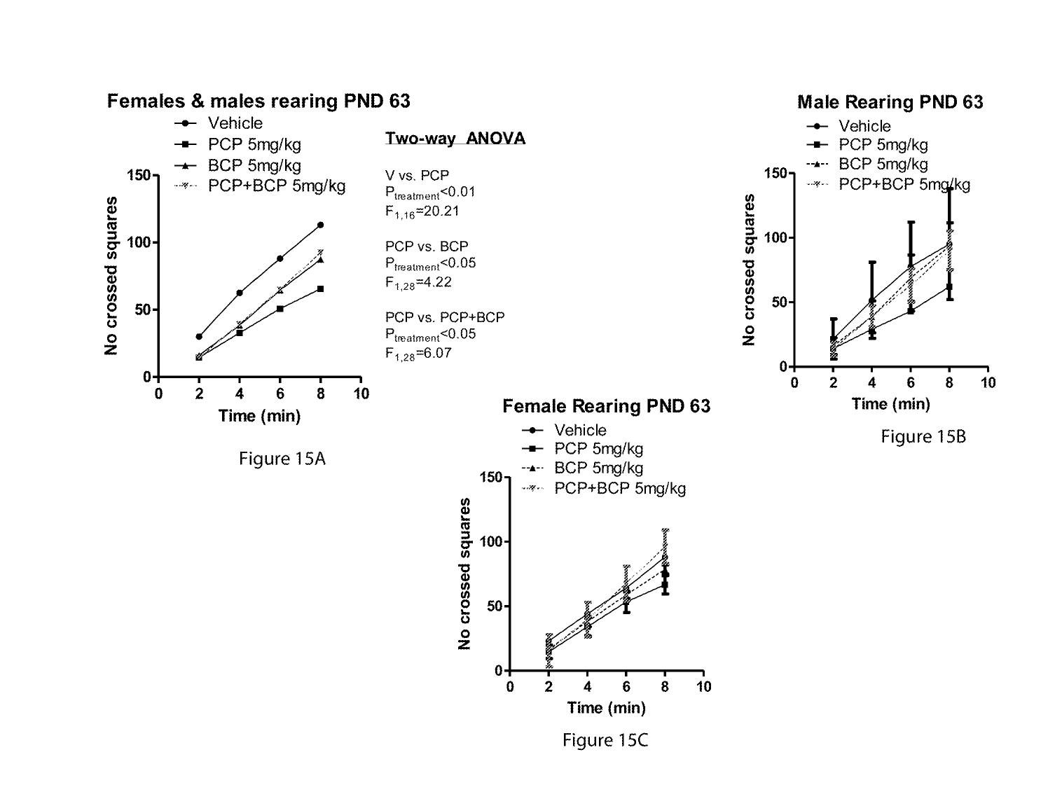

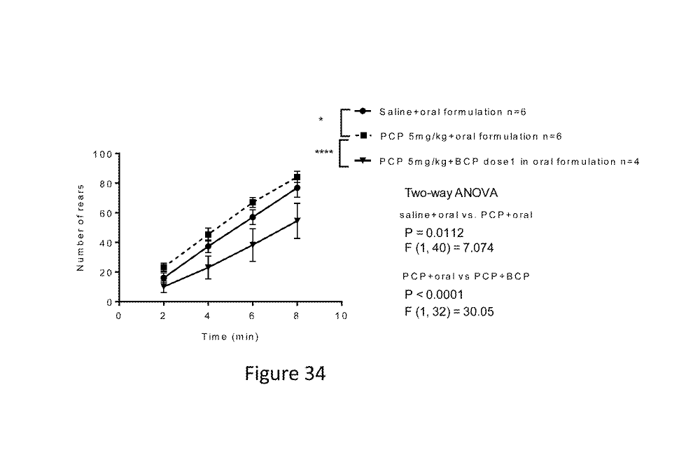

The surprising and unexpected finding was that a combination of BCP with an antioxidant appeared to enhance the reversal effect of BCP. The implication is that compositions with vitamin E are expected to enhance the PCP effects in a murine model of schizophrenia. The results show that the effect of PCP after gavage injections with SEDDS (FIG. 34) was smaller relatively to the effect by intraperitoneal injections (FIGS. 15A-C). Even more surprising was the effect of BCP in SEDDS. The results also show that the effect of BCP in SEDDS was even greater than the performance of the control group (compare the effect of BCP in SEDDS vs. control with SEDSS in FIG. 34 vs. FIG. 15A-C BCP with no antioxidant). Thus, the combination of BCP and an antioxidant has unexpected and enhanced therapeutic effect this enables reducing the dose of BCP for treatment of mental diseases.

In some aspects of the invention, the antioxidant and the selective Cannabinoid Receptor Type 2 agonist are administered in a ratio of from 0.5:1 w/w to 1:0.5 w/w to 1:1 w/w, from about 1:1 to 2:1, from about 2:1 to 5:1 w/w from about 5:1 to 10:1 w/w, from about 10:1 to 20:1 w/w, from about 20:1 to 30:1 w/w, from about 30:1 to 40:1 w/w ratio of antioxidant/s to selective Cannabinoid Receptor Type 2 agonist. The antioxidant may be selected from vitamin E, tocopherols, tocopherol esters, vitamin C, beta-carotene, butylated hydroxy toluene, butylated hydroxyanisole or other FDA-approved antioxidant listed in the FDA's Inactive Ingredients Database (IID). The antioxidant is not DMSO or ethanol.

In some embodiments, the ratio of antioxidant/CB2 receptor agonist is from 1:1 to 2:1 w/w. In some embodiments, ratio of antioxidant/CB2 receptor agonist is from 1:1 to 3:1 w/w. In some embodiments, the ratio of antioxidant/CB2 receptor agonist is from 1:1 to 4:1 w/w. In some embodiments, the ratio of antioxidant/CB2 receptor agonist is from 1:1 to 5:1 w/w. In some embodiments, the ratio of antioxidant/CB2 receptor agonist is from 2:1 to 3:1 w/w. In some embodiments, the ratio of antioxidant/CB2 receptor agonist is from 2:1 to 4:1 w/w. In some embodiments, the ratio of antioxidant/CB2 receptor agonist is from 2:1 to 5:1 w/w. In some embodiments, the ratio of antioxidant/CB2 receptor agonist is from 3:1 to 4:1 w/w. In some embodiments, the ratio of antioxidant/CB2 receptor agonist is from 3:1 to 5:1 w/w. In some embodiments, the ratio of antioxidant/CB2 receptor agonist is from 1:1 to 10:1 w/w. In some embodiments, the ratio of antioxidant/CB2 receptor agonist is from 2:1 to 10:1 w/w. In some embodiments, the ratio of antioxidant/CB2 receptor agonist is from 3:1 to 10:1 w/w. In some embodiments, the ratio of antioxidant/CB2 receptor agonist is from 4:1 to 10:1 w/w. In some embodiments, the ratio of antioxidant/CB2 receptor agonist is from 5:1 to 10:1 w/w. In some embodiments, the ratio of antioxidant/CB2 receptor agonist is from 6:1 to 10:1 w/w. In some embodiments, the ratio of antioxidant/CB2 receptor agonist is from 7:1 to 10:1 w/w. In some embodiments, the ratio of antioxidant/CB2 receptor agonist is from 8:1 to 10:1 w/w. In some embodiments, the ratio of antioxidant/CB2 receptor agonist is from 9:1 to 10:1 w/w. In some embodiments, the ratio of antioxidant/CB2 receptor agonist is from 5:1 to 15:1 w/w. In some embodiments, the ratio of antioxidant/CB2 receptor agonist is from 5:1 to 20:1 w/w. In some embodiments, the ratio of antioxidant/CB2 receptor agonist is from 5:1 to 25:1 w/w. In some embodiments, the ratio of antioxidant/CB2 receptor agonist is from 5:1 to 30:1 w/w. In some embodiments, the ratio of antioxidant/CB2 receptor agonist is from 5:1 to 35:1 w/w. In some embodiments, the ratio of antioxidant/CB2 receptor agonist is from 5:1 to 40:1 w/w. In some embodiments, the ratio of antioxidant/CB2 receptor agonist is from 10:1 to 15:1 w/w. In some embodiments, the ratio of antioxidant/CB2 receptor agonist is from 10:1 to 20:1 w/w. In some embodiments, the ratio of antioxidant/CB2 receptor agonist is from 10:1 to 25:1 w/w. In some embodiments, the ratio of antioxidant/CB2 receptor agonist is from 10:1 to 30:1 w/w. In some embodiments, the ratio of antioxidant/CB2 receptor agonist is from 10:1 to 35:1 w/w. In some embodiments, the ratio of antioxidant/CB2 receptor agonist is from 10:1 to 40:1 w/w. In some embodiments, the ratio of antioxidant/CB2 receptor agonist is from 15:1 to 20:1 w/w. In some embodiments, the ratio of antioxidant/CB2 receptor agonist is from 15:1 to 25:1 w/w. In some embodiments, the ratio of antioxidant/CB2 receptor agonist is from 15:1 to 30:1 w/w. In some embodiments, the ratio of antioxidant/CB2 receptor agonist is from 15:1 to 35:1 w/w. In some embodiments, the ratio of antioxidant/CB2 receptor agonist is from 15:1 to 40:1 w/w. In some embodiments, the ratio of antioxidant/CB2 receptor agonist is from 20:1 to 25:1 w/w. In some embodiments, the ratio of antioxidant/CB2 receptor agonist is from 20:1 to 30:1 w/w. In some embodiments, the ratio of antioxidant/CB2 receptor agonist is from 20:1 to 35:1 w/w. In some embodiments, the ratio of antioxidant/CB2 receptor agonist is from 20:1 to 40:1 w/w. In some embodiments, the ratio of antioxidant/CB2 receptor agonist is from 25:1 to 30:1 w/w. In some embodiments, the ratio of antioxidant/CB2 receptor agonist is from 25:1 to 35:1 w/w. In some embodiments, the ratio of antioxidant/CB2 receptor agonist is from 25:1 to 40:1 w/w. In some embodiments, the ratio of antioxidant/CB2 receptor agonist is from 30:1 to 35:1 w/w. In some embodiments, the ratio of antioxidant/CB2 receptor agonist is from 30:1 to 40:1 w/w.

As used herein, term “stable” means that the quantity of each active ingredient in the composition does not significantly change over the time, during the entire shelf-life of the composition, namely for at least 3 months, advantageously for at least 6 months, more advantageously for at least 12 months, even more advantageously for at least 24 months, wider standard conditions, in particular at a temperature ranging for about 20° C. to 40° C. and a relative humidity ranging for about 30% to 75%. In particular, caryophyllene oxide level is less than about 5% by weight, based on the total weight on the composition, during the entire shelf life of the composition. In the present invention, the composition is advantageously stable during 6 months to 1 year or during 1 year to 2 years under standard conditions.

In some embodiments, compositions comprising BCP and/or other CB2 receptor selective agonists and further comprising an antioxidant, a free-radical scavenger or a combination of an antioxidant and a free-radical scavenger have an extended shelf-life. In some embodiments, the stable or stabilized compositions have the property to lose less than about 5% of the original compound when stored at room temperature from about one year to about two years. In some embodiments, the stable or stabilized compositions have the property to loose less than about 10% of the original compound when stored at room temperature from about one year to about two years. In some embodiments, the stable or stabilized compositions have the property to loose less than about 4% of the original compound when stored at room temperature from about one year to about two years. In some embodiments, the stable or stabilized compositions have the property to lose less than about 3% of the original compound when stored at room temperature from about one year to about two years. In some embodiments, the stable or stabilized compositions have the property to loose less than about 2% of the original compound when stored at room temperature from about one year to about two years. In some embodiments, the stable or stabilized compositions have the property to lose less than about 1% of the original compound when stored at room temperature from about one year to about two years. In some embodiments, the stable or stabilized compositions have the property to lose from about 5% to about 10% of the original compound when stored at room temperature from about one year to about two years. In some embodiments, the stable or stabilized compositions have the property to lose from about 1% to about 5% of the original compound when stored at room temperature from about one year to about two years. Another surprising and unexpected finding was that oral administration of BCP with antioxidant improves behaviour of subjects having schizophrenia vs. control in vivo. The role of CB2 receptor selective agonists in general and BCP in particular in the treatment of schizophrenia, has not previously been studied.

The inventors have studied the effect of BCP in various compositions and modes of administration (see Examples 1-13) in a murine model of schizophrenia, produced by administration of the N-methyl-D-aspartic acid (NMDA) antagonist, phenylcyclidine (PCP).

The inventors have studied the effect of HU-308 in the same murine model of schizophrenia. The results of these studies are detailed in Examples 14-19.

Administration of phencyclidine to rats (e.g. Josselyn and Vaccarino, 1998; Wang & Johnson, 2005; Ballmaier, 2007; Takahashi, 2006) or mice (e.g. Long, 2006; Hashimoto 2005) has been used as an animal model for schizophrenia. Phencyclidine may be administered acutely or chronically, during adulthood or during postnatal development, using different dose ranges (2.5 mg/kg to 20 mg/kg). In order to induce chronic, long lasting schizophrenic-like behaviors and neurochemical changes in the endocannabinoid system, the inventors have now developed a neonatal mouse model, based on a neonatal phencyclidine model previously described for rats (Takahashi, 2006). As the inventors have extensively studied the endocannabinoid system in Sabra strain mice (Harlan, Israel), see for example Fride 2005 and Fride 2007, Hanus 1999 these mice have been used in the present study.

The inventors have found that following administration of PCP, the CB2 receptor expression level is selectively down-regulated in different brain areas. Furthermore, up-regulation of putative complexes of CB2 receptor expression has been detected in the right cortex and basal ganglia/diencephalon of mice which were neonatally treated with phencyclidine. These results support some aspects of the teachings herein, where putative CB2 receptors complexes with a protein X (unidentified yet) are up-regulated in specific brain areas in schizophrenia while the expression of CB2 receptor monomers is down regulated.

The following conclusions have been reached from the above studies:

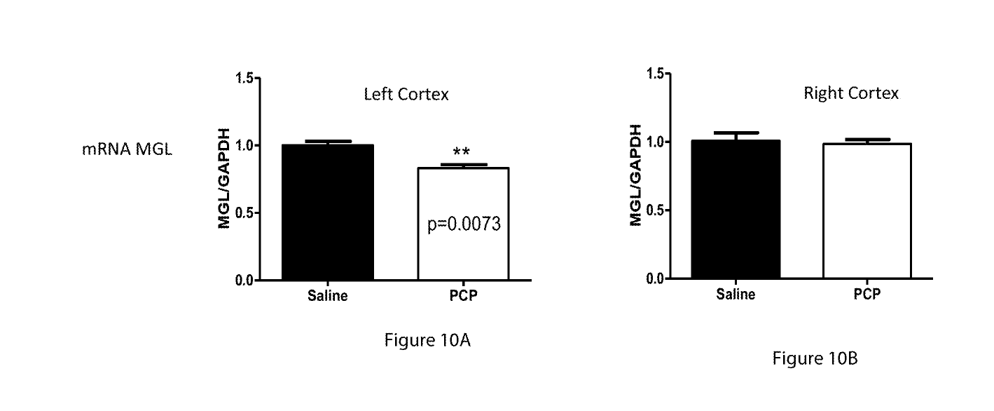

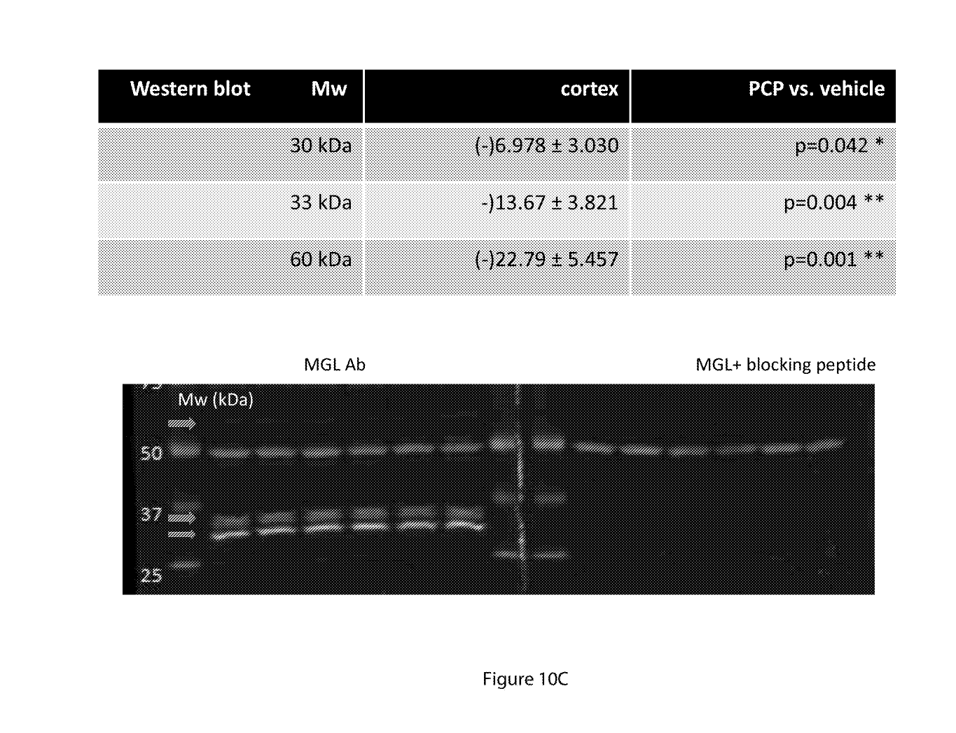

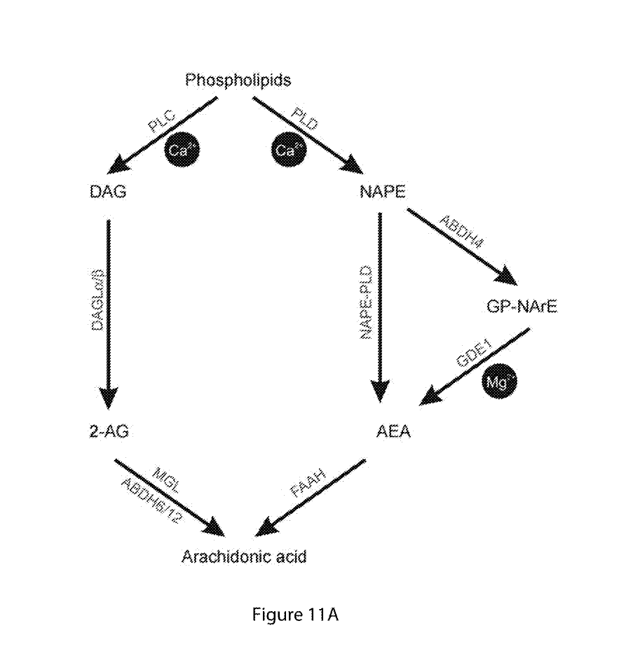

Brain tissues were analyzed for MGL, an enzyme which degrades 2-AG an endocannabinoid (Example 14, FIGS. 10A-C, FIG. 11B) in control mice and mice treated with PCP. mRNA levels of MGL decreased in the left cortex of the PCP-treated group (FIG. 10A), but not in the right cortex (FIG. 10B). The direction of these results was correlated with the reduction in protein level of MGL as analyzed with Western blotting (FIG. 10C). These results further support the combination of BCP with enzyme enhancers/inhibitors such as MGL enhancers.

A scheme of synthesis and degradative enzymes of the endocannabinoid system is shown in FIG. 11A (FIGS. 11A and 11B were published by Anavi-Goffer & Mulder, Chembiochem. 2009 10:1591-8).

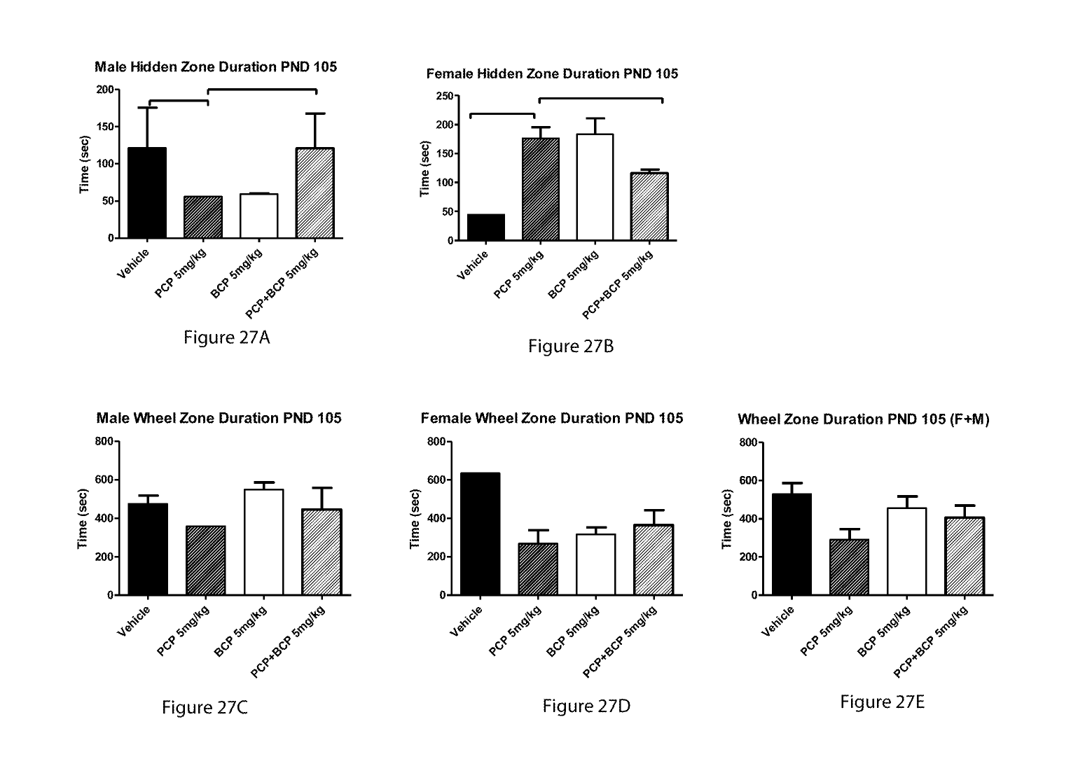

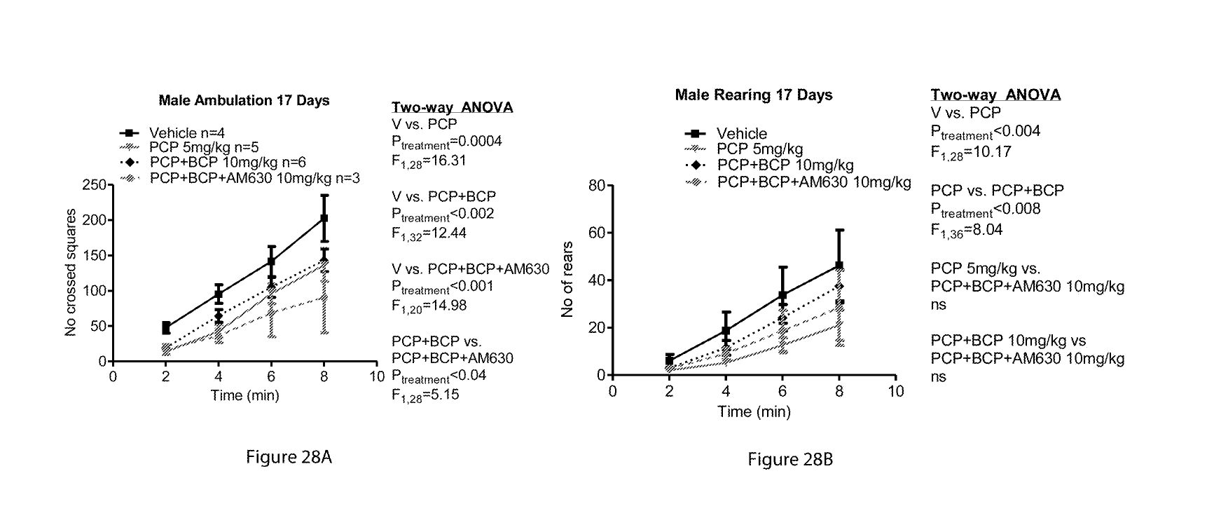

At PND 105, BCP treatment at adolescence reversed the effects of PCP on the time spend at the hidden zone (Example 15, FIGS. 27A, 27B). These results suggest that treatment with BCP reduced the level of stress and anxiety. BCP treatment at adolescence showed a trend to reverse the effect of PCP on the time spend in the wheel (FIGS. 27C, 27D, 27E).:

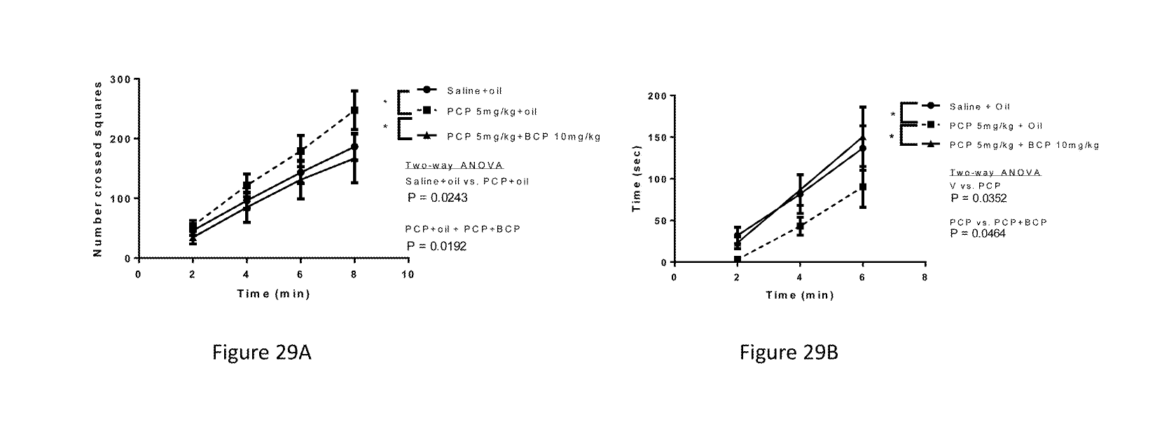

At PND 17, locomotor activity, hyperactivity, exploratory and grooming behaviors were tested with the open-field test (Example 16, FIGS. 29A and 29B). PCP significantly inhibited both ambulation and rearing behaviors. Treatment with BCP reversed the effects of PCP on rearing and exploration. AM630 reversed the effects of BCP on ambulation and rearing behaviors. The behaviors of mice that had been treated with AM630 were not significantly different from these of PCP-treated mice. AM630 is a selective CB2 receptor antagonist/inverse-agonist, therefore these results further support the CB2 receptor mediated effects of BCP.

FIG. 29A (Example 17) shows that oral treatment with 10 mg/kg BCP reversed the effect of 5 mg/kg PCP on activity of female mice in the open field test. These results show that BCP acts orally.

FIG. 29B shows that oral treatment with 10 mg/kg BCP reversed the effect of 5 mg/kg PCP on the duration of immobility of male mice in the forced swim test. These results show that BCP is orally active and reverses depression-like behavior, supporting its use as a pharmaceutical drug for the treatment of mental diseases in which depression is one of the symptoms (like for example bi-polar/mania-depressive disorder, depression, anxiety, ADHD, Tourette syndrome, depression associated with neurodegenerative diseases, depression that leads to metabolic diseases).

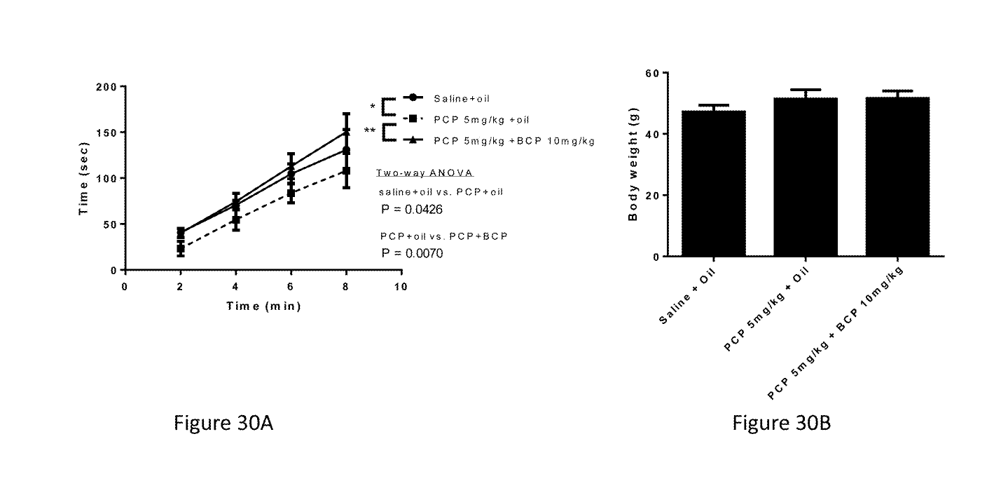

FIG. 30A shows that oral treatment with 10 mg/kg BCP reversed the effect of 5 mg/kg PCP on social interaction of male mice in the social interaction test. These results show that orally administered BCP is effective in improving social interaction, supporting its use as a drug for the treatment mental diseases in which decrease of social interaction is one of the symptoms (like for example autism, Asperger syndrome, oppositional defiant disorder, personality disorders and avoidant personality disorder).

FIG. 30B shows that oral treatment with 10 mg/kg BCP did not affect body weight of male mice at postnatal day 83. These results further support BCP as contributing to body weight control.

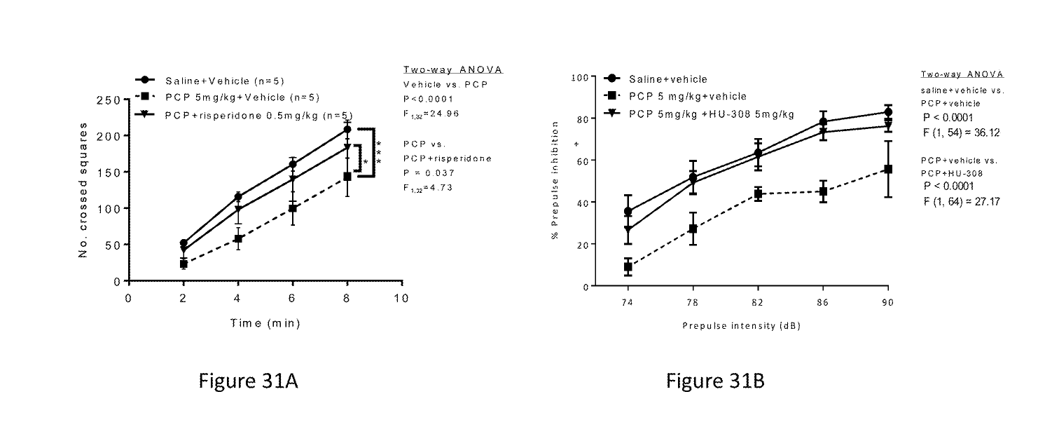

FIG. 31A (see Example 18) shows that oral treatment with 0.5 mg/kg risperidone reversed the effect of 5 mg/kg PCP on activity in the open field test of male mice at postnatal day 64. These results show that risperidone is orally active and reverses the schizophrenia-like effect of PCP in the same model that BCP was tested as mentioned above.

FIG. 31B (see Example 19) shows the effect of HU-308 in the PPI test. The prepulse inhibition test reflects the integrity of the sensor-motor gating system. Postnatal treatment with PCP reduced the percent inhibition, showing that the sensor-motor gating system is dysfunction. Postnatal treatment with HU-308 reversed the effect of PCP on the % PPI. These results show that HU-308, another CB2 receptor selective agonist, reverses schizophrenia-like behavior. As PPI reflex requires functional memory and learning processes, these results support treatment of cognitive deficits associated with mental disorders.

FIG. 34 shows that oral treatment with 5 mg/kg BCP in SEDDS formulation reversed the effect of PCP in the open field test. These results further support that:

(1) BCP is orally active;

(2) BCP in SEDDS is orally active at the same dose as the given intraperitoneal injection dose (see Example 15). These results are surprising as usually oral doses are 3 to 4 times higher than oral doses. Instead, the selected oral doses are similar to the doses given by an intraperitoneal injection;

(3) The effect of BCP in SEDDS was greater than the effect of the SEDDS vehicle on the control group, showing a synergistic effect with SEDDS formulation. Thus SEDDS formulation with antioxidants and surfactants not only stabilizes BCP but also enhances its effect. The antioxidant is part of the SEDDS vehicle.

According to an aspect of some embodiments of the teachings herein, there is provided a composition comprising beta-caryophyllene (BCP) and a pharmaceutically effective carrier for use in treating schizophrenia.

According to an aspect of some embodiments of the teachings herein, there is also provided the use of beta-caryophyllene (BCP) and a pharmaceutically effective carrier in the manufacture of a medicament for treating schizophrenia in a subject in need thereof.

In some embodiments, such a composition is formulated for administration to a human subject. In some embodiments, such a composition is formulated for administration to a non-human animal subject.

According to an aspect of some embodiments disclosed herein, there is also provided a method for treating schizophrenia in a subject in need thereof, the method comprising administering a pharmaceutically-effective amount of beta-caryophyllene (BCP) to a subject in need thereof. In some embodiments, the subject is a human subject. In some embodiments, the subject is a non-human animal.

The efficacy of the methods and compositions according to the teachings herein are demonstrated in the experimental section herein below.

According to some embodiments, the compositions and methods of treatments disclosed herein are useful for treating one or more of paranoid schizophrenia, disorganized schizophrenia, undifferentiated schizophrenia, catatonic schizophrenia, and residual schizophrenia.

In some embodiments, the compositions and methods of treatments disclosed herein are useful in the treatment of a negative symptom of schizophrenia.

In some embodiments, the compositions and methods of treatments disclosed herein are useful in the treatment of a positive symptom of schizophrenia.

In some embodiments, the compositions and methods of treatments disclosed herein are useful in the treatment of a cognitive symptom of schizophrenia.

The duration of treatment according to the method of treating schizophrenia according to the teachings is any suitable duration as determined by a treating health-care professional, typically a psychiatric doctor.

The CB2 (or specifically BCP) regimen of administration and the unit dosage administered to a mental disorder patient in need thereof depends on the age, the mode of administration, the efficiency of the composition and the mental disorder to be treated.

Thus, for example, injectable, nasal and transdermal compositions tend to need lower dosages than some oral compositions. Also, some oral compositions (like the composition detailed in Example 4) surprisingly require dosages comparable to injectable compositions such as the ones detailed in Examples 1-2).

Therefore, the dosages administered to a mental disorder patient in need thereof encompass a broad range.

In some embodiments, the CB2 receptor agonist daily dosage administered to a mental disorder patient in need thereof, by any mode of administration (including but not limited to Example 17), including but not limited to administration of slow-release/long-active formulations (including but not limited to Example 22) given on a daily basis, may vary from 0.01 mg/day to 50 mg/day (for highly selective ligands including but not limited to HU-308) or from 0.1 mg/day to 500 mg/day (for less potent agonists including but not limited to BCP) for highly effective compositions (such as oil compositions).

In some embodiments, the CB2 receptor agonist daily dosage administered to a mental disorder patient in any mode of administration, including but not limited to administration to a patient in need thereof of slow-release/long-active formulations given on a daily basis, may vary from 0.1 mg/day to 100 mg/day (for highly selective ligands including but not limited to HU-308) or from 1 mg/day to 1000 mg/day (for less potent agonists including but not limited to BCP) for less effective compositions.

Other factors determining the dosage are the age of the patient and effectiveness of the composition. In some embodiments, for HU-308 for example, a highly effective composition administered daily in any mode of administration may be given in an amount of 0.01-2 mg to infants (5-20 kg), 2-5 mg to children (20-50 kg), 5-10 mg to young adults and 10-100 mg to adults (50-100 kg). In some embodiments, for BCP for example, a highly effective composition administered daily in any mode of administration, according to some embodiments may be given in an amount of 0.1-10 mg to infants (5-20 kg), 10-20 mg to children (20-50 kg), 20-50 mg to young adults and 50-500 mg to adults (50-100 kg). These daily amounts will be administered in one or more discrete dosage units per day or, for highly effective compositions two or three times a week.

In some embodiments, the CB2 receptor agonist, for highly selective ligands including but not limited to HU-308 and for less potent agonists including but not limited to BCP, the daily dosage for less effective compositions may vary from 1 mg/day to 1000 mg/day. In some embodiments, less effective composition administered daily in any mode of administration, according to some embodiments may be given in an amount of 1-10 mg to infants (5-20 kg), 10-20 mg to children (20-50 kg), 20-50 mg to young adults and 50-500 mg to adults (50-100 kg) for highly selective ligands including but not limited to HU-308. In some embodiments, less effective composition administered daily in any mode of administration, according to some embodiments may be given in an amount of 5-20 mg to infants (5-20 kg), 20-50 mg to children (20-50 kg), 50-200 mg to young adults and 200-1000 mg to adults (50-100 kg) for less potent agonists including but not limited to BCP.

In some other embodiments of the method of treating schizophrenia according to the teachings herein, the average daily amount of the CB2 receptor selective agonist, in any mode of administration including but not limited to administration in a slow-release/long-active formulation given on a daily basis, for a human subject (especially an adult human, weighing between about 40 kg and about 120 kg) is in the range of from about (for highly potent agonists including but not limited to HU-308) 1 mg to about 25 mg from about 25 mg to about 100 mg, from about 100 mg to about 500 mg such as about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, about 130 mg, about 150 mg, about 200 mg, about 230 mg, about 350 mg, about 330 mg, about 410 mg, about 460 mg, about 520 mg, about 640 mg, about 770 mg, about 850 mg, about 930 mg, or about 1000 mg (for less potent agonists including but not limited to BCP, or for less effective compositions).

In other embodiments. the method of treating a mental disorder (or specifically schizophrenia) according to the teachings herein, the average daily amount of a CB2 receptor selective agonist or specifically BCP and/or HU-308 in any mode of administration including but not limited to administration in a slow-release/long-active formulations given on a daily basis, for a human subject (especially for an adult human, weighing between about 40 kg and about 120 kg) is in the range of from about 1 mg/day to about 5 mg/day, from about 50 mg/day to about 100 mg/day, such as about 5 mg/day, about 10 mg/day, about 20 mg/day, about 30 mg/day, about 40 mg/day, about 50 mg/day, about 60 mg/day, about 70 mg/day, about 80 mg/day, about 90 mg/day, about 100 mg/day for highly selective ligands including, but not limited to, HU-308, and is in the range of from about 10 mg/day to about 100 mg/day, such as about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, or from about 100 mg/day to about 1000 mg/day, such as about 100 mg/day, about 200 mg/day, about 300 mg/day, about 400 mg/day, about 500 mg/day, about 600 mg/day, about 700 mg/day, about 800 mg/day, about 900 mg/day or about 1000 mg/day, for less potent CB2 receptor agonists including but not limited to BCP or for less effective compositions.

In some embodiments of the method of treating schizophrenia according to the teachings herein, the average daily amount is administered with a frequency of between once per week, twice per week, 3 times per week, 4 times per week, 5 times per week, 6 times per week, once per day, twice per day, 3 times per day or 4 times per day.

In some embodiments, a composition according to the teachings herein is provided as or made as a dosage form including a plurality of discrete units (e.g., discrete solids or metered liquids, sprays), especially discrete solid units such as pills (including tablets and caplets) and capsules (including gelcaps), wherein each unit includes a CB2 receptor selective agonist or specifically BCP or HU-308 in the range of from about 0.05 mg to about 1000 mg, from about 0.1 mg to about 1000 mg, from about 1 mg to about 1000 mg, from about 10 mg to about 1000 mg, from about 100 mg to about 1000 mg, for example about 0.05 mg, about 0.1 mg, about 0.5 mg, about 1 mg, about 5 mg, about 10 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 200 mg, about 300 mg, about 400 mg, about 500 mg for highly selective ligands including, but not limited to HU-308, and in the range of from about 0.1 mg, about 0.5 mg, about 1 mg, about 5 mg, about 10 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 200 mg, about 300 mg, about 400 mg, about 500, about 600 mg, about 700 mg, about 800 mg, about 900 mg, or about 1000 mg for less potent CB2 receptor agonists including but not limited to BCP or for less effective compositions. In some such embodiments, such a dosage form is useful for the once-daily administration of the desired average daily dosage, according to the age of the patient.

In some embodiments, the dosage of the CB2 receptor selective agonist or specifically BCP and/or HU-308 administered to a mental disorder patient for highly effective sustained-release delivery compositions (such as compositions for a slow-release, slow-acting form of medication prepared as a capsule or as a depot injection given for example but not limited to intramuscular injection, administered once a week or once a month to up to every six months) may vary from 100 mg/single administration (for highly potent agonists including but not limited to HU-308 or for weekly injection) to 3000 mg/single administration (for less potent agonists including but not limited to BCP or for injection every 3 months).

Sustained release dosage forms are designed to release a drug at a predetermined rate in order to maintain a constant drug concentration for a specific period of time with minimum side effects. This can be achieved through a variety of formulations, including liposomes and drug-polymer conjugates (an example being hydrogels).

Depot injection is the administration of a sustained-action drug composition that allows slow release and gradual absorption, so that the active agent can act for much longer periods than is possible with standard injections. Depot injections are usually administered deep intramuscularly.

The factors determining the dosage are the age of the patient, the effectiveness of the composition and the CB2 receptor selective agonist used. In some embodiments, for HU-308 for example, a sustained-release delivery composition administrated by injection should be given at 0.5-10 mg to infants (5-20 kg), 10-20 mg to children (20-50 kg) and from 20-100 mg to 100-1000 mg to adults (50-100 kg). In some embodiments, for BCP for example, a sustained-release delivery composition administered by injection may be given at 1-50 mg to infants (5-20 kg), 50-100 mg to children (20-50 kg) and from 100-200 mg to 200-3000 mg to adults (50-100 kg).

In some embodiments, the CB2 receptor selective agonist or specifically BCP and/or HU-308 in dosage for sustained-release delivery compositions (such as compositions for a slow-release, slow-acting form of medication prepared as a capsule or a depot injection given for example but not limited by intramuscular injection, which are administrated every 1 week, once a month and to up to every three months) may vary from 0.1 mg/single administration to 250 mg/single administration for highly potent agonists including but not limited to HU-308 and from 1 mg/single administration to 500 mg/single administration for less potent agonists including but not limited to BCP.

In some embodiments, the CB2 receptor selective agonist or specifically BCP and/or HU-308 in dosage for sustained release delivery compositions (such as compositions for a slow-release, slow-acting form of medication prepared as a capsule or a depot injection given for example but not limited by intramuscular injection, which are administrated once a month and up to every six months) may vary from 0.5 mg/single administration to 1000 mg/single administration (for highly potent agonists including but not limited to HU-308) and from 1 mg/single administration to 3000 mg/single administration (for less potent agonists including but not limited to BCP).

Another factor determining the dosage is the effectiveness of the composition. In some embodiments, the dosage for less effective long term delivery compositions in all modes of administration may vary from 1 mg/day to 3000 mg/day. In some embodiments, the CB2 receptor agonists dosage for sustained-release delivery compositions (such as compositions for a slow-release, slow-acting form of medication prepared as a capsule or a depot injection given for example but not limited by intramuscular injection) may vary from 1 mg/single administration to 1000 mg/single administration (for highly potent CB2 receptor agonists including, but not limited to, HU-308) or from 10 mg/single administration to 3000 mg/single administration (for less potent CB2 receptor agonists including, but not limited to, BCP).

Another factor determining the dosage is the age of the patient. In some embodiments, for HU-308 for example, a sustained-release delivery composition for a slow-release, slow-acting form of medication prepared as a capsule or a depot injection given for example but not limited by intramuscular injection, which are administrated once a week, once a month and to up to once every six months) according to some embodiments may be given at an amount of 0.1-10 mg to infants (5-20 kg), 5-20 mg to children (20-50 kg) and from 10-100 mg to 50-1000 mg to adults (50-100 kg). In some embodiments, for BCP for example, a sustained-release delivery composition for a slow-release, slow-acting form of medication prepared as a capsule or as a depot injection given for example but not limited to intramuscular injection, which are administrated every 1 week, once a month and to up to every six months, according to some embodiments may be given at an amount of 1-50 mg to infants (5-20 kg), 20-100 mg to children (20-50 kg), 50-200 mg to young adults and from 100-3000 mg to adults (50-100 kg).

In some embodiments, the administration regimen of a CB2 receptor selective agonist or specifically BCP and/or HU-308 in sustained-release delivery composition is one administration per week, to once every two weeks, to one administration per a month, to one administration per each other month or once every six months as required.

In some other embodiments of the method of treating schizophrenia according to the teachings herein, the average amount (in mg) per a single administration of a sustained-release delivery composition for, mainly by injection, (once a week and up to every six months) for a human subject (especially an adult human, weighing between about 40 kg and about 120 kg)

is in the range of from about (for highly potent CB2 receptor agonists including, but not limited to, HU-308) 10 mg to about 25 mg, from about 25 mg to about 100 mg, from about 100 mg to about 500 mg, such as about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, about 130 mg, about 150 mg, about 200 mg, about 230 mg, about 350 mg, about 330 mg, about 410 mg, about 460 mg, about 500 mg, from about 500 mg to about 1000 mg, such as about 650 mg, about 730 mg, about 840 mg, about 960 mg, about 1000 mg, from about 1000 mg to about 3000 mg, such as about 1200 mg, about 1800 mg, about 2300 mg, about 2500 mg or about 3000 mg (for less potent CB2 receptor agonists including, but not limited to, BCP or for less effective compositions).

In other embodiments of the method of treating a mental disorder (or specifically schizophrenia) with a CB2 receptor selective agonist or specifically BCP and/or HU-308 according to the teachings herein, the average amount (in mg) per a single administration of a sustained-release delivery composition mainly by injection (once a week and up to every six months) for a human subject (especially an adult human, weighing between about 40 kg and about 120 kg) is in the range of from about 10 mg/single administration to about 50 mg/single administration from about 50 mg/single administration to about 100 mg/single administration, such as about 20 mg/single administration, about 30 mg/single administration, about 60 mg/single administration from about 100 mg/single administration to about 1000 mg/single administration, such as about 200 mg/single administration, about 300 mg/single administration, about 400 mg/single administration, about 500 mg/single administration, about 600 mg/single administration, about 700 mg/single administration, about 800 mg/single administration, about 900 mg/single administration, from about 1000 mg/single administration (for highly potent CB2 receptor agonists including, but not limited to, HU-308) and is in the range of from about 100 mg/single administration to about 3000 mg/single administration, such as about 200 mg/single administration, about 300 mg/single administration, about 400 mg/single administration, about 500 mg/single administration, about 600 mg/single administration, about 700 mg/single administration, about 800 mg/single administration, about 900 mg/single administration, from about 1000 mg/single administration to about 3000 mg/single administration, such as about 1250 mg/single administration, about 1600 mg/single administration, about 2100 mg/single administration, about 2400 mg/single administration, about 2700 mg/single administration, or about 3000 mg/single administration (for less potent CB2 receptor agonists including, but not limited to, BCP) or for less effective compositions.

In some embodiments of the method of treating schizophrenia according to the teachings herein, the average amount of a single administration mainly, but not limited to injection or oral administration is administered with a frequency of between about once a month to once every two months, to about once every three months, to about once every four months, to about once every five months, to about once every six months. In some embodiments, a composition according to the teachings herein is provided as or made as a dosage form including a plurality of discrete units (e.g., discrete solids or metered liquids, sprays, depot formulation for injection), especially discrete solid units such as pills (including tablets and caplets) and capsules (including gelcaps), where each unit includes a CB2 receptor selective agonist or specifically BCP and HU-308) in the range of from about 10 mg to about 1000 mg, such as about 10 mg, such as about 50 mg, such as about 100 mg, such as about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg for highly selective ligands including, but not limited to, HU-308, and in the range of from about 100 mg to about 3000 mg, such as about 10 mg, such as about 50 mg, such as about 100 mg, such as about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1500 mg, about 2000 mg, about 2500 mg, or about 3000 mg for less potent CB2 receptor agonists including, but not limited to BCP, or for less effective compositions. In some such embodiments, such a dosage form is useful for a single administration of the desired average dosage per single administration.

According to some embodiments, the compositions of this invention may be administered by any suitable route of administration, including but not limited to oral, parenteral, topical, intranasal, vaginal, inhalation, transdermal or rectal administration.

According to an embodiment, there is provided an oral composition formulated as a tablet, sublingual tablet, caplet, depot, transdermal gel, cream, topical spray, nasal spray, transdermal patch, spray, suppository, chewable, capsule, dragee, powder, granules, suspension, solution, emulsion, syrup, transmucosal, lozenge, sachet, gastroresistant oral dosage, gastroresistant softgel capsule, sprinkle and an ingestible solution.

The parenteral composition is formulated as an injectable solution, injectable suspension or injectable depot and is administered as intravenous injection, intramuscular injection, intradermal injection, intraperitoneal injection, intrathecal injection or subcutaneous injection, intra-arterial injection and injectable suspension, according to case. The topical composition is formulated as a transdermal gel, cream, patch or topical spray. The intranasal composition is formulated as a nasal spray.

In an embodiment, the composition is a gastroresistant oral dosage form, that is to say, an orally-administrable dosage form configured to carry the active(s) through the stomach to be released into contact with the digestive tract only after passage through the duodenum. As an example, in some such embodiments, the composition is in the form of a gastroresistant soft gel capsule, comprising between 5 mg and about 1000 mg BCP and/or 0.5 mg to 500 mg HU-308 in a carrier. Some embodiments of the method, when implemented with an adult human subject, comprise orally ingesting a single capsule twice a day for at least one a month or once every two months, to about once every three months, to about once every four months, to about once every five months, to about once every six months, so that the average daily dose is between about 5 mg and about 1000 mg BCP.

In some embodiments, the composition described herein further comprises at least one antipsychotic agent, such as, for example, a typical antipsychotic agent including, but not limited to, one or more of chlorpromazine, haloperidol, perphenazine, pimozide or fluphenazine, and/or an atypical antipsychotic agent including, but not limited to, one or more of clozapine, risperidone, olanzapine, quetiapine, ziprasidone, aripiprazole, sertindole, amisulpride, paliperidone, paliperidone palmitate, and combinations thereof.

In some embodiments of the method of treatment, the CB2 receptor selective agonist or for example BCP is administered together with at least one antipsychotic agent, such as, for example, a typical antipsychotic agent including, but not limited to, one or more of benperidol, bromperidol, droperidol, haloperidol, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, amisulpride, amoxapine, aripiprazole, dehydroaripiprazole, asenapine, cariprazine, clozapine, blonanserin, iloperidone, lurasidone, melperone, nemonapride, olanzapine, paliperidone, paliperidone palmitate, perospirone, quetiapine, remoxipride, risperidone, sertindole, sultopride, trimipramine, ziprasidone, brexpiprazole, ITI-007, pimavanserin, RP5063 (RP5000) cannabidiol (CBD), cannabidivarin (CBDV), cannabiodiolic acid (CBDA), tetrahydrocannabivarin (THCV), OPC-14857, DM-1458, DM-1451, DM-1452, DM-1454, DCPP, cannabigerol (CBG) and its analogs CBGA and CBGV and combinations thereof.

In some embodiments where the CB2 receptor selective agonist or for example BCP and an antipsychotic agent are administered together, the two active agents are co-administered in a single dosage form.

In some embodiments where the CB2 receptor selective agonist or for example BCP and antipsychotic agents are administered together, the two or more active agents are co-administered in a single dosage form.

In some embodiments where the BCP and an antipsychotic agent are administered together, the CB2 receptor selective agonist or for example BCP and the antipsychotic agent/s are co-administered in separate dosage forms, either sequentially or simultaneously. For example, the additional antipsychotic agent/s may be administered prior to administration of the CB2, or the additional antipsychotic agent may be administered subsequent to administration of CB2.

While not wishing to be bound to any one theory, the inventors consider that it is likely that at least part, if not all, of the herein demonstrated efficacy of the CB2 in general or BCP in particular in treating schizophrenia relates to the CB2 receptor selective agonist properties.

Thus, according to an aspect of some embodiments of the teachings herein, there is provided a composition comprising a CB2 receptor selective agonist and a pharmaceutically effective carrier for use in treating schizophrenia.

According to an aspect of some embodiments of the teachings herein, there is also provided the use of a CB2 receptor selective or highly selective agonist and a pharmaceutically effective carrier in the manufacture of a medicament for treating schizophrenia in a subject in need thereof.

According to an aspect of some embodiments of the teachings herein, there is also provided a method for treating schizophrenia in a subject in need thereof, the method comprising administering a pharmaceutically-effective amount of a CB2 selective receptor agonist to the subject.

In an embodiment, there is provided a stable composition, said composition comprising at least one selective Cannabinoid Receptor Type 2 (“CB2”) receptor agonist wherein selected from the group consisting of HU-433, HU-910, HU-914, CB 65, GP 1a, GP 2a, GW 405833, JWH 015, JWH 133, AM1241, L-759,656, L-759,633, MDA 19, SER 601, BML-190, N-alkylamide, rutamarin, diindolylmethane (DIM), cannabilactones and a pharmaceutically effective carrier; wherein the composition is used to treat a patient suffering from a mental disorder, and wherein the at least one selective CB2 receptor agonist is in an amount sufficient to treat the patient suffering from a mental disorder.

According to an embodiment, there is provided a stable composition, wherein said composition comprises at least one selective Cannabinoid Receptor Type 2 (“CB2”) receptor agonist, wherein selected from the group consisting of HU-433, HU-910, HU-914, CB 65, GP 1a, GP 2a, GW 405833, JWH 015, JWH 133, AM1241, L-759,656, L-759,633, MDA 19, SER 601, BML-190, N-alkylamide, rutamarin, diindolylmethane (DIM), cannabilactones and combinations thereof; at least one additional active agent selected from the group consisting of at least one antipsychotic agent, at least one GPR55 modulator, at least one terpene/terpenoid, at least one anti-inflammatory agent, at least one enzyme enhancer, at least one enzyme inhibitor, at least one antidepressant, at least one anxiolytic, at least one cognitive enhancer, at least one anti-diabetic agent; and at least one pharmaceutically effective carrier; wherein said composition is used to treat a patient suffering from a mental disorder, wherein the at least one selective CB2 receptor agonist is in an amount sufficient to treat the patient suffering from a mental disorder, and wherein the at least one additional active agent is in an amount sufficient to treat the patient suffering from a mental disorder.

According to an embodiment, there is provided a stable composition, wherein said composition comprises at least one selective Cannabinoid Receptor Type 2 (“CB2”) receptor agonist and at least one selective enzyme inhibitor and at least one enzyme enhancer selected from the group targeting the enzymes cyclooxygenase-2 (COX-2), fatty acid amide hydrolase (FAAH), monoacylglycerol lipase (MGL), α/β-hydrolase domain containing 6 (ABDH6 or ABHD6), α/β-hydrolase domain containing 12 (ABDH12), α/β-hydrolase domain containing 4 (ABDH4), sn-1-diacylglycerol lipase alpha (DAGLalpha), sn-1-diacylglycerol lipase beta (DAGLbeta), N-acyl phosphatidylethanolamine phospholipase D (NAPE-PLD), phosphodiesterase 1 (GDE1), phospholipase C (PLC), phospholipase D (PLD) and combination thereof; and a pharmaceutically effective carrier, wherein the composition is used to treat a patient suffering from a mental disorder, and wherein the at least one additional active agent is in an amount sufficient to treat the patient suffering from a mental disorder.

According to another embodiment, there is provided a stable composition, said composition comprising at least one selective Cannabinoid Receptor Type 2 (“CB2”) receptor agonist, wherein is selected from the group consisting of beta-caryophyllene (“BCP”), [(1R,2R,5R)-2-[2,6-dimethoxy-4-(2-methyloctan-2-yl)phenyl]-7,7-dimethyl-4-bicyclo[3.1.1]hept-3-enyl]methanol (“HU-308”) and combinations thereof; and a pharmaceutically effective carrier, wherein the composition is used to treat a patient suffering from a mental disorder other than schizophrenia, and wherein the at least one selective CB2 receptor agonist is in an amount sufficient to treat the patient suffering from a mental disorder other than schizophrenia.

In an embodiment, there is provided a stable composition, wherein said composition comprises at least one selective Cannabinoid Receptor Type 2 (“CB2”) receptor agonist, wherein said at least one selective CB2 receptor agonist is selected from the group consisting of beta-caryophyllene (“BCP”), [(1R,2R,5R)-2-[2,6-dimethoxy-4-(2-methyloctan-2-yl)phenyl]-7,7-dimethyl-4-bicyclo[3.1.1]hept-3-enyl]methanol (“HU-308”) and combinations thereof; the at least one additional active agent is selected from the group consisting of at least one antipsychotic agent, at least one GPR55 modulator, at least one terpene/terpenoid, at least one anti-inflammatory agent, at least one enzyme enhancer, at least one enzyme inhibitor, at least one antidepressant, at least one anxiolytic, at least one cognitive enhancer, at least one anti-diabetic agent and combinations thereof; and at least one pharmaceutically effective carrier, wherein said composition is used to treat a patient suffering from a mental disorder other than schizophrenia, wherein the at least one selective CB2 receptor agonist is in an amount sufficient to treat a patient suffering from a mental disorder other than schizophrenia, and wherein the at least one additional active agent is in an amount sufficient to treat the patient suffering from a mental disorder other than schizophrenia.

According to another embodiment, there is provided a stable composition, said composition comprising at least one selective Cannabinoid Receptor Type 2 (“CB2”) receptor agonist wherein the CB2 receptor selective agonist is selected from the group consisting of beta-caryophyllene (“BCP”), [(1R,2R,5R)-2-[2,6-dimethoxy-4-(2-methyloctan-2-yl)phenyl]-7,7-dimethyl-4-bicyclo[3.1.1]hept-3-enyl]methanol (“HU-308”), at least one antioxidant and combinations thereof; and a pharmaceutically effective carrier, wherein said composition is used to treat a patient suffering from a mental disorder and wherein the at least one selective CB2 receptor agonist is in an amount sufficient to treat the patient suffering from a mental disorder.

In an embodiment, there is provided a stable composition, said composition comprising at least one selective Cannabinoid Receptor Type 2 (“CB2”) receptor agonist, wherein the at least one selective CB2 receptor agonist is selected from the group consisting of beta-caryophyllene (“BCP”), [(1R,2R,5R)-2-[2,6-dimethoxy-4-(2-methyloctan-2-yl)phenyl]-7,7-dimethyl-4-bicyclo[3.1.1]hept-3-enyl]methanol (“HU-308”) and combinations thereof and at least one additional active agent selected from the group consisting of at least one antipsychotic agent, at least one GPR55 modulator, at least one terpene/terpenoid, at least one anti-inflammatory agent, at least one enzyme enhancer, at least on enzyme inhibitor, at least one antidepressant, at least one anxiolytic, at least one cognitive enhancer, at least one anti-diabetic agent and combinations thereof; and at least one pharmaceutically effective carrier; wherein said composition is used to treat a patient suffering from a mental disorder and wherein the at least one selective CB2 receptor agonist is in an amount sufficient to treat a patient suffering from a mental disorder and wherein the at least one additional active agent is in an amount sufficient to treat the patient suffering from a mental disorder.

In another embodiment, there are provided the above compositions, in which the at least one additional active agent is selected from the group consisting of haloperidol, chlorpromazine, fluphenazine, perphenazine, aripiprazole, clozapine, olanzapine, paliperidone, paliperidone palmitate, quetiapine, risperidone, ziprasidone, benperidol, bromperidol, droperidol, timiperone, fluspirilene, penfluridol, pimozide, acepromazine, cyamemazine, dixyrazine, levomepromazine, mesoridazine, perazine, pericyazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, amisulpride, amoxapine, dehydroaripiprazole, asenapine, cariprazine, blonanserin, iloperidone, lurasidone, melperone, nemonapride, perospirone, remoxipride, sertindole, sultopride, trimipramine, brexpiprazole, ITI-007, pimavanserin, RP5063 (RP5000), cannabidiol (CBD), cannabidivarin (CBDV), cannabiodiolic acid (CBDA), tetrahydrocannabivarin (THCV), OPC-14857, DM-1458, DM-1451, DM-1452, DM-1454, DCPP, cannabigerol (CBG), CBGA, CBGV their analogs and derivatives and combinations thereof.

The compositions of this invention may be stabilized and the therapeutic activity of a selective Cannabinoid Receptor Type 2 (“CB2”) receptor agonist may be enhanced by addition of an antioxidant or a free-radical scavenger, which can be selected from vitamin E, tocopherols, tocopherol esters, vitamin C, beta-carotene, butylated hydroxy toluene, butylated hydroxyanisole or other FDA-approved antioxidant listed in the FDA's Inactive Ingredients Database (IID). The antioxidant is not DMSO or ethanol.

In an embodiment, the compositions of this invention are formulated for oral, parenteral, topical, intranasal, vaginal, inhalation, transdermal or rectal administration.

In another embodiment, the composition of this invention, are formulated as a tablet, sublingual tablet, caplet, depot, transdermal gel, cream, topical spray, nasal spray, transdermal patch, spray, suppository, chewable, capsule, dragee, powder, granules, suspension, solution, emulsion, syrup, transmucosal, lozenge, sachet, gastroresistant oral dosage, gastroresistant softgel capsule, sprinkle or an ingestible solution.

Alternatively, the compositions of this invention are formulated as an injectable solution and administered as intravenous injection, intra-arterial injection, intramuscular injection, intradermal injection, intraperitoneal injection, intrathecal injection, depot injection, subcutaneous injection or injectable suspension, according to case.

According to an embodiment, there are provided compositions of this invention wherein the at least one CB2 receptor selective agonist is BCP and the at least one additional active agent is selected from the group consisting of risperidone, paliperidone, paliperidone palmitate, aripiprazole, quetiapine, CBD and derivatives and analogs, CBG and derivatives and analogs, THCV and derivatives and analogs, brexpiprazole and combinations thereof.

In an embodiment, the parenteral compositions of this invention are formulated as an injectable solution and administered as intravenous injection, intra-arterial injection, intramuscular injection, intradermal injection, intraperitoneal injection, intrathecal injection, depot injection, subcutaneous injection or injectable suspension, according to case.

In another embodiment, the composition of this invention are formulated as a tablet, sublingual tablet, caplet, depot, transdermal gel, cream, topical spray, nasal spray, transdermal patch, spray, suppository, chewable, capsule, dragee, powder, granules, suspension, solution, emulsion, syrup, transmucosal, lozenge, sachet, gastroresistant oral dosage, gastroresistant softgel capsule, sprinkle or an ingestible solution.

According to an embodiment, the composition of this invention is formulated in a carrier comprising dimethyl sulfoxide (DMSO) ethanol or their mixtures.

In an embodiment, there are provided compositions in which the at least one CB2 receptor selective agonist is beta caryophyllene (BCP) and the at least one additional active agent is an antipsychotic agent selected from the group consisting of risperidone, paliperidone, paliperidone palmitate, aripiprazole, quetiapine, CBD and derivatives and analogs, CBG and derivatives and analogs, THCV and derivatives and analogs, brexpiprazole and combinations thereof.

According to another embodiment, the compositions of this invention comprise either one of the two BCP isomers E-BCP and Z-BCP wherein in substantially pure form and substantially free of α-humulene, BCP oxide, copaene, eugenol, δ-cadinene, or mixtures thereof.

According to another embodiment, the compositions of this invention comprise either one of the two BCP isomers E-BCP and Z-BCP as a selective Cannabinoid Receptor Type 2 (“CB2”) receptor agonist and in addition various amounts of alpha-humulene, copaene, eugenol, δ-cadinene, BCP oxide; and mixtures and combinations thereof.

Alternatively, said compositions, comprise at least 98% w/w substantially pure isomer E-BCP, wherein substantially free of BCP oxide and α-humulene.

In another alternative, the compositions of this invention comprise at least 98% w/w substantially pure isomer Z-BCP wherein substantially free of BCP oxide and α-humulene.

Alternatively, the compositions of this invention comprise at least 85% w/w substantially pure isomer either E-BCP or Z-BCP and about 1% to 15% α-humulene.

In a further alternative, the compositions of this invention comprise at least 85% w/w substantially pure isomer E-BCP and about 13% alpha-humulene, about 1% copaene, about 0.3% eugenol, about 0.3% δ-cadinene and about 0.3% BCP oxide.

In an embodiment, the compositions of this invention comprise at least 85% w/w substantially pure isomer Z-BCP and about 13% alpha-humulene, about 1% copaene, about 0.3% eugenol, about 0.3% δ-cadinene and about 0.3% BCP oxide.

According to an embodiment, there is provided a method of treatment of a mental disorder in a patient in need thereof, said method comprising administering the compositions of this invention, in an amount sufficient to treat a patient suffering from a mental disorder.

According to another embodiment, there is provided a method of treatment of a mental disorder other than schizophrenia, wherein the method comprises administering the composition comprising at least one selective Cannabinoid Receptor Type 2 (“CB2”) receptor agonist, wherein is selected from the group consisting of beta-caryophyllene (“BCP”), [(1R,2R,5R)-2-[2,6-dimethoxy-4-(2-methyloctan-2-yl)phenyl]-7,7-dimethyl-4-bicyclo[3.1.1]hept-3-enyl]methanol (“HU-308”) and combinations thereof, in an amount sufficient to treat a patient suffering from a mental disorder other than schizophrenia.

According to another embodiment, there is provided a method of treatment of a mental disorder other than schizophrenia, wherein the method comprises administering the composition comprising at least one selective Cannabinoid Receptor Type 2 (“CB2”) receptor agonist, wherein said at least one selective CB2 receptor agonist is selected from the group consisting of beta-caryophyllene (“BCP”), [(1R,2R,5R)-2-[2,6-dimethoxy-4-(2-methyloctan-2-yl)phenyl]-7,7-dimethyl-4-bicyclo[3.1.1]hept-3-enyl]methanol (“HU-308”) and combinations thereof; the at least one additional active agent is selected from the group consisting of at least one antipsychotic agent, at least one GPR55 modulator, at least one terpene/terpenoid, at least one anti-inflammatory agent, at least one enzyme enhancer, at least one enzyme inhibitor, at least one antidepressant, at least one anxiolytic, at least one cognitive enhancer, at least one anti-diabetic agent, and combinations thereof; and at least one pharmaceutically effective carrier wherein the at least one selective CB2 receptor agonist is in an amount sufficient to treat a patient suffering from a mental disorder other than schizophrenia, and wherein the at least one additional active agent is in an amount sufficient to treat the patient suffering from a mental disorder other than schizophrenia.

According to an embodiment, there is provided a method of treatment of a mental disorder in a patient in need thereof by treatment with a composition of this invention, wherein the mental disorder is selected from the group consisting of schizophrenia, bipolar disorder I and II, unipolar disorder, multiple personality disorder, psychotic disorders, depression, psychotic depression, depressive disorders, major depressive disorder, stereotypic movement disorder, autism spectrum disorders, obsessive-compulsive disorder (OCD), bacterial-induced tic disorder, pediatric autoimmune neuropsychiatric disorders associated with streptococcal infections (PANDAS), chorea (Sydenham's chorea (SC), chorea minor, chorea gravidarum, drug-induced chorea), drug-induced repetitive behaviors, akathisia, dyskinesias, Wernicke-Korsakoff syndrome, Tourette's syndrome, tic disorders, epilepsy, anxiety disorders, autistic spectrum disorder, enuresis, addiction, withdrawal symptoms associated with addiction, Asperger syndrome, oppositional defiant disorder, behavioral disturbance, agitation, psychosis/agitation associated with Alzheimer's disease, psychosis associated with Parkinson's disease, psychosis associated with drug of abuse, psychosis associated with psychedelic drug abuse, LSD-induced psychosis, steroid-induced schizophrenia, steroid-induced psychosis, Capgras syndrome; Fregoli syndrome; Cotard syndrome, personality disorders, borderline personality disorder, avoidant personality disorder, attention-deficit/hyperactive disorder (ADHD, ADD, HD), mania, dementia, anorexia, anorexia nervosa, anxiety, generalized anxiety disorder, social anxiety disorder, body dismographic disorder, obsessive compulsive disorder, paranoid disorder, nightmares, agitation, post-traumatic stress disorder (PTSD), severe mood dysregulation, mental disorder such as depression or anxiety that leads to metabolic diseases such as obesity, depression associated with any of the above clinical conditions and cognitive deficits associated with any of the above clinical conditions.

In an embodiment, there is provided a method of treatment of a mental disorder in a patient in need thereof by treatment with a composition of this invention, wherein said mental disorder is schizophrenia and wherein said schizophrenia includes any symptom and its onset is at any age.

The above method of treatment, wherein the mental disorder is schizophrenia of all types, the CB2 receptor selective agonist is BCP and the at least one additional active agent is selected from the group consisting of risperidone, paliperidone, paliperidone palmitate, aripiprazole, quetiapine, CBD and derivatives and analogs, CBG and derivatives and analogs, THCV and derivatives and analogs, brexpiprazole and combinations thereof.

In an embodiment, there is provided a method of treatment of a mental disorder in a patient in need thereof, wherein said method comprises administering the composition of this invention in an amount sufficient to treat a patient suffering from a mental disorder, wherein the at least one CB2 receptor selective agonist in substantially pure form is beta caryophyllene (E-BCP and/or Z-BCP) or HU-308 as sole active agent and the mental disorder is bi-polar disorder, onset at any age.

In another embodiment, there is provided a method of treatment a mental disorder in a patient in need thereof, wherein said method comprises administering a composition of this invention, wherein the at least one CB2 selective receptor agonist is beta caryophyllene (E-BCP and/or Z-BCP) or HU-308 as sole active agent and the mental disorder is selected from the group consisting of psychosis associated with psychedelic drug abuse and LSD-induced psychosis, onset at any age.

The administration of the psychedelic drug DOI ((−)-1-(2,5-Dimethoxy-4-iodophenyl)-2-aminopropane hydrochloride also named (−)-2,5-dimethoxy-4-iodoamphetamine hydrochloride) induces behavioral symptoms in a murine model. DOI-induced psychotic like behavior is blocked by antipsychotic drugs like pimozide. The present invention discloses that administration of DOI to mice at postnatal age of three to six weeks increased grooming response.

In some embodiments, the DOI model (see Example 20) supports the activity of selective CB2 receptor agonist as an antipsychotic against LSD-induced psychosis.

According to some embodiments, the present invention discloses the unexpected findings that exposure to HU-308, (a selective CB2 receptor agonist) reverses the effects of the psychedelic drug DOI on behavioral symptoms in a murine model. Noteworthy, HU-308 inhibits DOI-induced psychotic-like behaviour already after about one hour. This time line suggests that CB2 receptors in the periphery are not involved in the inhibition of DOI-induced psychotic-like behaviour. This is in contrast to the recitals of Darmani et al, 2001 stating that non-selective mixed CB1/CB2 receptor agonists are able to reverse the DOI-induced psychotic like behavior (see Darmani, 2001 in References below). Based on the above findings, the present invention discloses the activity of HU-308 as an antipsychotic against LSD induced psychosis.

There is provided the use of a therapeutically effective dose of at least one CB2 receptor selective agonist in substantially pure form in a pharmaceutically effective carrier and optionally of a therapeutically effective dose of at least one antipsychotic agent, at least one GPR55 modulator, at least one anti-inflammatory agent, at least one enzyme enhancer, at least on enzyme inhibitor, at least one anti-depressant, at least one anxiolytic, at least one cognitive enhancer, at least one anti-diabetic agent, in the manufacture of a composition for treating a mental disorder in a subject in need thereof.

According to an embodiment, there is provided a method of treatment of a mental disease in a patient in need thereof with a composition of this invention, wherein said composition is administered to a patient in need thereof from about once a month to about once every two months, to about once every three months, to about once every four months, to about once every five months, to about once every six months, to about once per week, twice per week, 3 times per week, 4 times per week, 5 times per week, 6 times per week, once per day, twice per day or 3 times per day.

According to another embodiment there is provided a method of treatment, wherein a composition of this invention is administered once a week to about 3 times per day, once per week, twice per week, 3 times per week, 4 times per week, 5 times per week, 6 times per week, once per day, twice per day or 3 times per day to a patient suffering from a mental disorder in need thereof.

In some embodiments, there is provided a method of treatment of a mental disorder, specifically schizophrenia in a patient in need thereof, by administration of a composition of this invention, wherein the average daily amount of said either BCP, HU-308 administered in any daily mode of administration, including but not limited to administration in sustained-release compositions given on a daily basis, is in a range selected from the group consisting of 0.01-0.1 mg, 0.1-1 mg 1-10 mg, 10-25 mg, 25-100 mg, 100-1000 mg, according to the age and the effectiveness of the composition.

Sustained release compositions are pharmaceutical compositions that do not immediately disintegrate and release the active ingredient(s) into the body. An example is enteric coated oral medications, which dissolve in the intestines rather than the stomach. Other sustained release compositions are formulated by various formulations including coatings, polymers, encapsulation, etc.

In some embodiments, there is provided a method of treatment of a mental disorder, specifically schizophrenia, by administration of a composition of this invention, wherein the average amount of a single administration of a sustained-release delivery composition is selected from compositions for slow-release, sustained-release drugs formulated as a capsule or as a depot injection given either orally or mostly by injection, administrated once a week or once a month to up to every six months comprising BCP, HU-308 administered in amount selected from 0.1-10 mg, 10-25 mg, 25-100 mg, 100-1000 mg or 100-3000 mg, according to patient's age and composition's effectiveness.

In the methods of treatment of this invention, said at least one additional active agent in any of the compositions of this invention is co-administered in a single dosage form together with said at least one CB2 receptor selective agonist.

Alternatively, in the methods of treatment of this invention, said at least one additional active agent in any of the compositions of the present disclosure is co-administered sequentially in a dosage form separate from said CB2 receptor selective agonist wherein in either order.

Exemplary embodiments of the teachings herein are discussed herein below with reference to specific materials, methods and examples. The material, methods and examples discussed herein are illustrative and not intended to be limiting. In some embodiments, methods and materials similar or equivalent to those described herein are used in the practice or testing of embodiments of the invention. It is to be understood that the invention is not necessarily limited in its application to the details of construction and the arrangement of the components and/or methods set forth in the following description and/or illustrated in the drawings. The invention is capable of other embodiments or of being practiced or carried out in various ways.

### EXAMPLES

### Materials and Methods

BCP was obtained from Sigma-Aldrich (St. Louis, Mo., USA), catalogue Nr. W225207 (assay not indicated) and further purified using preparative HPLC (HP1090 series; column, PEGASIL ODS (Senshu Sci. i.d. 10×250 mm); solvent, 70% CH3OH; flow rate, 2.0 mL/min; detection, UV 220 nm] to remove other sesquiterpenes. GC-MS analysis showed that the BCP used in the below include:

Batch 1: 95% E-BCP, 3% Z-BCP (a total of 98% BCP), 1% BCP oxide and traces of α-humulene.
In another purification the analysis showed:
Batch 2: Total BCP about 85%, alpha-humulene about 12%, copaene about 1%, eugenol about 0.2%, δ-cadinene about 0.2% and BCP oxide about 0.2% as determined by GC-MS analysis.

AM630 (6-Iodopravadoline, CAS 164178-33-0—a selective CB2 receptor antagonist/inverse agonist) was obtained from Cayman Chemical Company (Ann Arbor, Mich., USA). Phencyclidine (PCP), Cremophor EL and DMSO were obtained from Sigma-Aldrich (St. Louis, Mo., USA).

### Animal Model of Schizophrenia:

The mouse model of schizophrenia was established. Phencyclidine (PCP), an NMDA antagonist which induces schizophrenia and psychotic effects in humans, was administered to murine pups (injection of 5 mg/kg in saline) on about postnatal days 3, 5, 7, 9, 11, 13, and 16. This treatment induces long-lasting schizophrenic-like effects in mice that lasted into adulthood. The therapeutic effects of beta-caryophyllene, a dietary cannabinoid and CB2 receptor selective agonist, or of HU-308, in accordance with the teachings herein were evaluated.

### Example 1

BCP 5 mg/kg Injectable Composition 1:0.6:18.4 Cremophor EL:Ethanol:Saline

BCP (10 mg) was weighed and dissolved in 0.6 ml absolute ethanol (Merck, 100983) under stirring. One ml of Cremophor EL was added while stirring, then 18.4 ml sterile saline was added while continued stirring, to obtain the title composition.

### Example 2

BCP 5 mg/kg in Injectable Composition with DMSO:Cremophor EL:Saline 0.6:1:18.4

BCP (10 mg) was and dissolved in 0.6 ml DMSO under stirring, then Cremophor EL (1 ml) and 18.4 ml sterile saline were added under stirring, to obtain the title composition.

### Example 3

PCP Injectable Composition—5 mg/kg in Saline

PCP powder (10 mg) was weighed in a hood into a sterile Eppendorf and dissolved in 20 ml sterile saline to reach a concentration of 0.5 mg/ml. PCP was injected at 10 μl/g body weight, yielding a dose of 5 mg/kg.

### Example 4

BCP 10 mg/kg in Injectable Oil

Oral BCP composition in commercial canola oil vehicle. BCP (20 mg) was weighed and dissolved in 20 ml canola oil.

### Example 5

### PLGA Based Injectable Depot Particles

Prepare BCP microparticles by solvent extraction/evaporation method by double emulsion. Emulsify a water solution containing 100 mg of beta caryophyllene (BCP) with a solution of 50:50 dichloromethane/ethanol containing 450 mg PLGA. Pour slowly this w/o emulsion into an aqueous solution (200 ml) containing 2% PVA and emulsify using a mechanical stirrer (300 rpm) at 25° C. Evaporate the organic solvent under stirring (100 rpm) for 2 h. Collect the formed microparticles by centrifugation and wash with distilled water to remove excessive emulsifier. Freeze-dry the final suspension to obtain a fine powder.

### Example 6

### Polycaprolactone (PCL) Based Injectable Depot Particles

Prepare PCL based BCP microparticles by solvent extraction/evaporation method by double emulsion. Emulsify a water solution containing 100 mg of beta caryophyllene (BCP) with a solution of 70:30 dichloromethane/acetone containing 800 mg polycaprolactone. Pour slowly the obtained w/o emulsion into an aqueous solution (200 ml) containing 2% PVA, 1% Tween 80 and emulsify using mechanical stirrer (500 rpm) at 25° C. Evaporate the organic solvent under stirring (300 rpm) for 4 h. Collect the formed microparticles by centrifugation and wash with distilled water to remove excessive emulsifiers. Freeze dry the final suspension to obtain a fine powder.

### Example 7

### PLGA Based Implant-Rods

Prepare PLGA based rod-shaped biodegradable implants, 20 mm in length and 2 mm in diameter, by solvent extraction/evaporation method. Pour slowly a solution of 50:50 dichloromethane/ethanol containing 1000 mg PLGA, and 200 mg of beta caryophyllene (BCP) into special rod shaped mold. Evaporate the organic solvent using vacuum oven during 12 h at room temperature.

### Example 8

### Preparation of BCP Tablets

Mix 50 g BCP together with 2 g Vitamin E TPGS, 1 g of mineral oil, 20 ml of ethanol absolute and mix all ingredients until completely dissolved.

Dry the mixture obtained in a fluidized bed.
Add the dry mixture into a second mixture to be prepared from:

### PVP K 30 5 g

### BHT and BHA 0.05+0.05 g

### Starch 1500 3 g

Lactose for direct compression—140 g
Mannitol for direct compression—200 g
Sieve the obtained mixture with 1.4 mm sieve and mix 10 minutes in a planetary mixer.
Add 1 g magnesium stearate to the mixture and mix for 2 minutes.
Transfer the resulting powder into a type B rotary tableting machine with flat round shape 3 mm mini tablets punches and compress into 800 mg tablets containing 100 mg THC/tablet.

### Example 9

### Preparation of Enteric Coated BCP Tablets

Place tablets containing 100 mg BCP per tab in a perforated coating pan and coat with Eudragit L 30D coating layer, 6% w/w. The enteric coated tablets comply with the USP enteric coated solid dosage forms characteristics.

### Example 10

### Preparation of Colonic Delivery Tablets of BCP

Place tablets containing 100 mg BCP per tab in a perforated coating pan and coat with Eudragit S 100 coating layer, 8% w/w. The coated tablets release their content at a pH above 7 which complies with the pH conditions in the terminal ileum and lower parts of the GI tract, the colon.

### Example 11

### Controlled Release BCP Tablets

Mix together 10 gr BCP together with: 60 gr HPMC K4M, 20 gr HPMC E50, 15 gr Klucel
HF. Granulate with 30 gr ethanol absolute.
Dry in fluid bed, 60 c, 45 min.
Screen through 0.85 mm
Mix with 1 gr Syloid 244, 0.5 gr Mg stearate.
Compress into about 1 gr tablets containing 100 mg BCP.

### Example 12

### Controlled Release Enteric Coated BCP Tablets

Place controlled release tablets containing 100 mg BCP per tab in a perforated coating pan and coat with Eudragit L 30D coating layer, 6% w/w. The enteric coated tablets comply with the USP enteric coated solid dosage forms characteristics.

### Example 13

### Extended Release Colonic Delivery BCP Tablets

Place the extended release tablets containing 100 mg BCP per tab in a perforated coating pan and coat with Eudragit S 100 coating layer, 8% w/w. The coated tablets release their content at a pH above 7 which complies with the pH conditions in the terminal ileum and lower parts of the GI tract, the colon.

### Example 14

I. Treatment of Mice with BCP at Postnatal Days 3-16

BCP (final dose 10 mg/kg in 1:0.6:18.4 Cremophor EL:ethanol:saline) was administered by injection 1 hour after PCP. Results were obtained from two different litters, each of which was divided into 3 groups:
Group 1: vehicle (n=6 and 4 pups, respectively);
Group 2: PCP (n=6 and 5 pups, respectively);
Group 3: PCP+BCP (n=5 and 5 pups, respectively).

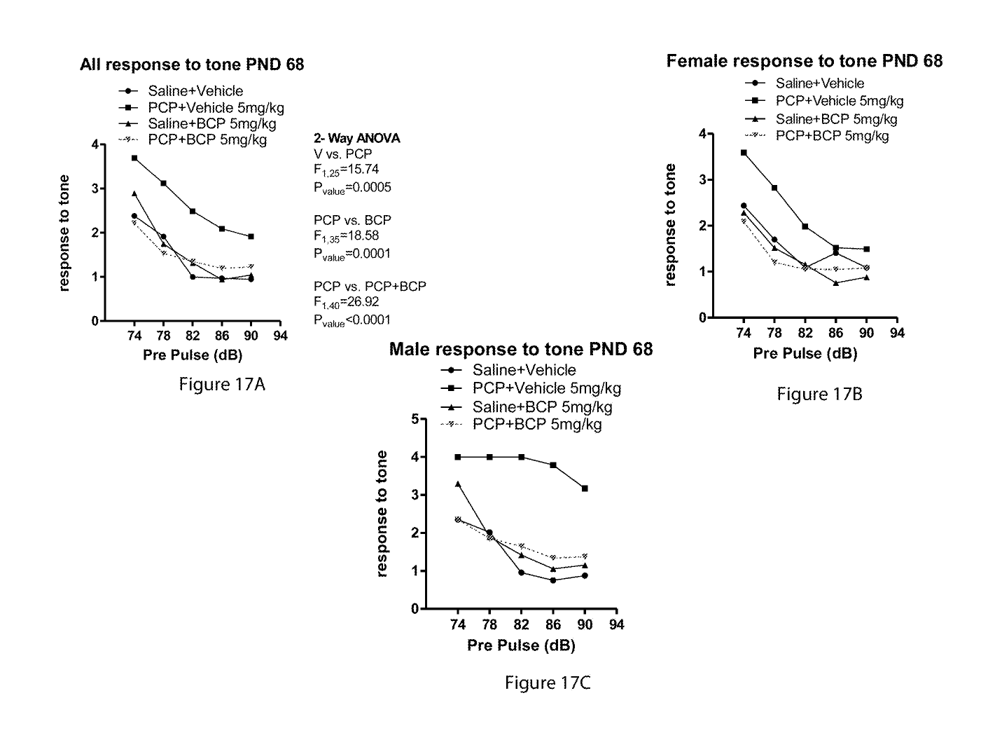

In FIG. 17 BCP (final dose 10 mg/kg in 1:0.6:18.4 Cremophor EL:DMSO:saline) was administered by injection 1 hour after PCP. Results were obtained from one litter which was divided into the three groups.

### Assessment of Positive/Negative Schizophrenic-Like Behavior:

### Open-Field Test (Crossing and Rearing)

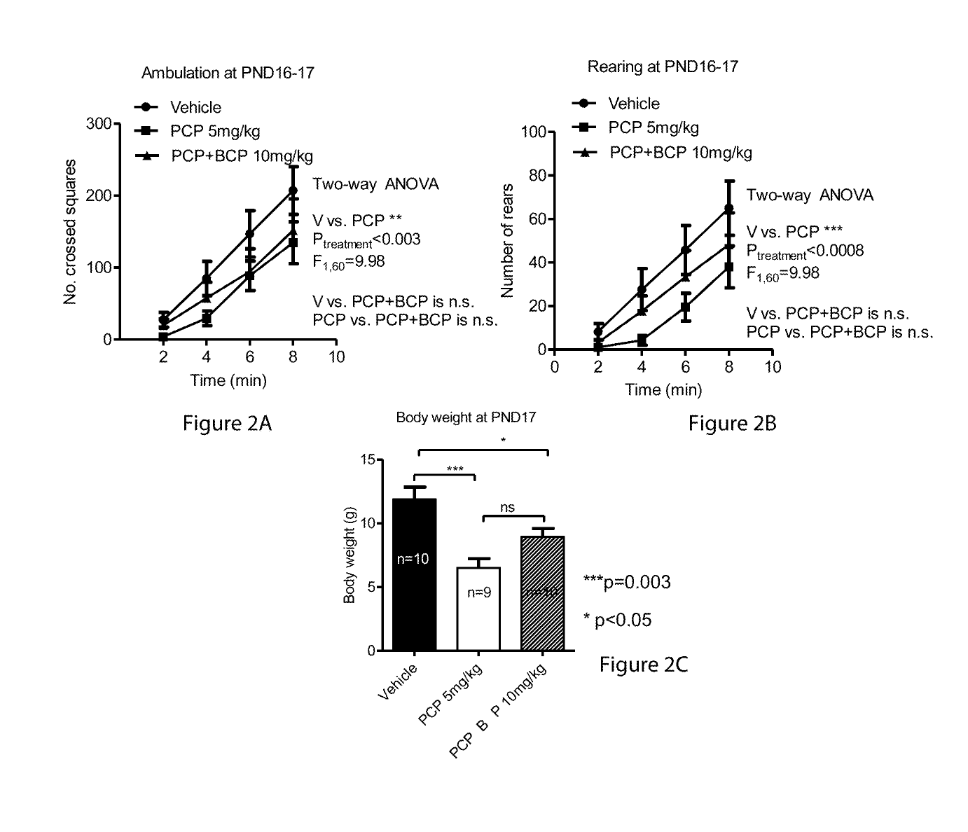

Mice were assessed for hyperactivity behavior on postnatal day 16 (FIG. 2). Mice were placed in the center of a transparent glass cube cage 30×40 X31 cm divided into squares of 7.5×7.5 cm. The number of squares and rearing activity were counted for 8 min.

Positive Symptoms. Prepulse Inhibition (PPI) of the Startle Reflex

In this experimental model, a weak stimulus (74-90 dB tone) inhibited the subsequent response to a strong stimulus (120 dB tone). Reduced prepulse inhibition of the startle reflex (PPI) was taken as an index of the positive symptoms of schizophrenia (Josselyn and Vaccarino, 1998).

PPI was assessed similarly to the method described by Varty et al. In the employed model, mice were placed in a startle chamber and allowed to acclimate for 5 min. A loudspeaker produced a 65 dB background white noise or the various acoustic pre-pulse stimuli (dB): 74, 78, 82, 86, and 90 (20 ms). A 120 dB (40 ms) stimulus was given first to induce a response to startle. The response of the mouse was transduced and stored by a computer. Each test session lasted for 11 min and consisted of 5 presentations of each of the trial types presented in random order and separated by 15 second intervals. The amount of prepulse inhibition was calculated as % PPI=[1−(startle response for prepulse+pulse)/(startle response for pulse alone)]×100.

### Negative Symptoms

Anxiety as measured by the paucity of time spent on the two, anxiety-provoking, open arms (as opposed to the two enclosed arms) of an “Elevated Plus Maze” was used as a parameter of negative symptoms of schizophrenia (Josselyn and Vaccarino, 1998). The plus maze was elevated 50 cm above the table top. Behavior of each mouse was recorded for 5 min by a video camera and scored using the “EthoVision” software (Noldus Information Technology, Wageningen, The Netherlands), measuring the number of entries as well as the amount of time spent in each arm, open or closed. Increased time spent in the closed arms indicated increased anxiety. Increased time spent in the opened arms indicated anxiolytic behavior (reduced stress).

### Cognitive Symptoms

In the PPI test, the mouse is expected to remember the startle noise in order to inhibit its response when it is exhibited with a pre-pulse noise. The % PPI indicates of level of disruption of the sensorimotor gating system in schizophrenia but also serves as an indicator of memory function. The % PPI after administration of PCP and treatment of BCP in the PPI test also serve as an index of memory function, further supporting the function of the sensorimotor system.

### Results

### Body Weight

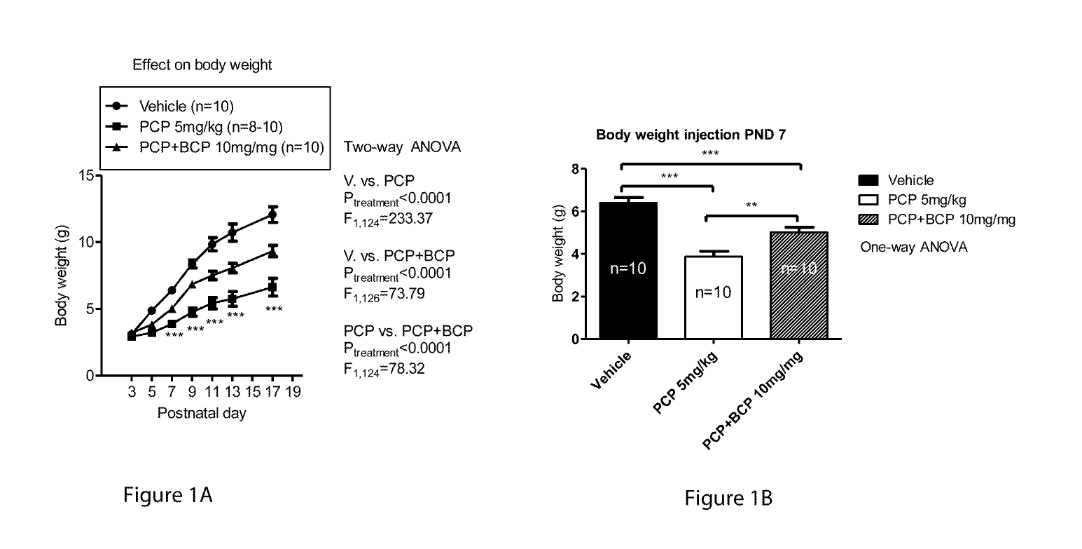

Body weight was recorded at each day of injection (FIG. 1A). PCP was shown to significantly reduce the body weight. The results show that BCP significantly reversed the inhibitory effect of PCP on body weight (p<0.0001 for PCP+BCP vs. PCP).

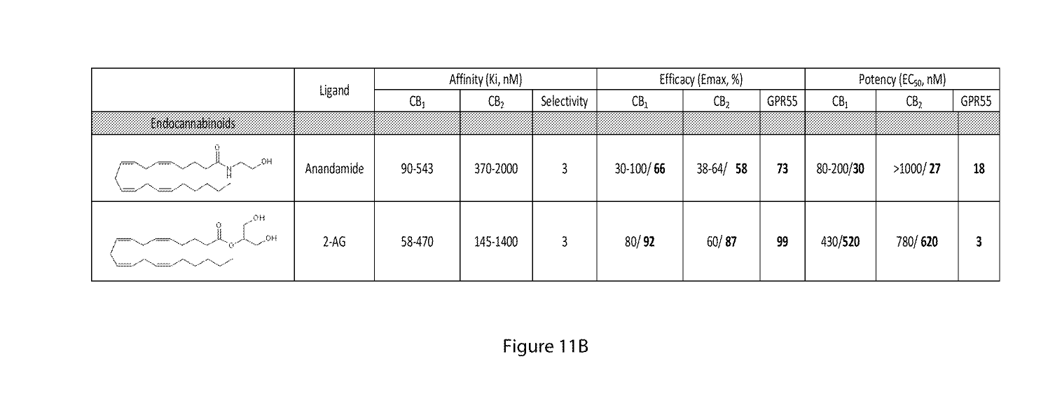

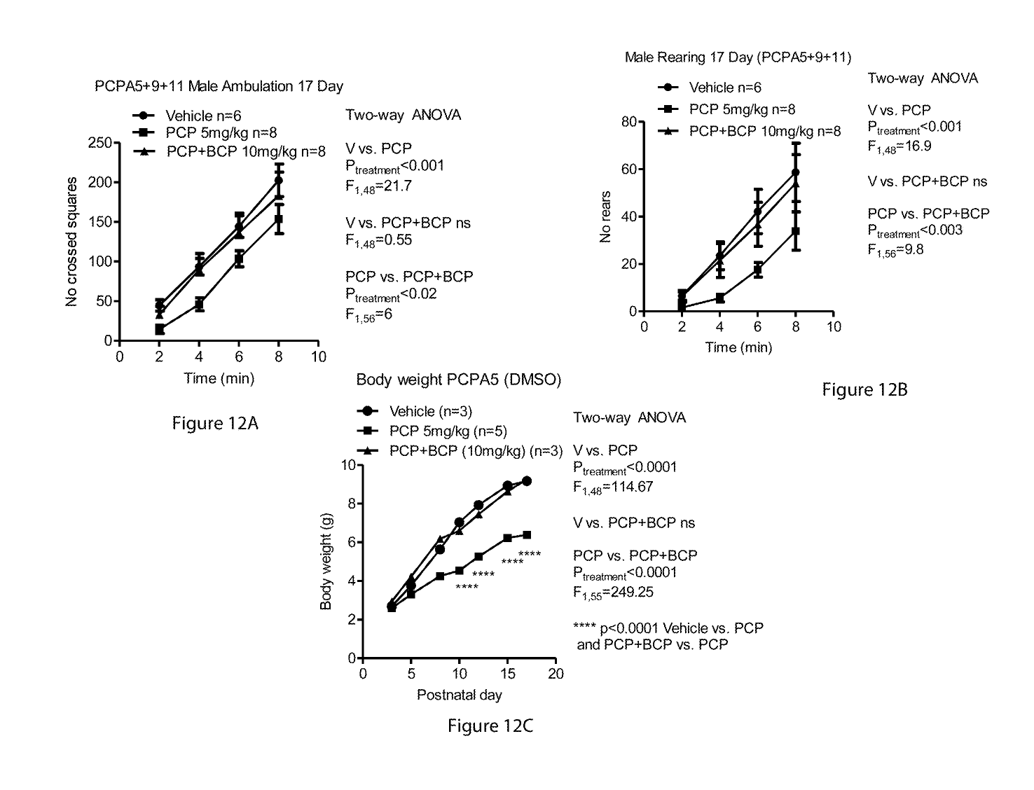

It was shown that at PND 7, PCP induced a significant reduction in body weight. Surprisingly, BCP reversed the reduction in body weight during the first week of life (FIG. 1B) but, under these conditions (injections in ethanol-based vehicle as described above) did not restore the weight completely. However, when vehicle mixture was changed to DMSO-based, BCP completely reversed the effect of PCP on body weight (FIG. 12C).

At postnatal day (PND) 17, the difference between vehicle- and PCP-treated groups in body weight was still significant (p<0.0001) (FIG. 2C). There was no significant difference between groups treated with PCP with or without BCP.

### Rearing and Exploration

At PND 17, at the end of treatment with BCP, locomotor activity, hyperactivity, and exploratory behaviors were tested with the open-field test (FIGS. 2A, 2B). PCP significantly inhibited both ambulation and rearing behaviors. Treatment with BCP reversed the effects of PCP on rearing and exploration. In addition, it is seen that the effect of BCP is not dependent on ethanol as its solvent. It is seen that a DMSO-based carrier completely reversed the effects of PCP on ambulation (FIG. 12A) and rearing behavior (FIG. 12B).

### Results According to Sex

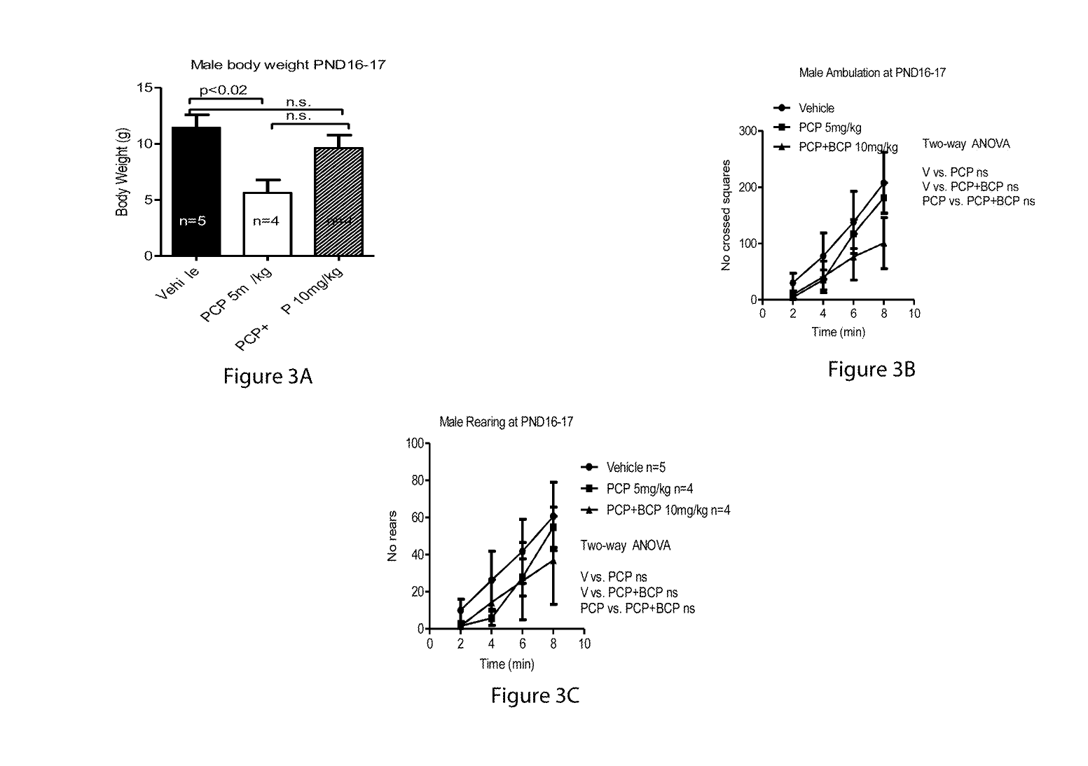

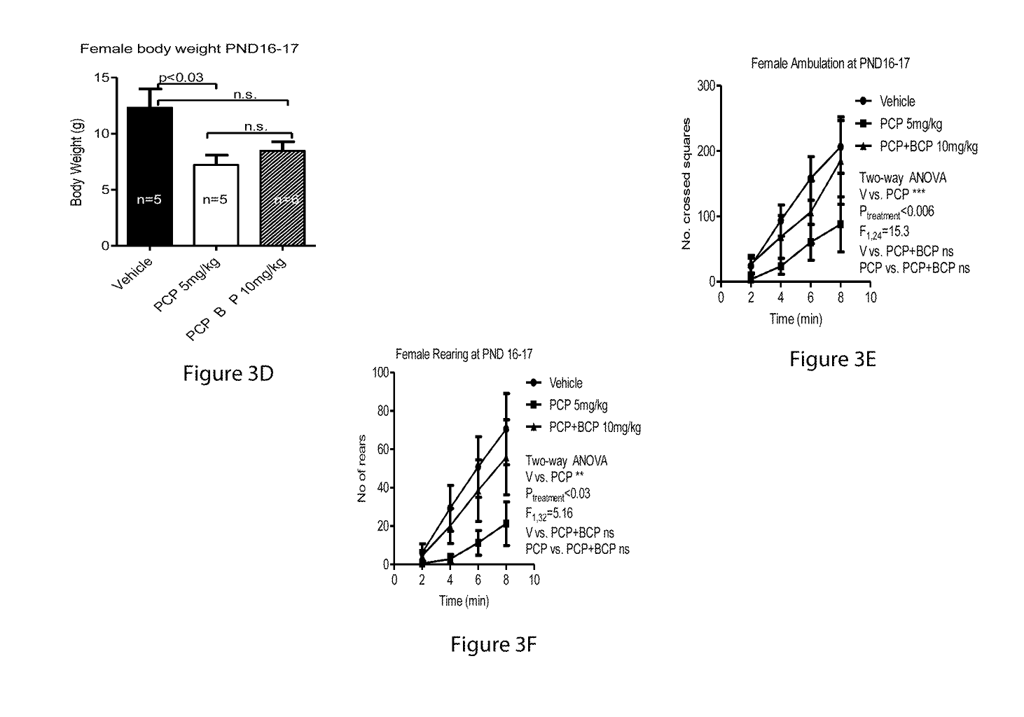

Changes in body weight and results from the open-field test at PND 17 were separated according to the sex of mice (FIGS. 3A-F).

Females appeared to be more sensitive to the effect of PCP (ambulation p<0.006; p<0.03 rearing). Treatment with BCP reversed a proportion of the effects of PCP on ambulation and rearing behavior of female mice (FIGS. 3E, 3F, respectively) but not the effect of PCP on body weight (FIG. 3D). Locomotor activity of male mice appeared to be less sensitive to the treatment of PCP or PCP+BCP (FIGS. 3A-3C). However, the effect of BCP on PCP-induced reduction of body weight in male mice appeared to be more prominent (FIG. 3A).

These results suggest that in some embodiments the administration of BCP for the treatment of schizophrenia has a different effect on male and female mice, being more effective in the treatment of female mice.

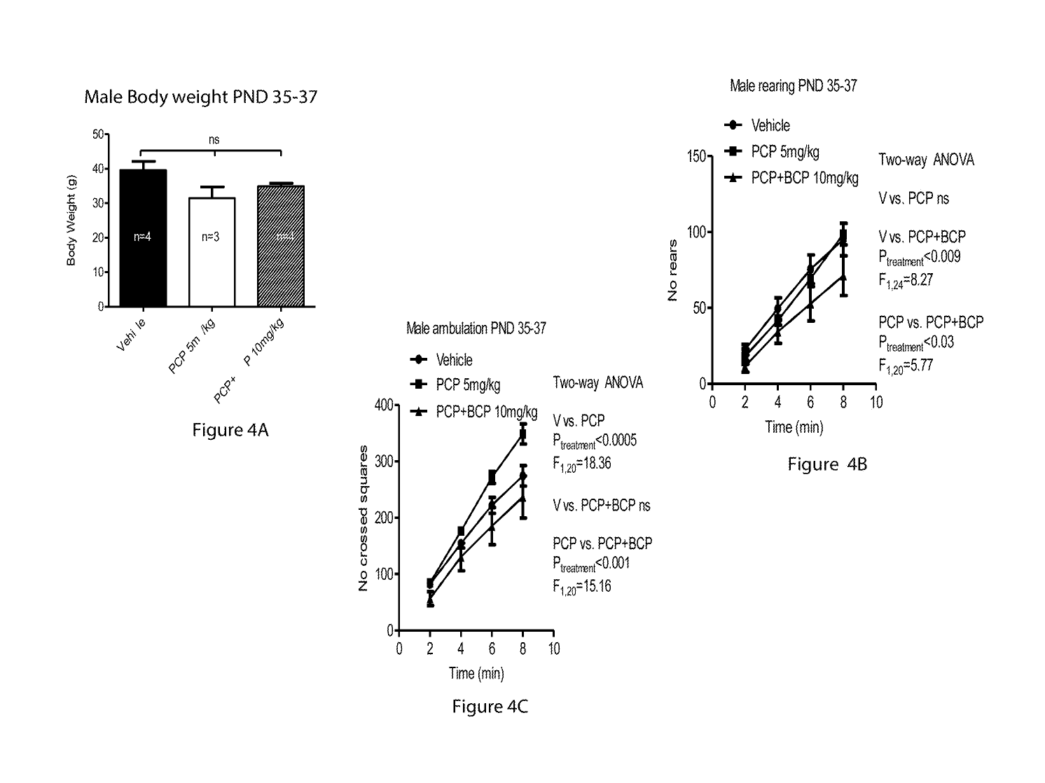

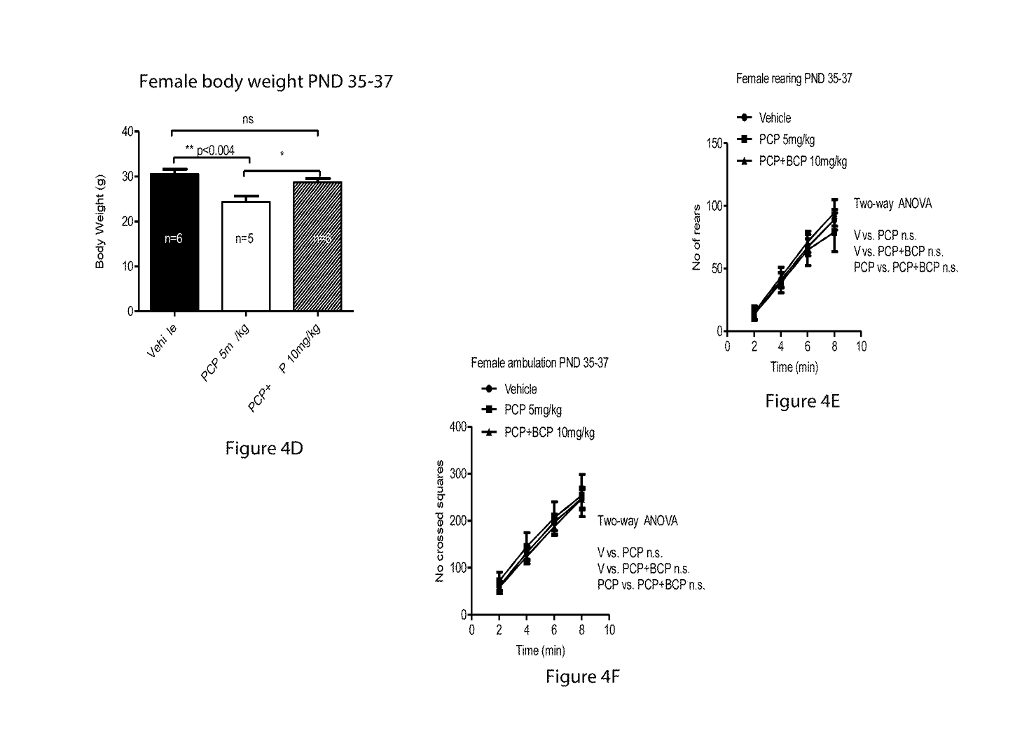

Mice were re-evaluated in the open-field test at PND 35-37. Results of body weight, rearing and ambulation were separated according to the sex of mice (FIGS. 4A-F). Treatment with BCP significantly reversed the effect of PCP on female body weight (FIG. 4D). At this age there was no significant difference in male body weight between the groups (FIG. 4A).

In males, treatment with BCP significantly reduced rearing behavior as compared to vehicle and PCP-treated groups (FIG. 4B).

In males, treatment with PCP significantly increased the locomotor activity compared with vehicle treatment, while administration of BCP reduced locomotor activity (FIG. 4C).

In females, no differences in rearing and exploration behaviors were seen between the different groups (FIGS. 4E, 4F, respectively).

It was concluded that treatment with BCP significantly reversed the effect of PCP in males.

### Prepulse Inhibition Testing

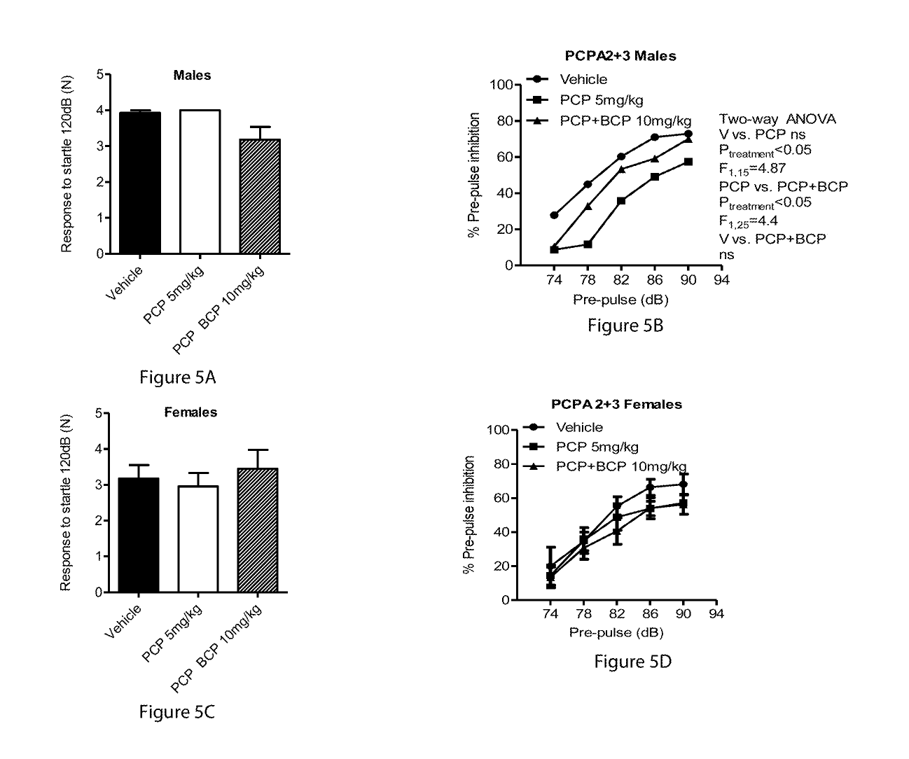

Mice were tested at age 8 weeks in the Pre-Pulse Inhibition test (FIGS. 5A-D).

### Response to Startle

The response to startle stimuli was not significantly different between groups (in males (FIG. 5A) or in females (FIG. 5C). It was concluded that mice in all groups had no problem in hearing and were not apathetic.

### % Inhibition

Males treated with PCP showed a significant reduction in their ability to adjust to sound stimulus as compared to vehicle treated mice. BCP reversed this effect (FIG. 5B).

No significant difference in in the ability to adjust to sound stimulus was seen in females for the PCP-treated group or PCP+BCP treated group (FIG. 5D).

It was concluded that treatment with BCP significantly reversed the effect of PCP on the sensorimotor-gate in males.

### Elevated-Plus Maze Test

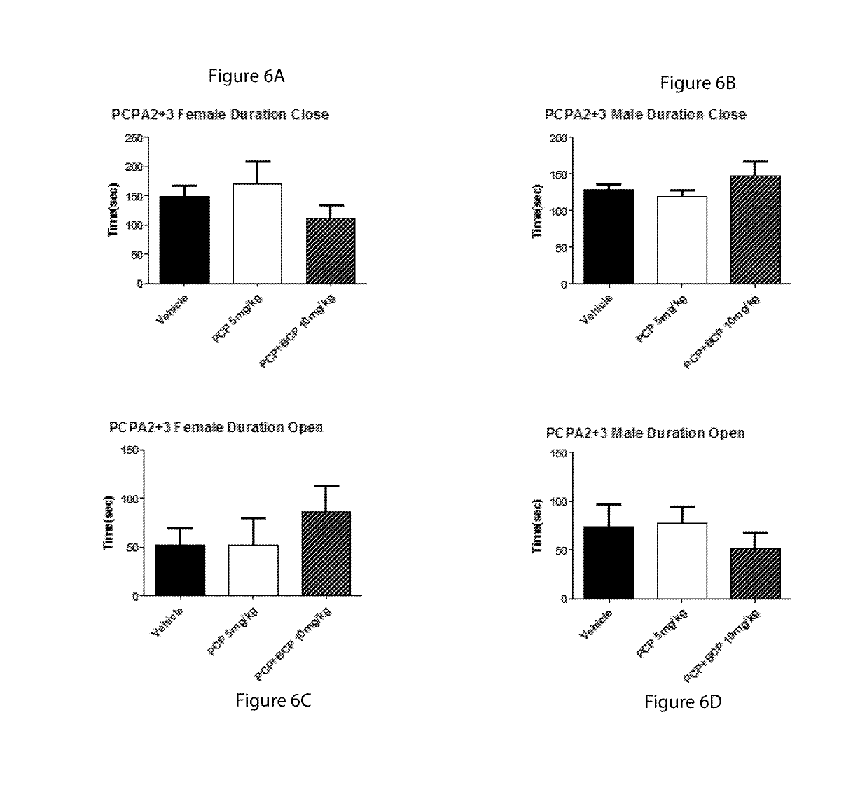

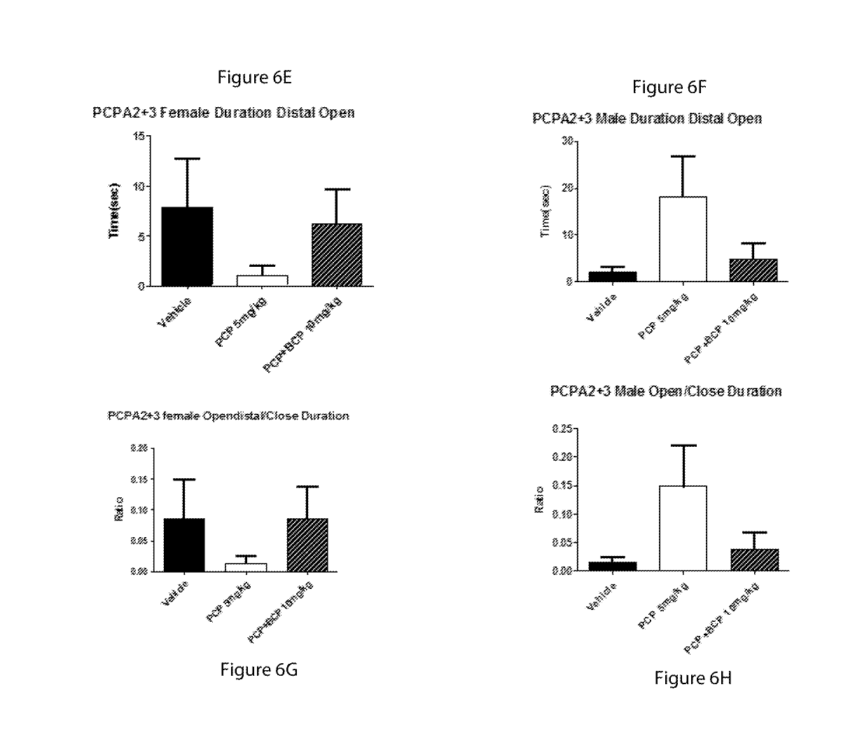

At age 13 weeks old, mice were tested in the Elevated-Plus Maze test which indicates the level of anxiety (FIGS. 6A-H).

Phencyclidine alters the level of anxiety. However, its effect is dependent on the strain of mice, sex and possibly age (Turgeon, 2011; Wily, 1995).

There was no significant change in the time spent in the closed arm or open arm (all length of arm), for either females or males (FIGS. 6A, 6B, 6C, 6D, respectively). PCP reduced the time spent in the distal end of the opened arm (the very far end of the arm from the center) for females, and BCP reversed this effect (FIGS. 6E, 6G). In FIGS. 6F, 6H, PCP increased the time spent in the distal end of the opened arm (the very far end of the arm from the center) for males, and BCP reversed this effect.

It was concluded that PCP induced anxiety in female mice and anxiolytic effect in male mice. BCP reversed both effects.

Expression Level of CB1 and CB2 Receptor in Mice at 9 Days Old (mRNA) or 2 Weeks Old (Protein)

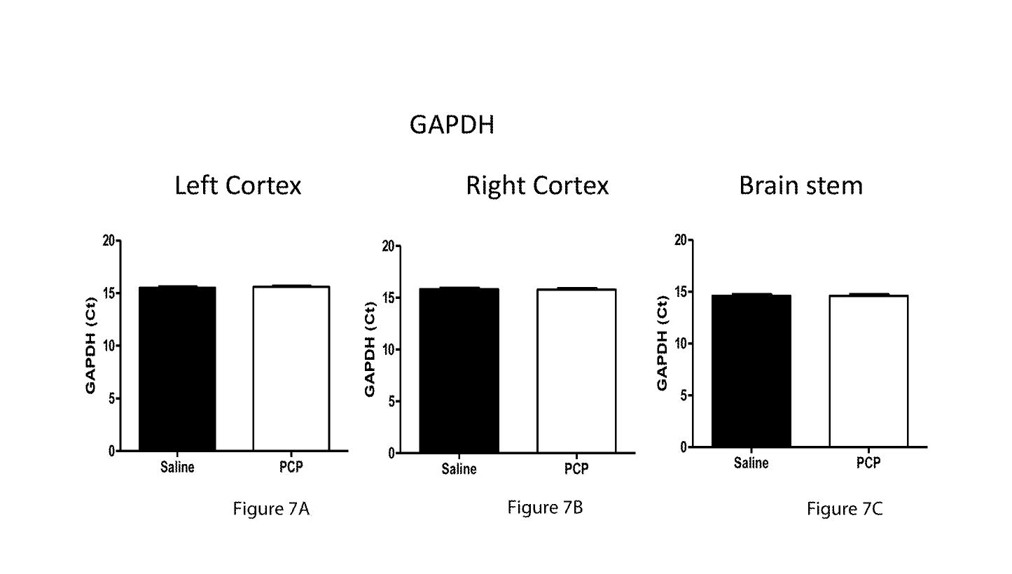

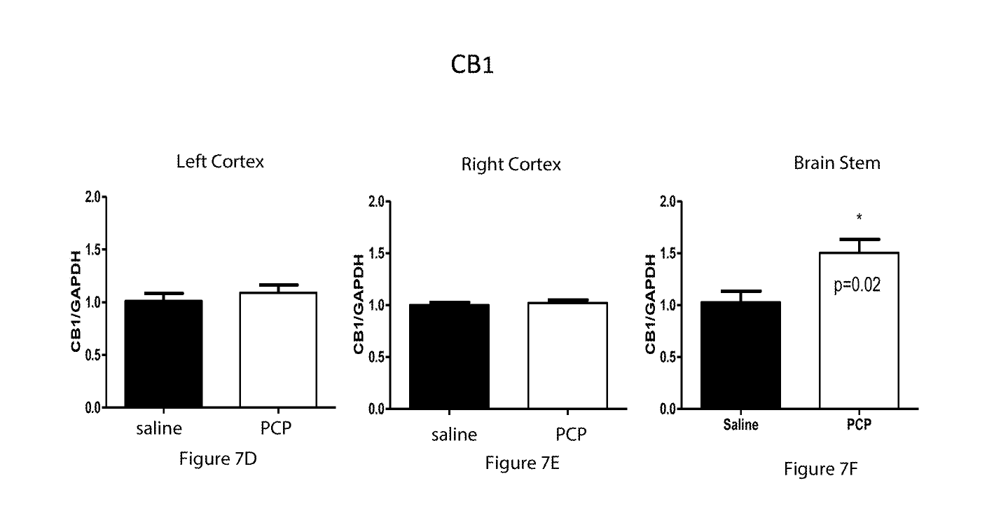

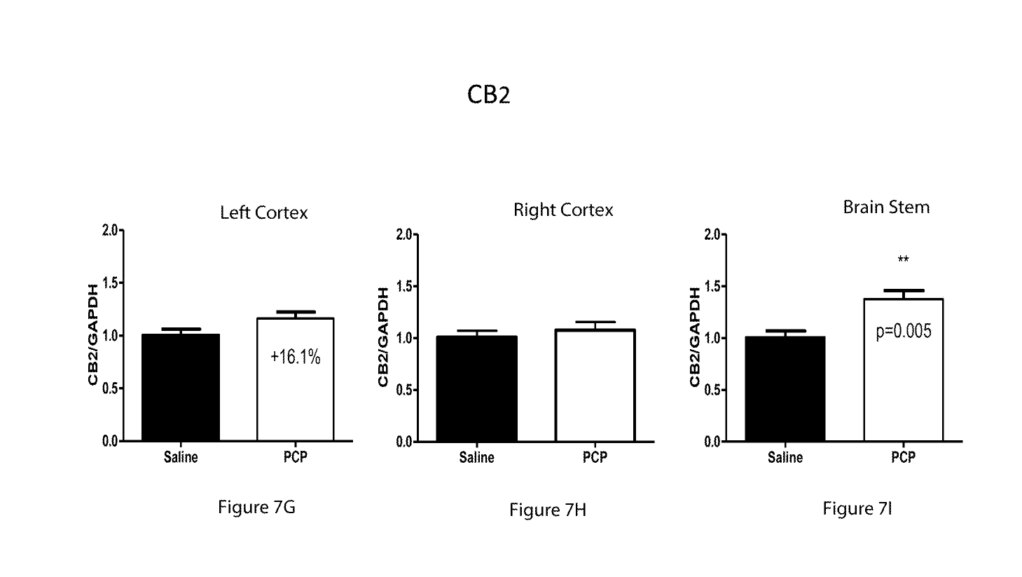

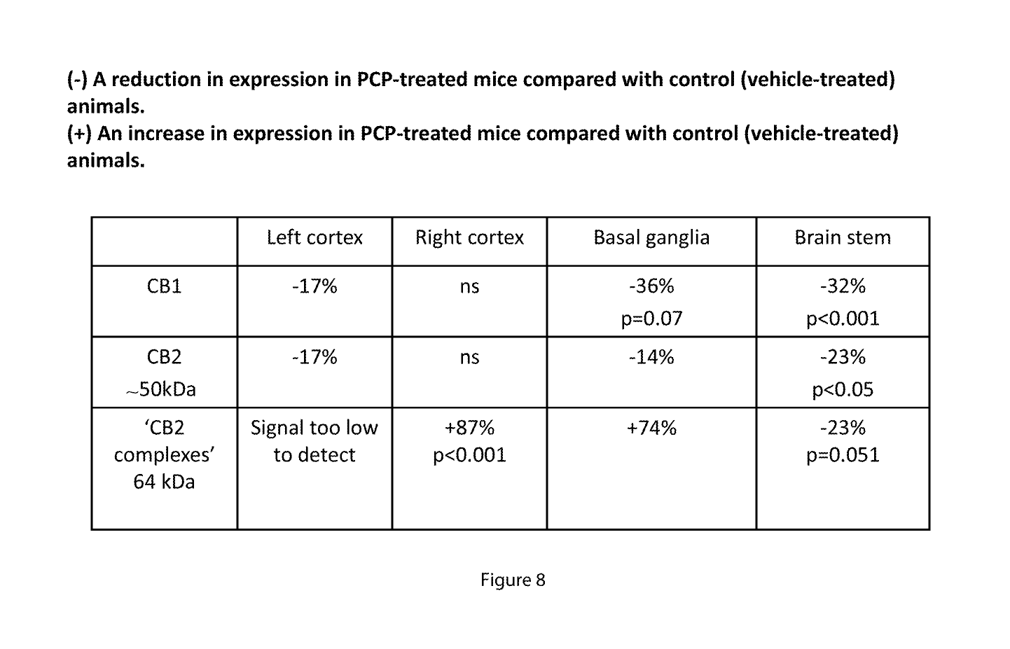

Brain tissue of control mice (saline treated) and mice treated with PCP were analyzed in the left and right cortex and brain stem (FIGS. 7A-I; FIG. 8. Results from the cortex and brain stem were reported by the inventors in 2011 (Anavi-Goffer et al).

No difference in GAPDH was seen in the left cortex, right cortex or brain stem (FIGS. 7A-7C, respectively).

No significant increase in mRNA expression of CB1 receptor was found in the left or right cortex of the PCP-treated mice (FIGS. 7D, 7E, respectively). A significant increase in mRNA expression of CB1 receptor was found in the brain stem of the PCP-treated mice (FIG. 7F).

A significant increase in mRNA expression of CB2 receptor was found in the left cortex of the PCP-treated mice (FIG. 7G) but in the right cortex there was no difference between control and PCP-treated mice (FIG. 7H). A significant increase in mRNA expression of CB2 receptor was found in the brain stem of the PCP-treated mice (FIG. 7I).

It is noteworthy that these results are the opposite of those observed in Western blotting, (FIG. 8) being about 50 kDa (the predicted molecular weight of the CB2 receptor) but in line with these at 64 kDa, suggesting that the CB2 receptor may form a complex with another protein X. This suggests that the change in cannabinoid receptor expression is specific (as no change was found for GAPDH or actin). This also suggests a malfunction in the regulation of cannabinoid receptor synthesis, leading to the accumulation of cannabinoid receptor mRNA in the left cortex and brain stem. On the other hand, synthesis in the right cortex appeared to lead to the formation of CB2 receptor-protein X complexes.

Expression Level of GAD67 in Mice at 9 Days Old (mRNA) or 2 Weeks Old (Protein)

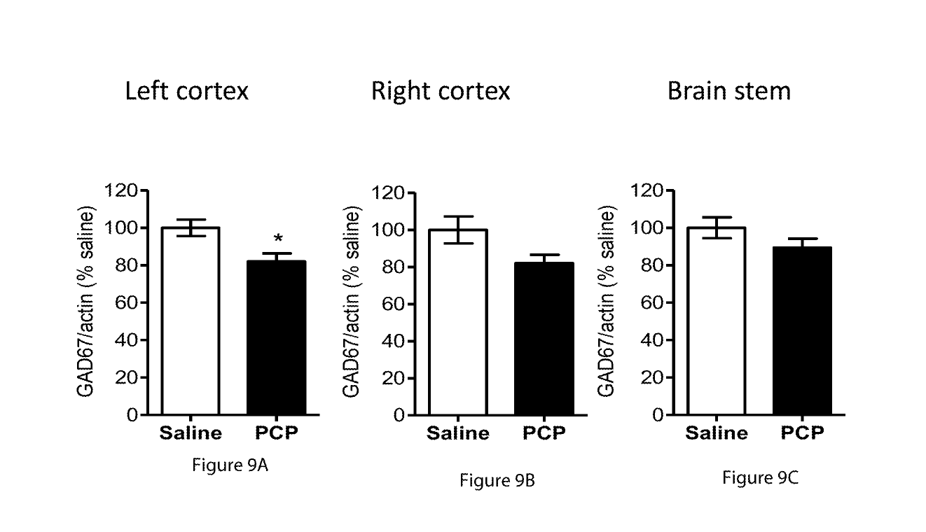

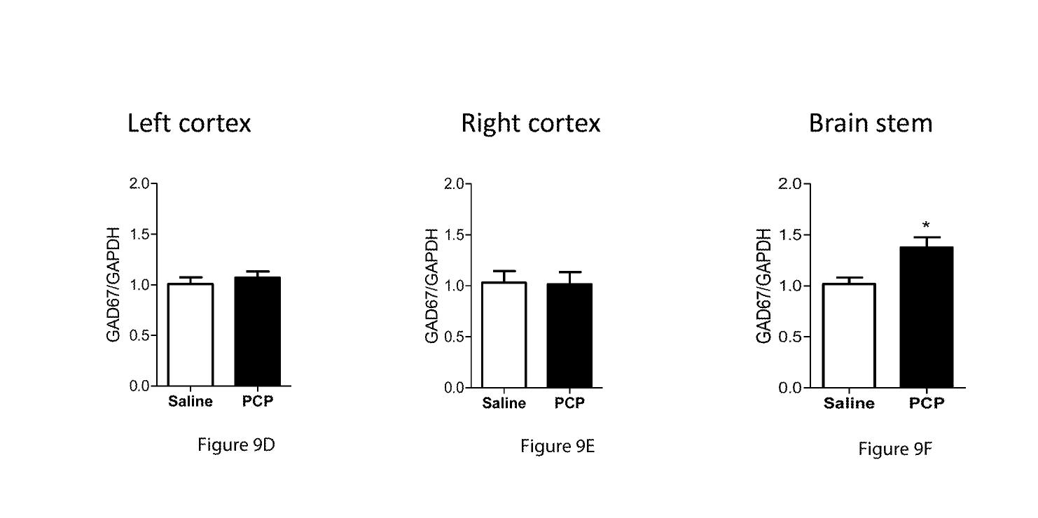

Brain tissue of control mice and mice treated with PCP were analyzed for GAD67, a neurochemical marker for schizophrenia (FIG. 9A-F). In the left cortex, GAD67 protein level was significantly decreased (FIG. 9A) but no change was found at the mRNA level FIG. 9D). In the right cortex, the reduction in protein level of GAD67 did not reach a significant level and no change was seen in the mRNA level (FIG. 9B, FIG. 9E). In the brain stem, GAD67 mRNA level was increased in the PCP-treated group (FIG. 9F), although the results of Western blotting showed a non-significant reduction in GAD67 protein level (FIG. 9C). This suggests that there might be a common mechanism which links the changes in GAD67 to those of CB1 and CB2, and this mechanism may be related to the function of GABAergic neurons.

### Expression of MGL in 2 Week Old Mice

Brain tissues were analyzed for MGL, an enzyme which degrades 2-AG an endocannabinoid (FIGS. 10A-C, FIG. 11B) in control mice and mice treated with PCP. mRNA levels of MGL decreased in the left cortex of the PCP-treated group (FIG. 10A), but not in the right cortex (FIG. 10B). The direction of these results was correlated with the reduction in protein level of MGL as analyzed with Western blotting (FIG. 10C). These results support the requirement for combination of BCP with enzymes inhibitors or enhancers for the treatment of schizophrenia, respectively to expression level.

A scheme of synthesis and degradative enzymes of the endocannabinoid system is shown in FIG. 11A (FIGS. 11A and 11B were published by Anavi-Goffer & Mulder, Chembiochem. 2009 10:1591-8).

### Example 15

II. Postnatal Induction of Schizophrenia (Days 3-15) Followed by Treatment of Adolescent Mice with BCP (Postnatal Days 43-61)

### Methods

PCP 5 mg/kg was administered by injection on PND 3, 5, 7, 10, 12, 13, 15 and 17. Body weight was measured at every injection between PND 3-17. The open field test was conducted on PND 16.

BCP (5 mg/kg in a mixture of DMSO:Cremophor EL:saline 0.6:1:18.4) was injected twice a week (on Sunday and Wednesday) to adolescent mice (PND 43-61) for 3 weeks, a total of 6 injections. After the final BCP injection, mice were tested in the open field test (PND 63), Elevated Plus Maze test (PND 64), PPI test (PND 68) and behavior at the Phenotyper cage (PND 91). Mice were re-tested at adulthood on PND 104 (open field), PND 105 (Phenotyper) PND 106 (PPI).

### Body Weight

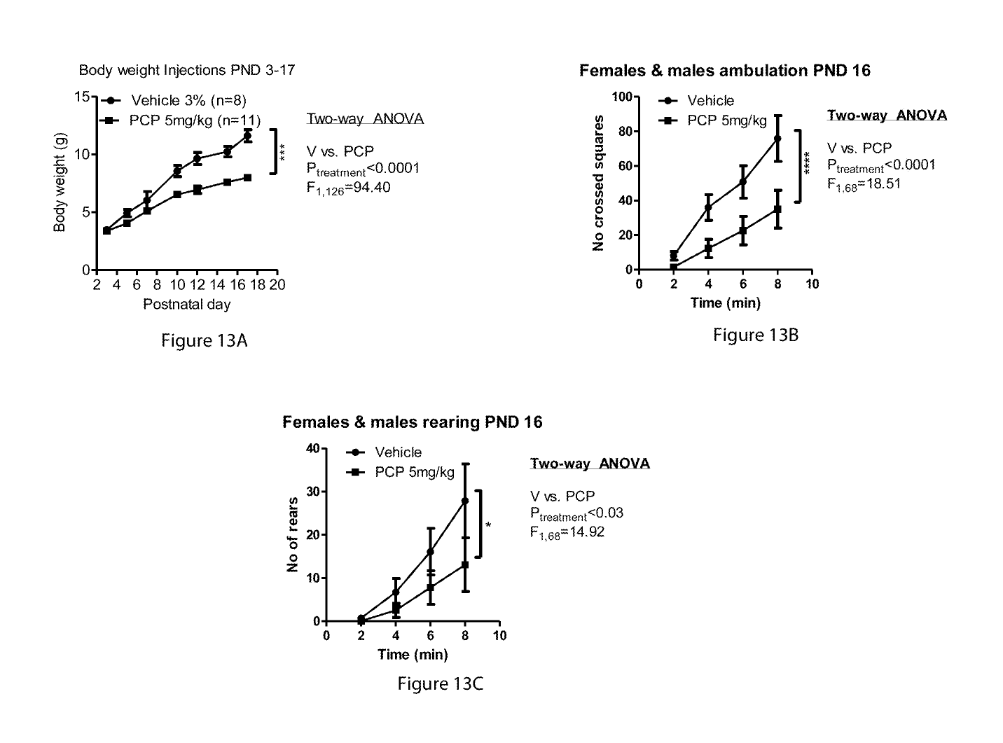

As seen in FIG. 13A, PCP significantly reduced body weight in male and female mice as measured on days 3, 5, 7, 10, 12, 15 and 17.

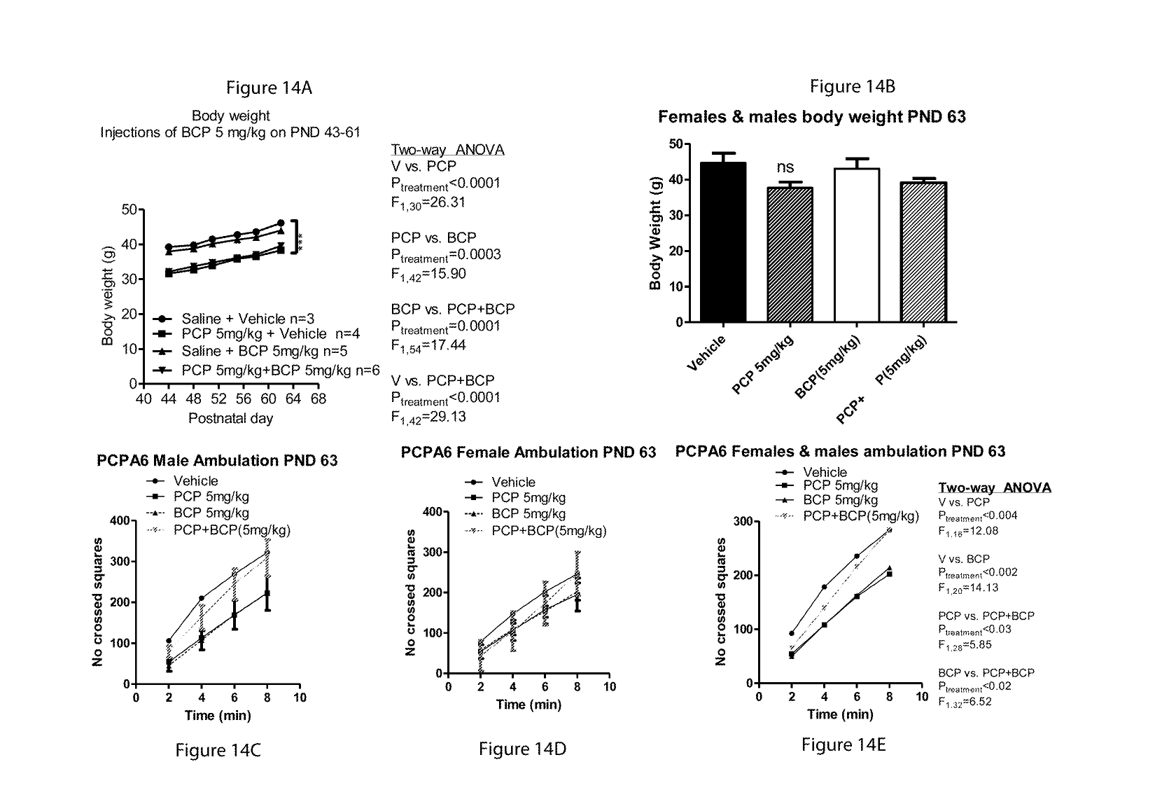

As expected, injections of BCP (5 mg/kg) on PND 43-61 did not affect body weight of adolescent mice (FIG. 14A). At age PND 63 there was no significant difference in the body weight between vehicle-treated mice and PCP-treated mice (FIG. 14B). These results further support the results of Hanus et al, 1999 that HU-308, another CB2 receptor selective agonist, does not modulate body weight of adult mice.

### Ambulation, Rearing

At PND 16, PCP significantly inhibited ambulation, rearing behaviors (FIGS. 13B, 13C).

Surprisingly, BCP treatment during adolescence significantly reversed the effect of postnatal treatment of PCP on ambulation in male and female mice at PND 63 (FIGS. 14C-14E). BCP treatment in adolescence significantly inhibited the exploration of mice compared with saline-treated mice. BCP reversed the effect of PCP on rearing in both females and males (FIGS. 15A-15C). These results are in contrast to Hanus et al., 1999 who showed no effects of HU-308 in the open field assay at a similar age of mice.

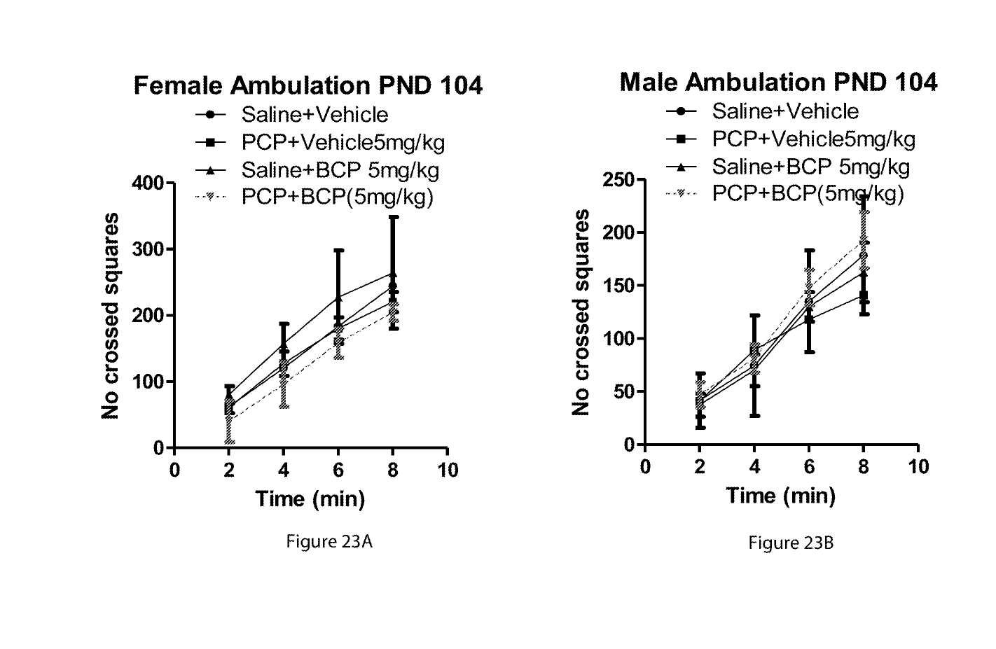

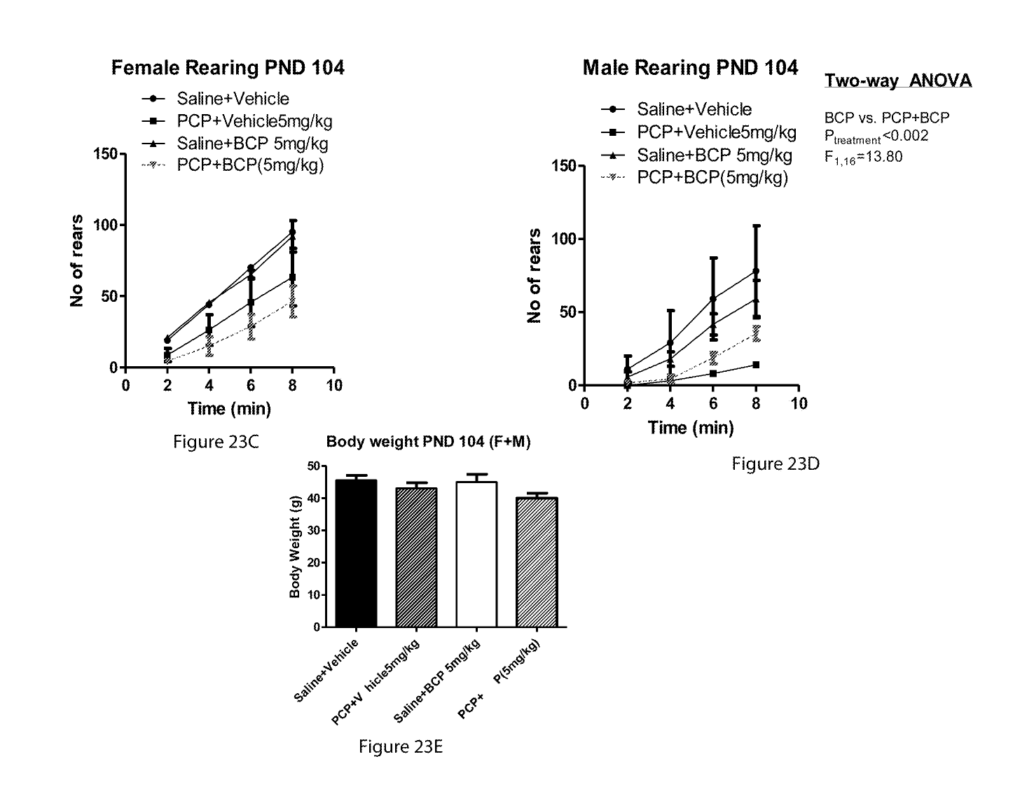

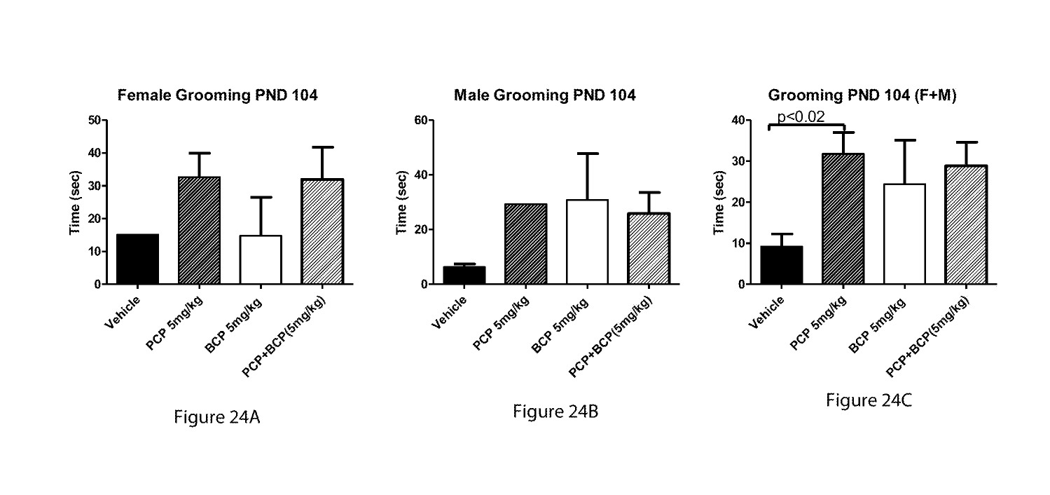

On PND 104 a relapse in exploratory behavior was evident in the schizophrenic female mice that had been treated with BCP (last BCP treatment was on PND 61), as seen in FIGS. 23A, 23C. Rearing behavior of male PCP-treated mice that had been treated with BCP was still significantly higher than that of PCP-treated mice with no BCP treatment (FIGS. 24B, 24C). There was no difference in body weight between groups (FIG. 24E). BCP treatment did not increase body weight in females and males, rather reduced body weight of PCP+BCP treated group (vehicle vs. PCP+BCP, P=0.06).

BCP treatment in adolescence did not reverse the effect of PCP on self-grooming (total, without stimuli) in females or males at PND 104 (FIGS. 24A, 24B, 24C).

### Prepulse Inhibition and Startle Response

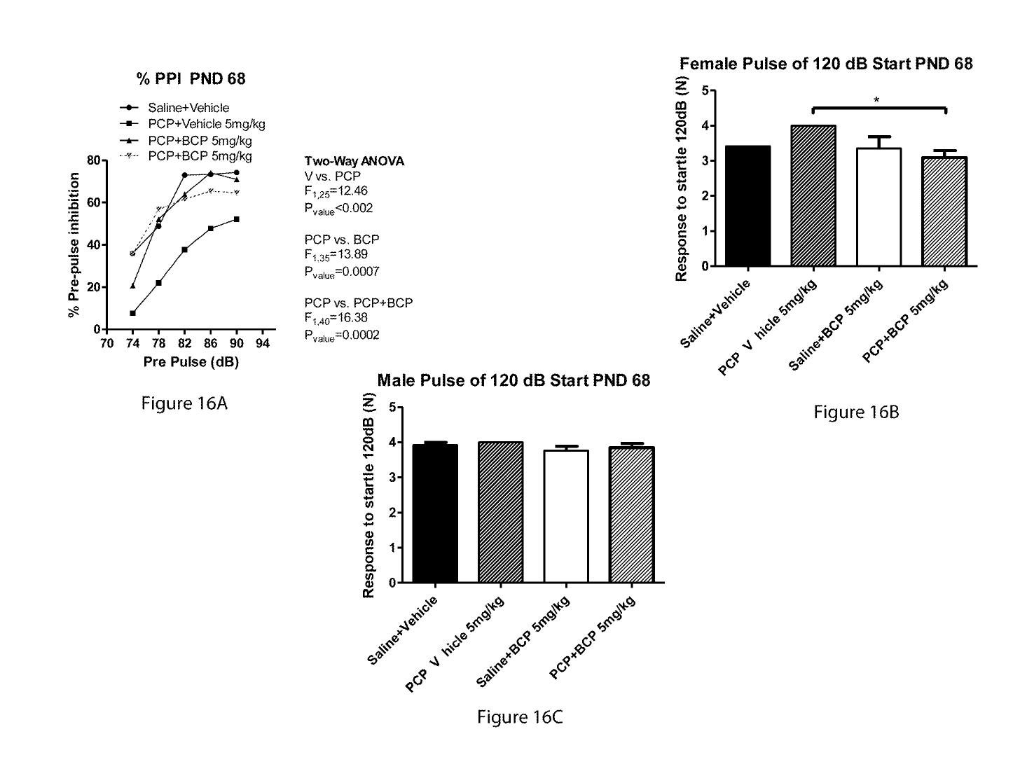

BCP treatment at adolescence significantly reversed the effect of PCP on pre-pulse inhibition (FIG. 16A). BCP treatment at adolescence had no substantial effect on saline-treated mice. BCP treatment at adolescent significantly reversed the effect of PCP on startle response in females (FIG. 16B). In males, there was no difference in the response to the startle stimuli between groups (FIG. 16C).

BCP treatment at adolescence significantly reversed the effect of PCP on response to pre-pulse tones (FIGS. 17A-17C). BCP treatment at adolescence had no effect on saline-treated mice.

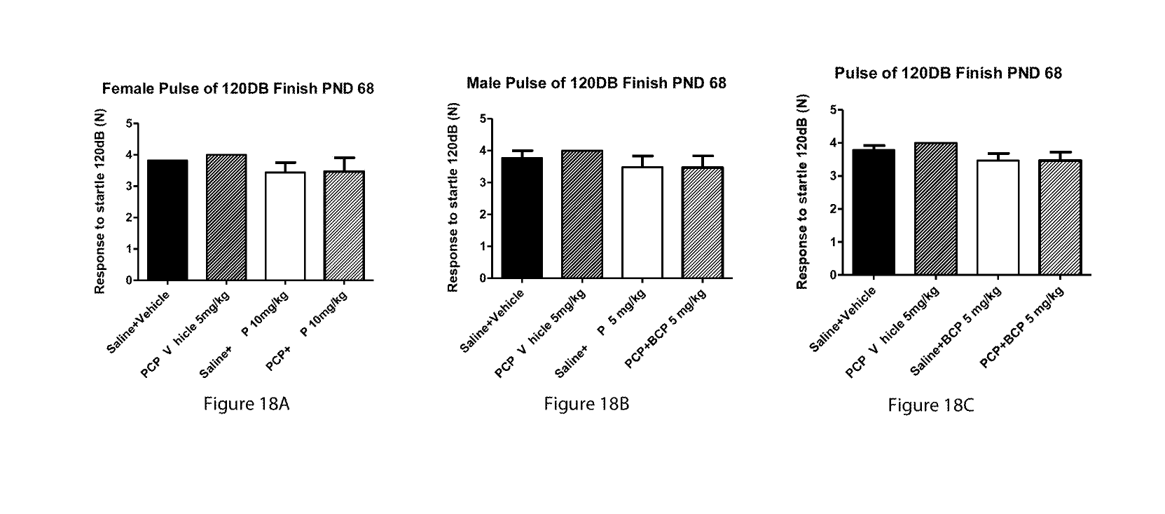

There was no difference between groups in the response to the startle stimuli at the end of the PPI test (FIGS. 18A-18C). BCP treatment at adolescence did not affect the response to startle at the end of the PPI test (FIGS. 18A-18C).

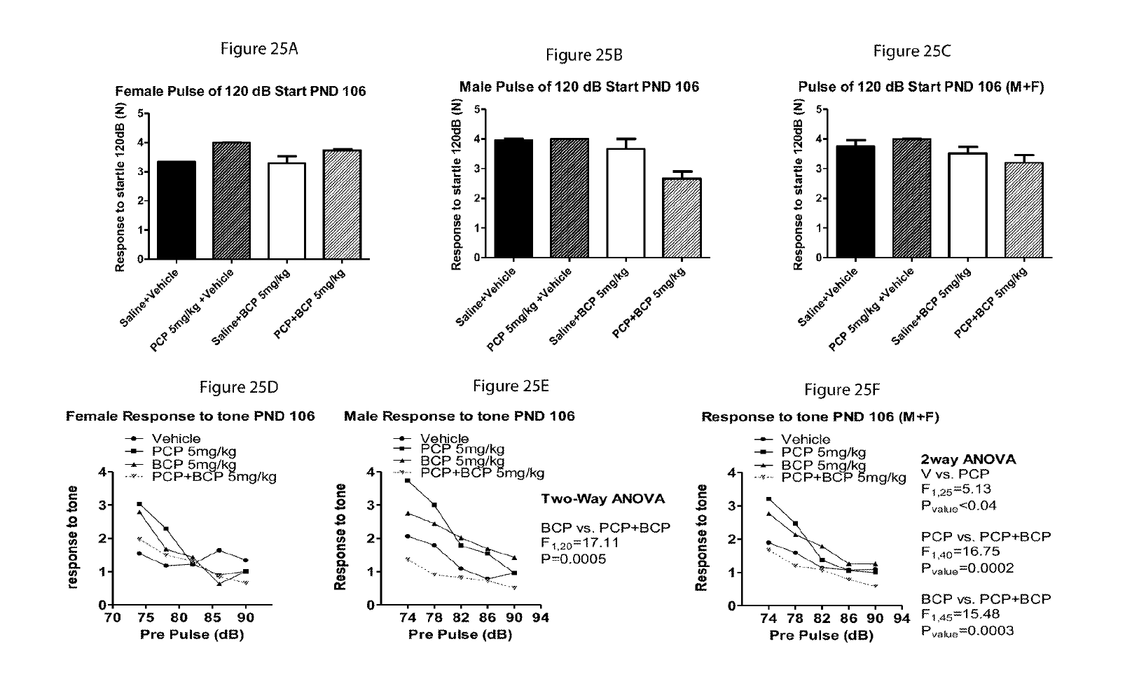

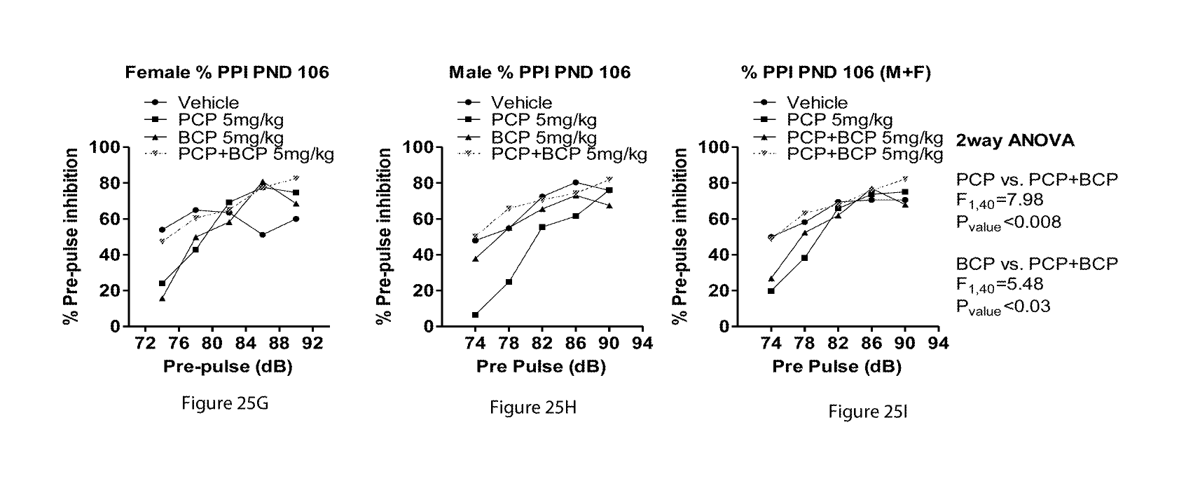

At PND 106, there was no difference in the response to the startle stimuli (120 dB) between groups (FIGS. 25A-25C). BCP reversed the effect of PCP on the response to tones (FIGS. 25D-25F). BCP treatment at adolescence reversed the effect of PCP on the % pre-pulse inhibition (PPI) (FIGS. 25G-25I). As PPI reflex requires functional memory and learning processes, these results support treatment of cognitive deficits associated with mental disorders.

### Plus-Maze Test

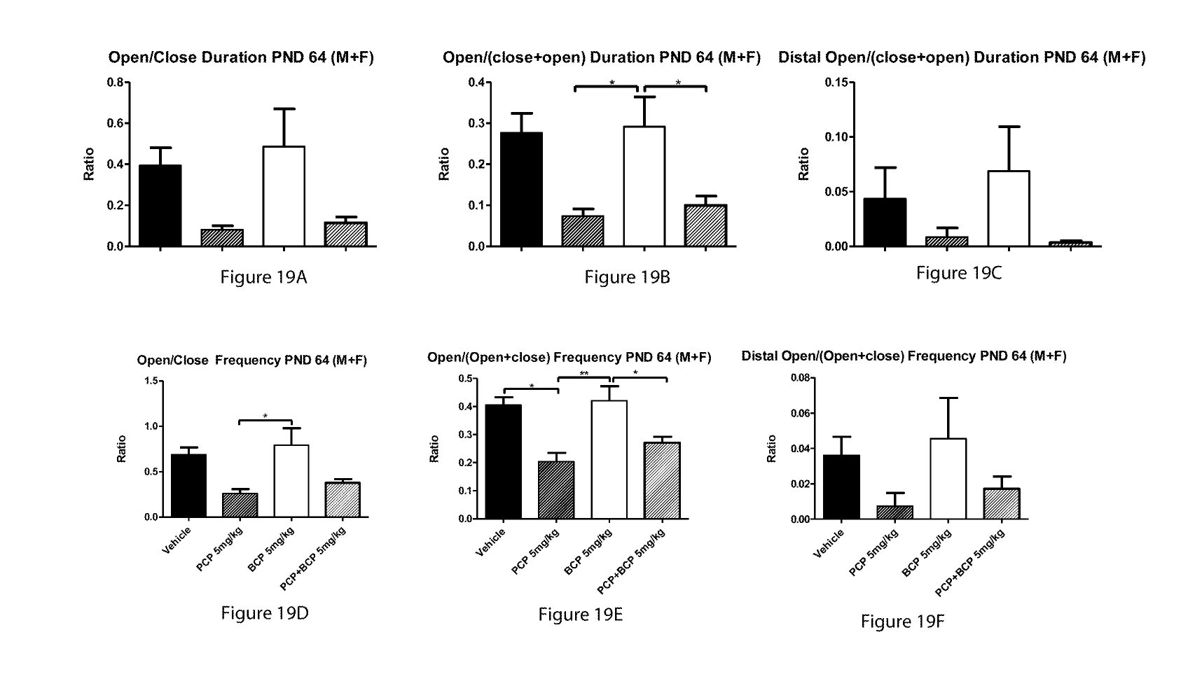

BCP treatment of adolescent mice significantly reversed the effects of PCP on behavior in the close arm of the Elevated Plus Maze at age 64 days (6 weeks). BCP treatment at adolescence significantly reversed the effects of PCP on behavior in the open arm of the Elevated Plus Maze at age 110 days (PND 110). These results further support treatment of anxiolytic and depressive deficits associated with mental disorders. However when traditionally calculated according to open/close or open/(close+open) there is no effect at age 64 (FIGS. 19A-19F).

### PhenoTyper Test

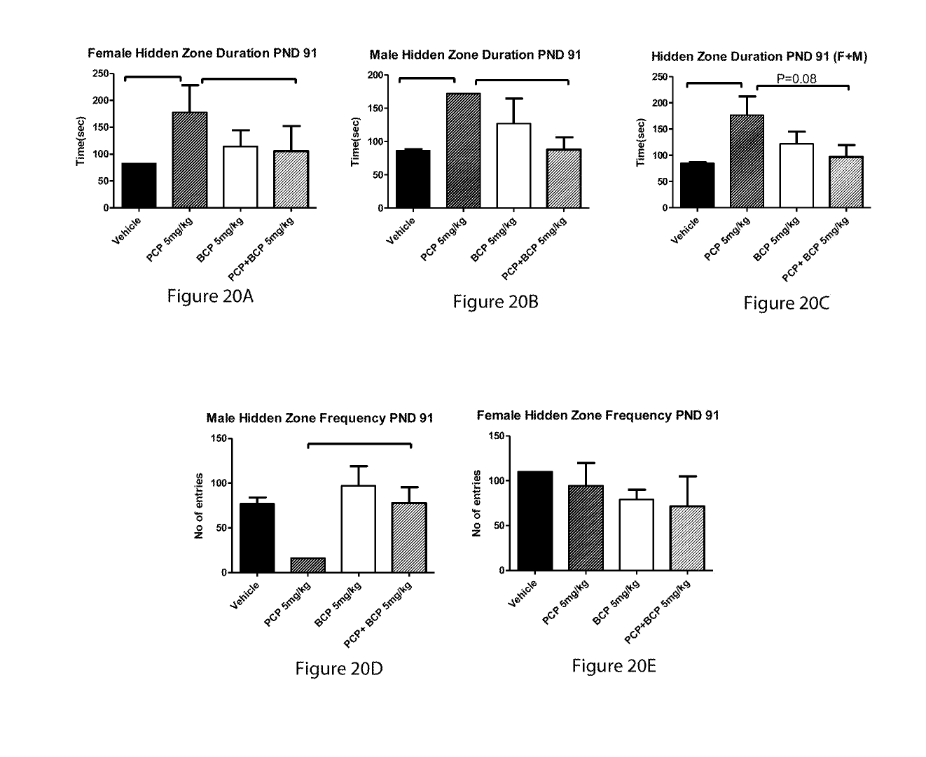

At PND 91, PCP increased the time spend at the Hidden Zone, indicating the PCP-treated mice had higher level of anxiety of mice at the PhenoTyper cage compared with vehicle-treated mice. BCP treatment at adolescence reversed the effects of PCP on the time spend in the Hidden Zone of PhenoTyper cage in females and males, respectively (FIGS. 20A, 20B). FIG. 20C shows combined results, suggesting that BCP reversed the effect of PCP on anxiety level. PCP appeared to reduce the frequency of entries to the Hidden Zone in males (FIG. 20D) but not in females (FIG. 20E). BCP reversed the effect of PCP in males (FIG. 20D).

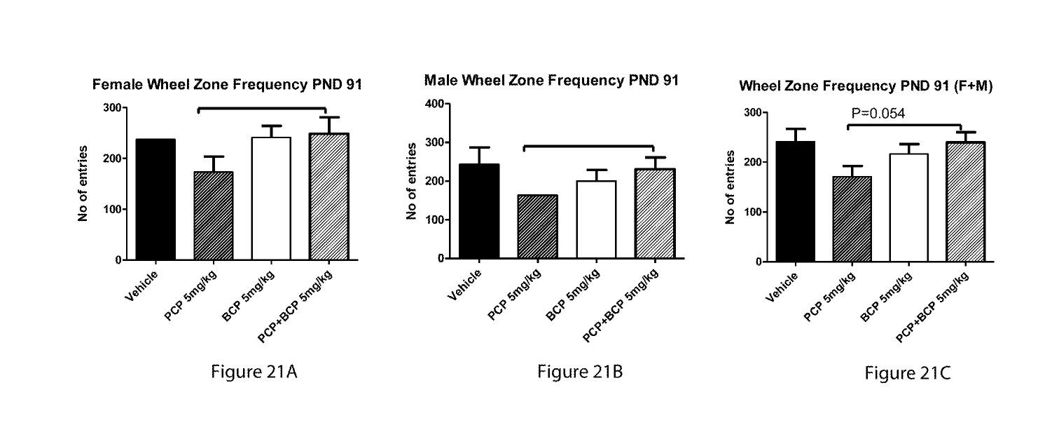

PCP reduced the frequency of entries to the wheel. BCP treatment at adolescence reversed the effects of PCP on the frequency of entries to the wheel in the Phenotyper cage both in females and males (FIGS. 21A, 21B). FIG. 21C, shows combined results of both sex. These results indicate that BCP reversed the action of PCP on loco/motor behavior.

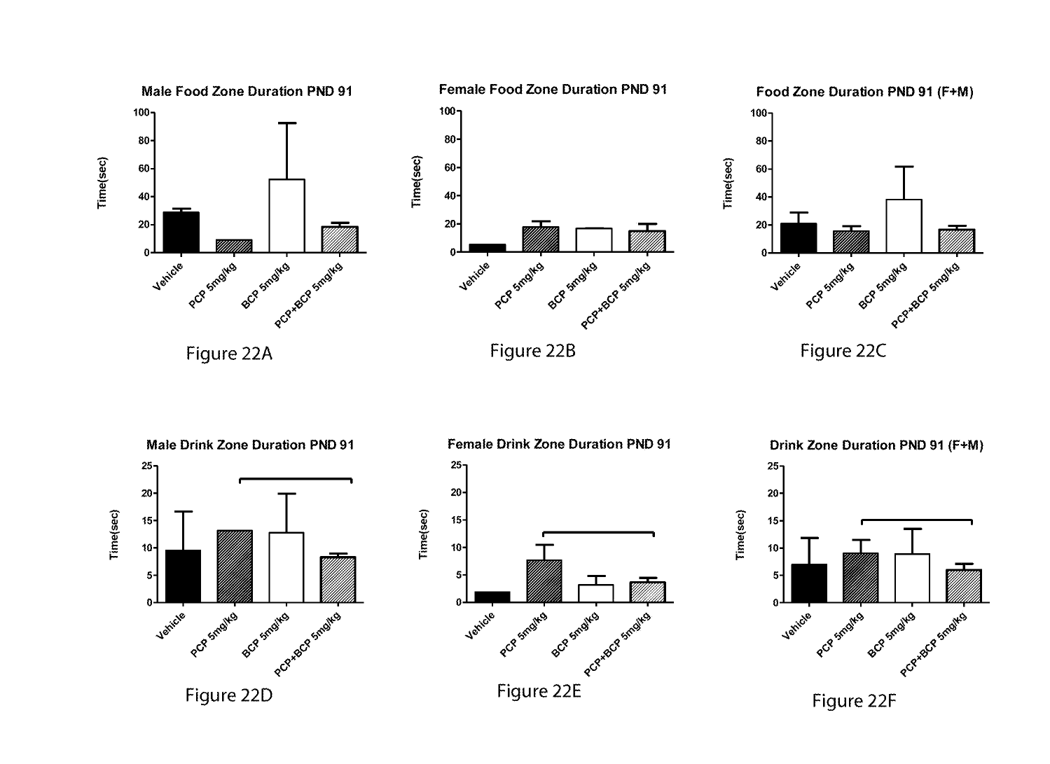

BCP treatment at adolescence did not affect the time spend in the food zone (FIGS. 22A, 22B, 22C).

Compared with PCP-treated group, BCP treatment at adolescence appeared to reduce the time spend at the drinking zone (FIGS. 22D, 22E, 22F).

At PND 105, PCP increased the frequency of entries to the Hidden Zone, indicating an increased level of anxiety.

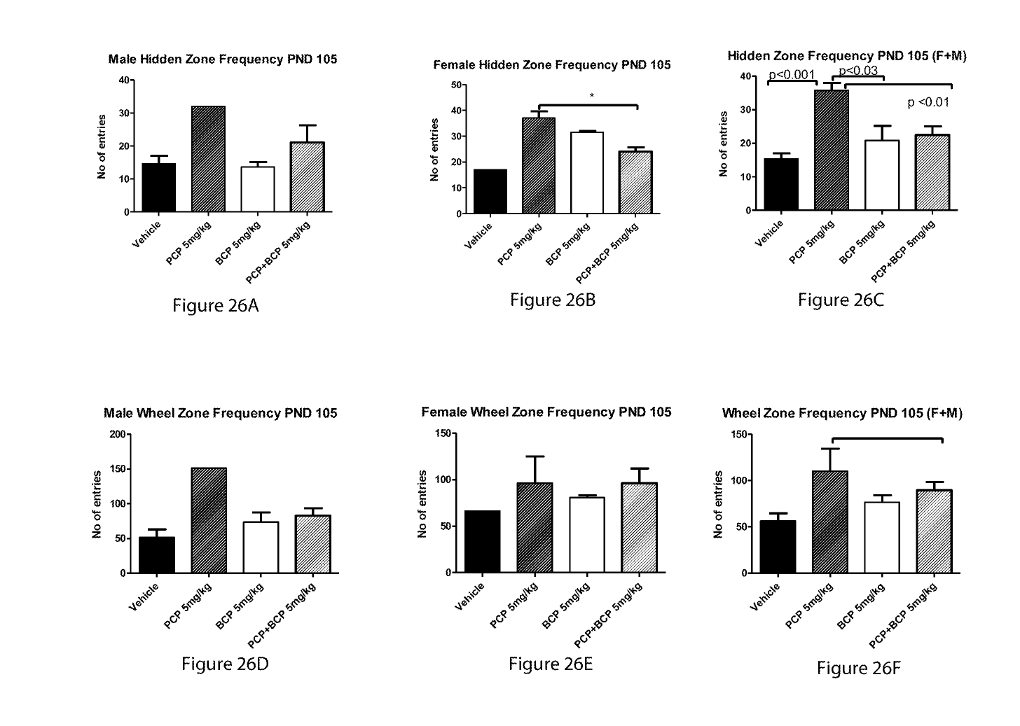

BCP treatment at adolescence reversed the effects of PCP on the frequency of entries to the hidden zone (FIGS. 26A, 26B, 26C). These results suggest that treatment with BCP reduced the level of stress and anxiety. BCP treatment appeared to reduce the effect of PCP on the frequency of entries to the Wheel Zone FIG. 26. This effect was prominent in males than in females (FIGS. 26D, 26E).

At PND 105, BCP treatment at adolescence reversed the effects of PCP on the time spend at the hidden zone (FIGS. 27A, 27B). These results suggest that treatment with BCP reduced the level of stress and anxiety. BCP treatment at adolescence showed a trend to reverse the effect of PCP on the time spend in the wheel (FIGS. 27C, 27D, 27E).

### Example 16

### III. Effects of AM630 (6-Iodopravadoline)

AM630 (6-Iodopravadoline, CAS 164178-33-0) is a molecule that acts as a potent and selective antagonist/inverse agonist for the cannabinoid receptor CB2, with a Ki of 32.1 nM at CB2 and 165× selectivity over CB1, at which it acted as a weak partial agonist. It is used in the study of CB2 mediated responses.

### Materials and Methods

### Murine Model of Schizophrenia:

Mice were injected with PCP (5 mg/kg in saline) at about postnatal days PND 4, 6, 8, 11, 13, 15, and 18 to provide a murine model of schizophrenia. A control group was injected with vehicle (0.6:1:18.4 DMSO:Cremophor EL:saline) alone. Each experiment was repeated twice. In each experiment, male mice were divided into 4 groups: Group 1: vehicle (n=4 pups, 1+3 pups, respectively);

Group 2: PCP (n=5 pups, 2+3 pups, respectively);

Group 3: PCP+BCP (n=6 pups, 2+4, respectively); and

Group 4:PCP+BCP+AM630 (n=3 pups, 1+2 respectively).

### Administration of BCP or BCP+AM630

The effect of co-administering AM630 with BCP was studied.

One hour after each injection with PCP, mice were injected with vehicle or BCP (final dose 10 mg/kg in 1:0.6:18 Cremophor EL:DMSO:saline) or BCP+AM630 (equal parts of 20 mg/kg BCP in DMSO and 20 mg/kg AM630 in DMSO, providing a final concentration of 10 mg/kg each of BCP and AM630, mixed together).

### Results

### Rearing and Exploration

At PND 17, locomotor activity, hyperactivity, exploratory and grooming behaviors were tested with the open-field test (FIGS. 29A and 29B). PCP significantly inhibited both ambulation and rearing behaviors. Treatment with BCP reversed the effects of PCP on rearing and exploration. AM630 reversed the effects of BCP on ambulation and rearing behaviors. The behaviors of mice that had been treated with AM630 were not significantly different from these of PCP-treated mice. AM630 is a selective CB2 receptor antagonist/inverse-agonist, therefore these results further support the CB2 receptor mediated effects of BCP.

### Example 17

II. Postnatal Induction of Schizophrenia (Days 3-15) Followed by Oral Treatment of Adolescent Mice with BCP.

### Methods

### Preparation of Diluted Oral Formulation of BCP for Administration by Gavage

BCP was diluted in canola oil. BCP (10 mg/kg diluted in canola oil) was administered to adolescent mice (PND 43-62) by gavage twice a week (on Sunday and Wednesday) for 3 weeks, a total of 6 gavages. Control group and PCP-induced group received by gavage the oil vehicle. After the final gavage, mice were tested in the open field test (PND 59), forced-swimming test (PND 83) and social interaction test (PND 88-89).

BCP was diluted in SEDDS vehicle. Oral 16% BCP composition in a SEDDS (self-emulsifying drug delivery system) vehicle. Preparation of the SEDDS vehicle:

### Vehicle

| Component | gram | % |
|---|---|---|
| MCT oil (Capric/caprylic triglycerides) NF | 38.4 | 38.40% |
| Labrafil M1944CS EP (Oleoyl polyoxyl-6 | 38.0 | 38.00% |
| glycerides) | | |
| Kollliphor EL NF (PEG 40 castor oil) | 7.25 | 7.25% |
| Polysorbate 60 (Tween-60) | 11.8 | 11.80% |
| Soy lecithin (Phosal 75 SA) | 2.95 | 2.95% |
| dl-alpha-Tocopherol USP | 1.6 | 1.60% |
| Total: | 100 | 100.0% |

The ingredients dl-alpha tocopherol and Phosal 75SA were stored in a refrigerator dl-Alpha tocopherol and Phosal 75SA were removed from refrigerator and allowed to reach room temperature while tightly closed.

Labrafil M1944CS and Polysorbate 60 were heated to 50-55° C. until each product becomes a clear and homogenous liquid.

The following ingredients were weighed into a 200 ml glass beaker weigh in the following order: dl-alpha Tocopherol (1.760 g), Phosal 75SA (3.245 g), Kolliphor EL (7.975 g), Polysorbate 60 (12.980 g), Labrafil M1944CS (41.800 g) and Capric/caprylic triglycerides (42.245 g)—Total: 110.00 g d˜0.962 g/ml

The beaker was covered and heated to 45-50° C. until all ingredients are completely melted.

The obtained liquid was mixed using a magnetic stirrer at medium/low speed until a homogenous liquid SEDDS vehicle was formed (10-20 minutes).

The SEDDS vehicle obtained as a hazy liquid was transferred to amber glass storage bottles and the head space was flushed with nitrogen. The bottles were tightly closed, sealed and stored in a refrigerator at +2-8° C.

### Preparation of the BCP Oral Composition in a SEDDS Vehicle

### Composition (16% BCP)

| Component | gram | % |
|---|---|---|
| MCT oil | 32.26 | 32.26% |
| Labrafil M1944CS | 31.92 | 31.92% |
| Kolliphor EL | 6.09 | 6.09% |
| Tween 60 | 9.91 | 9.91% |
| Phosal 75SA | 2.48 | 2.48% |
| BCP | 16.00 | 16.00% |
| dl-alpha-Tocopherol | 1.34 | 1.34% |
| Total: | 10.000 | 100.00% |

The SEDDS vehicle was stored in a refrigerator. The active agent BCP was stored in a freezer.

The vehicle and the active were removed from storage, allowed to reach room temperature while tightly closed, then warmed to 35-40° C. using a water bath. The vehicle was shaken to homogenize it.

SEDDS vehicle (84.0 g) was weighed into an Erlenmeyer flask with a stopper and BCP (16.0 g) was added to it. The flask was closed and mixed using a magnetic stirrer for 10-15 minutes at low speed until a homogenous mixture was formed.

The oral composition obtained was slightly cloudy/opalescent.

The above oral composition was filled into capsules or diluted with water, as per need.

Preparation of Diluted Oral SEDDS Vehicle with BCP for Administration by Gavage.

Sterile double-distilled water (DDW) was warmed for 10 min in a pre-warmed thermobath (35-38° C.). The SEDDS vehicle of the oral SEDDS composition was warmed up separately to 35-38° C. for 10 min. In order to prepare BCP (5 mg/ml) for a final dose of 10 mg/kg, BCP (5 mg) was added directly into the vehicle (1 ml) and vortexed for 1 min to obtain the oral composition. Then the warmed sterile DDW (4 ml) at 35-38° C. was added at a ratio of 1:5 oral composition: DDW dilution and the diluted composition was vortexed for 1 min. In order to prepare BCP for a final dose of 5 mg/kg, 500 μl of BCP at 5 mg/ml were diluted with 500 μl SEDDS vehicle (1:2 dilution). Then the warmed sterile DDW (4 ml) at 35-38° C. was added at a ratio of 1:5 oral composition:DDW dilution and the diluted composition was vortexed for 1 min.

BCP (5 mg/kg or 10 mg/kg in diluted self-emulsifying vehicle was administered to adolescent mice (10 μl/g) by gavage twice a week (on Sunday and Wednesday) for 3 weeks (PND 43-62), a total of 6 injections. Control group and PCP-induced group received by gavage the oral formulation solution without the drug. After the final BCP injection, mice were tested in the open field test, forced-swimming test, social interaction test and Novel

### Object Recognition Test.

Forced-Swimming Test

Training was conducted for 6 min a day before the test. Each mouse was placed into a transparent glass cylinder filled with fresh water at 25° C. On the test day, the total duration/frequency of immobility and climbing was counted every 2 minutes for 6 minutes. An increased immobility is an index of learning and habituation, therefore a positive behavioral adaptation with a stressful condition.

Social Interaction Test in a Novel Environment

Each mouse was placed in a novel cage together with a nonaggressive intruder mouse of the same species, same sex and a similar age. The interaction between the two mice was recorded for 10 minutes with EthoVision software (Noldus). Social interaction was defined by contact between the mice (tracking nose point). Reduced duration of contact behavior indicates on impairment in social interaction.

Cognitive Symptoms

Another test is the Novel Object Recognition (NOR) test used as an index for functional memory and learning processes, affected by different brain regions that are involved in the process of recognition. At age 10-12 weeks, the mice were exposed to two objects for a few minutes and returned to cage for one hour. One of the objects was replaced with a novel object and the time each mouse sniff, lick or touch the novel object was recorded using the EthoVision software (Noldus).

Open-Field Test (Crossing and Rearing)

Mice were assessed for hyperactivity behavior on postnatal day 64-66. Mice were placed in the center of a transparent glass cube cage 30×40 X31 cm divided into squares of 7.5×7.5 cm. The number of squares and rearing activity were counted for 8 min.

### Results

FIG. 29A shows that oral treatment with 10 mg/kg BCP reversed the effect of 5 mg/kg PCP on activity of female mice in the open field test. These results show that BCP acts orally.

FIG. 29B shows that oral treatment with 10 mg/kg BCP reversed the effect of 5 mg/kg PCP on the duration of immobility of male mice in the forced swim test. These results show that BCP is orally active and reverses depression-like behavior, supporting its use as a pharmaceutical drug for the treatment of mental diseases in which depression is one of the symptoms (like for example bi-polar/mania-depressive disorder, depression, anxiety, ADHD, Tourette syndrome, depression associated with neurodegenerative diseases, depression that leads to metabolic diseases).

FIG. 30A shows that oral treatment with 10 mg/kg BCP reversed the effect of 5 mg/kg PCP on social interaction of male mice in the social interaction test. These results show that orally administered BCP is effective in improving social interaction, supporting its use as a drug for the treatment mental diseases in which decrease of social interaction is one of the symptoms (like for example autism, Asperger syndrome, oppositional defiant disorder, personality disorders and avoidant personality disorder).

FIG. 30B shows that oral treatment with 10 mg/kg BCP did not affect body weight of male mice at postnatal day 83. These results further support BCP as contributing to body weight control.

FIG. 34 shows that oral treatment with 5 mg/kg BCP in SEDDS formulation reversed the effect of PCP in the open field test. These results further support that: (1) BCP is orally active; (2) BCP in SEDDS is orally active at the same dose as the dose of injection (see Example 15), these results were surprising as usually oral doses are 3-4 times higher than oral doses, therefore, the selected oral doses are similar to the doses of injections; (3) The effect of BCP in SEDDS was greater than the effect of vehicle only, showing a synergistic effect with SEDDS formulation. Thus SEDDS formulation with antioxidants not only stabilizes BCP but also enhance its effect.

### Example 18 II. Postnatal Induction of Schizophrenia (Days 3-15) Followed by Oral Treatment of Adolescent Mice with Risperidone

### Methods

### Preparation of Oral Formulation of Risperidone for Gavage Injection

Risperidone was from Sigma-Aldrich (Cat. R3030). Acetic acid 1% was prepared in sterile double distilled water (DDW) from acetic acid 5% solution (food grade). For example 5 ml of 5% acetic acid were mixed with 20 ml sterile DDW. Risperidone (1 mg) was dissolved in 20 ml of 1% acetic acid.

Risperidone (0.5 mg/kg in oral formulation) was administered by gavage to adolescent mice (PND 43-62) twice a week (on Sunday and Wednesday) for 3 weeks, a total of 6 injections. Control group and PCP-induced group received by gavage 1% acetic acid without the drug. After the final risperidone gavage, mice were tested in the open field test (PND 64).

### Results

FIG. 31A shows that oral treatment with 0.5 mg/kg risperidone reversed the effect of 5 mg/kg PCP on activity in the open field test of male mice at postnatal day 64. These results show that risperidone is orally active and reverses the schizophrenia-like effect of PCP in the same model that BCP was tested as mentioned above.

### Example 19

III. Postnatal Induction of Schizophrenia (Days 3-15) Followed by Treatment of Mice with HU-308.

### Methods

HU-308 (final dose 5 mg/kg in 1:0.6:18.4 Cremophor EL:ethanol:saline) was administered by injection 1 hour after PCP. Mice were tested at the age of 7-8 weeks in the PPI test.

### Results

FIG. 31B shows the effect of HU-308 in the PPI test. The prepulse inhibition test reflects the integrity of the sensor-motor gating system. Postnatal treatment with PCP reduced the percent inhibition, showing that the sensor-motor gating system is dysfunction. Postnatal treatment with HU-308 reversed the effect of PCP on the % PPI. These results show that HU-308, another CB2 selective agonist, reverses schizophrenia-like behavior.

### Example 20

### The DOI Model Supports LSD-Induced Psychosis and Supports the Activity of Selective CB2 Receptor Agonist as an Antipsychotic Against LSD-Induced Psychosis.

### Materials

The selective CB2 receptor agonist HU-308 was from Tocris. Cremophor EL and chemicals were purchased from Sigma-Aldrich, St. Louis, Mo., USA. BCP was provided by Prof Gertsch, University of Bern, Switzerland. The solution of HU-308 or BCP was prepared in Cremophor EL/ethanol/saline (1:0.6:18) or in Cremophor EL/DMSO/saline (1:0.6:18). The solution of DOI was prepared in saline.

Antipsychotic drugs were purchased from Sigma. Each drug was dissolved according to the manufacturer's instructions.

### Mice Models

HU-308, BCP and the antipsychotic drug were injected intraperitoneally at doses of 0.2 to 20 mg/kg, one to two hours before the injection of DOI. In order to test the effect of DOI on psychotic-like responses, DOI was intraperitoneally injected at a dose of 0.5 to 5 mg/kg.

For the control group, mice were injected intraperitoneally with an equivalent amount of the vehicle of Cremophor EL/ethanol/saline (1:0.6:18) or Cremophor EL/DMSO/saline (1:0.6:18) or oral formulation as listed above and saline according to the above procedure.

HU-308 or BCP or antipsychotic drug were injected intraperitoneally at doses of 0.01 to 100 mg/kg one to two hours before the injection of DOI. In order to test the effect of DOI on psychotic-like responses, DOI was intraperitoneally injected at 1 mg/kg. For the control group, mice pups were injected intraperitoneally with an equivalent amount of the vehicle EL/ethanol/saline (1:0.6:18) or cremophor EL/DMSO/saline (1:0.6:18) and saline, according to the above schedule.

### Results

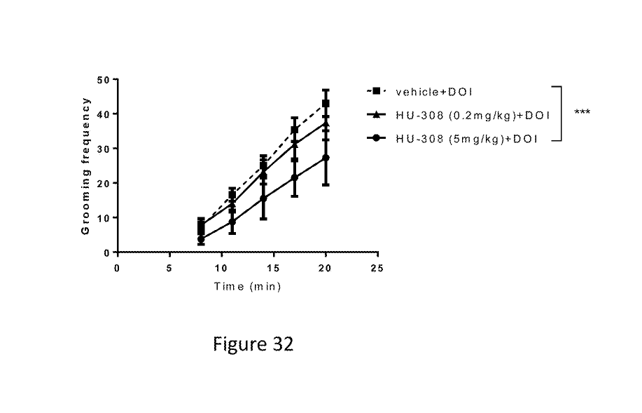

FIG. 32 shows the effect of HU-308 in the DOI test. DOI significantly increased the grooming response. HU-308 significantly reversed the effect of DOI on grooming response.

### Example 21

### CYP450 Enzymatic Metabolism: Interactions Between BCP and Antipsychotics

CYP450 dependent activities were determined using a known substrate. Enzyme activities of BCP or HU-308 were tested at various concentrations 1-3000 μM in the presence or absence of an antipsychotic drug at a fixed concentration equivalent to IC50. A known inhibitor, such as quinidine, tested at a single concentration 3.00 μM was included as positive control. Incubation mixture containing human liver microsomes (0.1 mg/mL), substrate (5 μM) and standard inhibitor or test compound were warmed at 37 C for 10 minutes. Reactions were initiated by addition of the NADPH (1 mM) and the mixture was incubated for 20 minutes at 37° C. water bath. The organic solvent in the reaction mixture was DMSO. After incubation, ice cold acetonitrile was added to terminate the reaction. Generation of metabolite from the substrate reactions was determined by LC-MS/MS and assessed based on peak area ratios of analyte/IS. Mean was used to calculate the extent of inhibition (expressed as % of control activity).

### Results

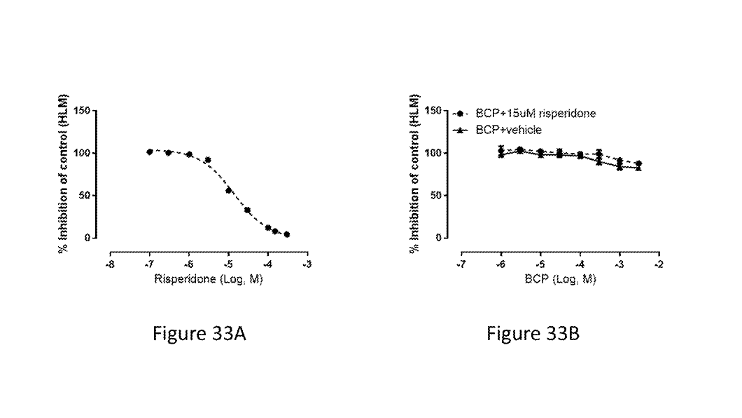

FIG. 33 shows the effect of BCP in the risperidone-induced CYP2D6 inhibition. Risperidone, an antipsychotic drug, inhibited CYP2D6 enzymatic activity. This inhibition produces toxicity in users. In contrast, BCP slightly inhibited CYP2D6. Combination of BCP with risperidone significantly reduced the risperidone-induced inhibition of CYP2D6. These results show that combination of BCP with antipsychotic agents has beneficial effect, reducing toxicity. These results further support that combination of CB2 selective agonist/s with antipsychotic agents further enhances their therapeutic effect.

### Example 22

Preparation of Sustained—Release BCP Tablets—50 mg/Tablet

Weigh 50 g BCP and mix with 20 g Vitamin E TPGS. The resulting mixture was mixed under controlled heat of not more than 50° C. and low speed of NMT 50 RPM with 300 g 101 grade microcrystallinecellulose (MCC). The mixing is continued until the oily mixture is completely absorbed. Next, the following polymers were added under mixing: Hydroxypropylmethylcellulose 15000 cp grade (K15M) 50 g, hydroxypropylmethylcellulose 4000 cp grade (K4M) 50 g and hydroxypropylmethylcellulose 100 cp grade (K15M) 100 g and mix the resulting mixture for 15 minutes. Add 1 g Aerosil 200 mix for 15 minutes, then add 1 g magnesium stearate and mix for additional 5 minutes. Compress the resulting composition into ER tablets containing 50 mg BCP/tablet.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

### REFERENCES

Anavi-Goffer, S., G. Baillie, A. J. Irving, J. Gertsch, I. R. Greig, R. G. Pertwee, and R. A. Ross. (2011) Modulation of L-alpha-lysophosphatidylinositol/GPR55 MAP kinase signalling by cannabinoids. J Biol Chem. 287: 91-104.
Ballmaier M, Bortolato M, Rizzetti C, Zoli M, Gessa G, Heinz A, Spano P. (2007) Cannabinoid receptor antagonists counteract sensorimotor gating deficits in the phencyclidine model of psychosis. Neuropsychopharmacology. 32: 2098-2107.
Darmani N A (2001) Cannabinoids of diverse structure inhibit two DOI-induced 5-HT(2A) receptor-mediated behaviors in mice. Pharmacol Biochem Behav 68: 311-317.
De Marchi N, De Petrocellis L, Orlando P, Daniele F, Fezza F, Di Marzo V (2003) Endocannabinoid signalling in the blood of patients with schizophrenia. Lipids Health Dis. 2:5.
Di Marzo V, Bifulco M, De Petrocellis L (2004) The endocannabinoid system and its therapeutic exploitation. Nat Rev Drug Discov. 3:771-784.
Fride E, Gobshtis N, Dahan H, Weller A, Giuffrida A, Ben-Shabat S (2009) The endocannabinoid system during development: emphasis on perinatal events and sustained effects. Vitam Horm. 81:139-58.
Gambi F, De Berardis D, Sepede G, Quartesan R, Calcagni E, Salerno R M, Conti C M, Ferro F M (2005) Cannabinoid receptors and their relationships with neuropsychiatric disorders. Int J Immunopathol Pharmacol. 18:9-25.
Gardner E L (2005) Endocannabinoid signaling system and brain reward: emphasis on dopamine. Pharmacol Biochem Behav. 81:263-284.
Gertsch J, et al. 2008. Beta-caryophyllene is a dietary cannabinoid. Proc Natl Acad Sci USA 105(26): 9099-9104.
Henstridge, C. M., N. A. Balenga, R. Schroder, J. K. Kargl, W. Platzer, L. Martini, S. Arthur, J. Penman, J. L. Whistler, E. Kostenis, M. Waldhoer, and A. J. Irving. (2010). GPR55 ligands promote receptor coupling to multiple signalling pathways. Br J Pharmacol. 160: 604-14.
Hashimoto K, Fujita Y, Shimizu E, Iyo M (2005) Phencyclidine-induced cognitive deficits in mice are improved by subsequent subchronic administration of clozapine, but not haloperidol. Eur J Pharmacol. 519:114-117.
Hanus L, Breuer A, Tchilibon S, Shiloah S, Goldenberg D, Horowitz M, et al. HU-308: a specific agonist for CB(2), a peripheral cannabinoid receptor. Proc Natl Acad Sci USA 1999; 96(25): 14228-33.
Josselyn S A and Vaccarino F J (1998) Preclinical behavioral approaches and study of antipsychotic drug action and schizophrenia, in in vivo neuromethods (Boulton A A, Baker G B and Bateson A N eds) pp 177-225, Humana Press, Totowa.
Leweke F M, Giuffrida A, Wurster U, Emrich H M, Piomelli D (1999) Elevated endogenous cannabinoids in schizophrenia. Neuroreport. 10:1665-1669.
Long L E, Malone D T, Taylor D A. (2006) Cannabidiol reverses MK-801-induced disruption of prepulse inhibition in mice. Neuropsychopharmacology. 4: 795-803.
Newell K A, Deng C, Huang X F. (2006) Increased cannabinoid receptor density in the posterior cingulate cortex in schizophrenia. Exp Brain Res. 172:556-60.
Ortega-Alvaro, A., A. Aracil-Fernandez, M. S. Garcia-Gutierrez, F. Navarrete, and J. Manzanares. (2011) Deletion of CB2 cannabinoid receptor induces schizophrenia-related behaviors in mice. Neuropsychopharmacology 36:1489.
Takahashi M, Kakita A, Futamura T, Watanabe Y, Mizuno M, Sakimura K, Castren E, Nabeshima T, Someya T, Nawa H (2006) Sustained brain-derived neurotrophic factor up-regulation and sensorimotor gating abnormality induced by postnatal exposure to phencyclidine: comparison with adult treatment. J Neurochem. 99:770-780.
Turgeon S M, Kim D, Pritchard M, Salgado S, Thaler A (2011) The effects of phenylcyclidine (PCP) on anxiety-like behavior in the elevated plus maze and the light-dark exploration test are age dependent, sexually dimorphic and task dependent.
Varty G B, Walters N, Cohen-Williams M, Carey G J (2001) Comparison of apomorphine, amphetamine and dizocilpine disruptions of prepulse inhibition in inbred and outbred mice strains. Eur J Pharmacol. 424:27-36.
Wang C Z & Johnson K M (2005) Differential effects of acute and subchronic administration on phencyclidine-induced neurodegeneration in the perinatal rat. J Neurosci Res. 81:284-292.
Wiley J L, Cristello A F, Balster R L (1995) Effects of site-selective NMDA receptor antagonists in an elevated plus-maze model of anxiety in mice. Eur J Pharmaco. 294: 101-107.

1. A method of treatment of a mental disorder in a patient in need thereof, wherein said method comprises:
administering a stable composition to a subject suffering from a mental disorder, wherein the stable composition comprises:
a therapeutically effective dose of at least one selective Cannabinoid Receptor Type 2 (CB2) receptor agonist, wherein the CB2 receptor selective agonist is selected from the group consisting of beta-caryophyllene (BCP), [(1R,2R,5R)-2-[2,6-dimethoxy-4-(2-methyloctan-2-yl)phenyl]-7,7-dimethyl-4-bicyclo[3.1.1]hept-3-enyl]methanol (“HU-308”) and combinations thereof;
at least one antioxidant, free-radical scavenger or a combination thereof selected from vitamin E, tocopherols, vitamin C, beta-carotene, butylated hydroxy toluene, butylated hydroxyanisole or other FDA-approved antioxidant listed in the FDA's Inactive Ingredients Database (IID), wherein the antioxidant and the selective Cannabinoid Receptor Type 2 agonist are in a ratio selected from 0.5:1 w/w to 1:0.5 w/w, from 0.5:1 w/w to 1:1 w/w, from 1:1 to 2:1, from 2:1 to 5:1 w/w, from 5:1 to 10:1 w/w, from 10:1 to 20:1 w/w, from 20:1 to 30:1 w/w, or from 30:1 to 40:1 w/w ratio of antioxidant/s to selective Cannabinoid Receptor Type 2 agonist;
a pharmaceutically effective carrier and
optionally at least one active agent selected from the group consisting of an antipsychotic agent, a GPR55 modulator, a terpene/terpenoid, an anti-inflammatory agent, an enzyme enhancer, an enzyme inhibitor, an antidepressant, an anxiolytic, a cognitive enhancer, an anti-diabetic agent, and combinations thereof,
wherein the at least one active agent co-administered in a single dosage form together with the at least one CB2 receptor selective agonist or co-administered sequentially in a dosage form separate from said CB2 receptor selective agonist in either order,
wherein the at least CB2 receptor agonist is in an amount sufficient to treat the patient suffering from the mental disorder.

2. (canceled)

3. (canceled)

4. (canceled)

5. (canceled)

6. (canceled)

7. The method of treatment of claim 1, wherein the mental disorder is selected from the group consisting of schizophrenia, bipolar disorder I and II, unipolar disorder, multiple personality disorder, psychotic disorders, depression, psychotic depression, depressive disorders, major depressive disorder, stereotypic movement disorder, autism spectrum disorders, obsessive-compulsive disorder (OCD), bacterial-induced tic disorder, pediatric autoimmune neuropsychiatric disorders associated with streptococcal infections (PANDAS), chorea (Sydenham's chorea (SC), chorea minor, chorea gravidarum, drug-induced chorea), drug-induced repetitive behaviors, akathisia, dyskinesias, Wernicke-Korsakoff syndrome, Tourette's syndrome, tic disorders, epilepsy, anxiety disorders, autistic spectrum disorder, enuresis, addiction, withdrawal symptoms associated with addiction, Asperger syndrome, oppositional defiant disorder, behavioral disturbance, agitation, psychosis/agitation associated with Alzheimer's disease, psychosis associated with Parkinson's disease, psychosis associated with drug of abuse, psychosis associated with psychedelic drug abuse, LSD-induced psychosis, steroid-induced schizophrenia, steroid-induced psychosis, Capgras syndrome; Fregoli syndrome; Cotard syndrome, personality disorders, borderline personality disorder, avoidant personality disorder, attention-deficit/hyperactive disorder (ADHD, ADD, HD), mania, dementia, anorexia, anorexia nervosa, anxiety, generalized anxiety disorder, social anxiety disorder, body dismographic disorder, obsessive compulsive disorder, paranoid disorder, nightmares, agitation, post-traumatic stress disorder (PTSD), severe mood dysregulation, depression or anxiety that leads to metabolic diseases, depression associated with any of the above clinical conditions and cognitive deficits associated with any of the above clinical conditions.

8. The method of treatment of claim 1, wherein said mental disorder is schizophrenia and wherein said schizophrenia includes any symptom and its onset is at any age.

9. The method of treatment of claim 1, wherein the mental disorder is schizophrenia of all types, the CB2 receptor selective agonist is BCP and the at least one active agent is selected from the group consisting of risperidone, paliperidone, paliperidone palmitate, aripiprazole, quetiapine, CBD, CBD derivatives, CBD analogs, CBG, CBG derivatives, CBG analogs, THCV, THCV derivatives, THCV analogs, brexpiprazole and combinations thereof.

10. The method of treatment of claim 1, wherein the at least one CB2 receptor selective agonist in substantially pure form is beta caryophyllene E-BCP and/or Z-BCP or HU-308 as sole active agent and the mental disorder is bi-polar disorder, having an onset at any age.

11. The method of treatment of claim 1, wherein the at least one CB2 selective receptor agonist is BCP or HU-308 as sole active agent and the mental disorder is selected from the group consisting of psychosis associated with psychedelic drug abuse and LSD-induced psychosis, having an onset at any age.

12. (canceled)

13. The method of treatment of claim 1, wherein the composition is administered to a patient in need thereof from once a month to once every two months, from once a month to once every three months, from once a month to once every four months, from once a month to once every five months, from once a month to once every six months, from once a month to once per week, twice per week, 3 times per week, 4 times per week, 5 times per week, 6 times per week, once per day, twice per day, 3 times per day, once a week to 3 times per day, once per week, twice per week, 3 times per week, 4 times per week, 5 times per week, 6 times per week, once per day, twice per day or 3 times per day.

14. (canceled)

15. (canceled)

16. The method of treatment of claim 1, wherein the average daily amount of CB2 receptor agonist selected from the group consisting of beta-caryophyllene (BCP), [(1R,2R,5R)-2-[2,6-dimethoxy-4-(2-methyloctan-2-yl)phenyl]-7,7-dimethyl-4-bicyclo[3.1.1]hept-3-enyl]methanol (HU-308) and combinations thereof administered is sufficient to treat the patient suffering from a mental disease by any daily mode of administration, wherein the average daily amount of CB2 receptor agonist is selected from 0.01-0.1 mg, 0.1-1 mg 1-10 mg, 10-25 mg, 25-100 mg, 100-1000 mg, according to the age and the effectiveness of the composition.

17. (canceled)

18. (canceled)

19. The method of treatment of claim 1, wherein the average daily amount of CB2 receptor agonist is sufficient to treat the patient suffering from a mental disease in a single administration of sustained-released delivery compositions selected from slow-release, slow-acting form of medication prepared as a capsule or depot injection administered mainly intramuscularly, once a week or once a month to up to once every six months, wherein the average daily amount of said CB2 receptor agonist administered is in a range selected 0.1-10 mg, 10-25 mg, 25-100 mg, 100-1000 mg or 100-3000 mg, according to the age and the effectiveness of the composition.

20. (canceled)

21. (canceled)

22. (canceled)

23. (canceled)

24. (canceled)

25. (canceled)

26. A stable composition, wherein the composition comprises:
at least one selective Cannabinoid Receptor Type 2 (CB2) receptor agonist, wherein the CB2 receptor selective agonist is selected from the group consisting of beta-caryophyllene (BCP), [(1R,2R,5R)-2-[2,6-dimethoxy-4-(2-methyloctan-2-yl)phenyl]-7,7-dimethyl-4-bicyclo[3.1.1]hept-3-enyl]methanol (HU-308) and combinations thereof;
at least one antioxidant, free-radical scavenger or a combination thereof selected from vitamin E, tocopherols, vitamin C, beta-carotene, butylated hydroxy toluene, butylated hydroxyanisole or other FDA-approved antioxidant listed in the FDA's Inactive Ingredients Database (IID), wherein the antioxidant and the selective Cannabinoid Receptor Type 2 agonist are in a ratio selected from 0.5:1 w/w to 1:0.5 w/w, from 0.5:1 w/w to 1:1 w/w, from 1:1 to 2:1, from 2:1 to 5:1 w/w, from 5:1 to 10:1 w/w, from 10:1 to 20:1 w/w, from 20:1 to 30:1 w/w, or from 30:1 to 40:1 w/w ratio of antioxidant/s to selective Cannabinoid Receptor Type 2 agonist;
optionally at least one active agent selected from the group consisting of an antipsychotic agent, a GPR55 modulator, a terpene/terpenoid, an anti-inflammatory agent, an enzyme enhancer, an enzyme inhibitor, an antidepressant, an anxiolytic, a cognitive enhancer, an anti-diabetic agent, and combinations thereof; and
a pharmaceutically effective carrier.

27. (canceled)

28. (canceled)

29. A stable composition, wherein the composition comprises:
at least one selective Cannabinoid Receptor Type 2 (CB2) receptor agonist wherein the at least one CB2 receptor agonist is selected from the group consisting of HU-433, HU-910, HU-914, CB 65, GP 1a, GP 2a, GW 405833, JWH 015, JWH 133, AM1241, L-759,656, L-759,633, MDA 19, SER 601, BML-190, N-alkylamide, rutamarin, diindolylmethane (DIM), cannabilactones, and combinations thereof;
optionally at least one active agent selected from the group consisting of an antipsychotic agent, a GPR55 modulator, a terpene/terpenoid, an anti-inflammatory agent, an enzyme enhancer, an enzyme inhibitor, an antidepressant, an anxiolytic, a cognitive enhancer, an anti-diabetic agent, and combinations thereof; and
a pharmaceutically effective carrier.

30. (canceled)

31. (canceled)

32. (canceled)

33. The composition of claim 26, wherein the at least one active agent is selected from the group consisting of haloperidol, chlorpromazine, fluphenazine, perphenazine, aripiprazole, clozapine, olanzapine, paliperidone, paliperidone palmitate, quetiapine, risperidone, ziprasidone, benperidol, bromperidol, droperidol, timiperone, fluspirilene, penfluridol, pimozide, acepromazine, cyamemazine, dixyrazine, levomepromazine, mesoridazine, perazine, pericyazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, amisulpride, amoxapine, dehydroaripiprazole, asenapine, cariprazine, blonanserin, iloperidone, lurasidone, melperone, nemonapride, perospirone, remoxipride, sertindole, sultopride, trimipramine, brexpiprazole, ITI-007, pimavanserin, RP5063 (RP5000), cannabidiol (CBD), cannabidivarin (CBDV), cannabiodiolic acid (CBDA), tetrahydrocannabivarin (THCV), OPC-14857, DM-1458, DM-1451, DM-1452, DM-1454, DCPP, cannabigerol (CBG), CBGA, CBGV, analogs thereof, derivatives thereof and combinations thereof.

34. The composition of claim 26, wherein the composition is stabilized and/or therapeutically enhanced by addition of an antioxidant, a free-radical scavenger or a combination thereof, selected from vitamin E, tocopherols, vitamin C, beta-carotene, butylated hydroxy toluene, butylated hydroxyanisole or other FDA-approved antioxidant listed in the FDA's Inactive Ingredients Database (IID) and wherein the antioxidant and the selective Cannabinoid Receptor Type 2 agonist are in a ratio selected from 0.5:1 w/w to 1:0.5 w/w, from 0.5:1 w/w to 1:1 w/w, from 1:1 to 2:1, from 2:1 to 5:1, w/w from 5:1 to 10:1 w/w, from about 10:1 to 20:1 w/w, from 20:1 to 30:1 w/w, or from 30:1 to 40:1 w/w ratio of antioxidant/s to selective Cannabinoid Receptor Type 2 agonist.

35. The composition of claim 26, wherein the composition is formulated for oral, parenteral, topical, intranasal, vaginal, inhalation, transdermal or rectal administration.

36. The composition of claim 35, wherein the composition is formulated as a tablet, sublingual tablet, caplet, depot, transdermal gel, cream, topical spray, nasal spray, transdermal patch, spray, suppository, chewable, capsule, dragee, powder, granules, suspension, solution, emulsion, syrup, transmucosal, lozenge, sachet, gastro-resistant oral dosage, gastroresistant softgel capsule, sprinkle or an ingestible solution.

37. The composition of claim 35, wherein the composition is formulated as an injectable solution and administered as intravenous injection, intra-arterial injection, intramuscular injection, intradermal injection, intraperitoneal injection, intrathecal injection, depot injection, subcutaneous injection or injectable suspension.

38. The composition of claim 26, the at least one active agent is selected from the group consisting of risperidone, paliperidone, paliperidone palmitate, aripiprazole, quetiapine, CBD, CBD derivatives, CBD analogs, CBG, CBG derivatives, CBG analogs, THCV, THCV derivatives, THCV analogs, brexpiprazole and combinations thereof.

39. (canceled)

40. (canceled)

41. (canceled)

42. (canceled)

43. (canceled)

44. The composition of claim 26, the composition comprising at least 98% w/w substantially pure isomer E-BCP, or at least 98% w/w substantially pure isomer Z-BCP, and wherein the composition is substantially free of BCP oxide and α-humulene.

45. (canceled)

46. The composition of claim 26, the composition comprising at least 85% w/w substantially pure isomer E-BCP or Z-BCP or E-BCP with Z-BCP and
optionally various amounts of alpha-humulene, copaene, eugenol, δ-cadinene, BCP oxide, and combinations thereof.

47. The composition of claim 26 the composition comprising at least 85% w/w substantially pure isomer E-BCP or at least 85% w/w substantially pure isomer Z-BCP, and 13% w/w alpha-humulene, 1% w/w copaene, 0.3% w/w eugenol, 0.3% w/w δ-cadinene and 0.3% w/w BCP oxide.

48. (canceled)
